SEQUENCE LISTING (26 sequences)

SEQ ID NO: 1 - protein, 5 aa, synthetic construct
  REGION 1..5: note = HCDR1, EGFR binding arm
TYGMH

SEQ ID NO: 2 - protein, 17 aa, synthetic construct
  REGION 1..17: note = HCDR2, EGFR binding arm
VIWDDGSYKYYGDSVKG

SEQ ID NO: 3 - protein, 16 aa, synthetic construct
  REGION 1..16: note = HCDR3, EGFR binding arm
DGITMVRGVMKDYFDY

SEQ ID NO: 4 - protein, 11 aa, synthetic construct
  REGION 1..11: note = LCDR1, EGFR binding arm
RASQDISSALV

SEQ ID NO: 5 - protein, 7 aa, synthetic construct
  REGION 1..7: note = LCDR2, EGFR binding arm
DASSLES

SEQ ID NO: 6 - protein, 9 aa, synthetic construct
  REGION 1..9: note = LCDR3, EGFR binding arm
QQFNSYPLT

SEQ ID NO: 7 - protein, 5 aa, synthetic construct
  REGION 1..5: note = HCDR1, c-Met binding arm
SYGIS

SEQ ID NO: 8 - protein, 17 aa, synthetic construct
  REGION 1..17: note = HCDR2, c-Met binding arm
WISAYNGYTNYAQKLQG

SEQ ID NO: 9 - protein, 10 aa, synthetic construct
  REGION 1..10: note = HCDR3, c-Met binding arm
DLRGTNYFDY

SEQ ID NO: 10 - protein, 11 aa, synthetic construct
  REGION 1..11: note = LCDR1, c-Met binding arm
RASQGISNWLA

SEQ ID NO: 11 - protein, 7 aa, synthetic construct
  REGION 1..7: note = LCDR2, c-Met binding arm
AASSLLS

SEQ ID NO: 12 - protein, 9 aa, synthetic construct
  REGION 1..9: note = LCDR3, c-Met binding arm
QQANSFPIT

SEQ ID NO: 13 - protein, 125 aa, synthetic construct [sequence omitted]

SEQ ID NO: 14 - protein, 107 aa, synthetic construct [sequence omitted]

SEQ ID NO: 15 - protein, 119 aa, synthetic construct
  REGION 1..119: note = VH, c-Met binding arm
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSS

SEQ ID NO: 16 - protein, 107 aa, synthetic construct
  REGION 1..107: note = VL, c-Met binding arm
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIK

SEQ ID NO: 17 - protein, 455 aa, synthetic construct
  REGION 1..455: note = Heavy chain 1
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 18 - protein, 214 aa, synthetic construct
  REGION 1..214: note = Light chain 1
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 449 aa, synthetic construct
  REGION 1..449: note = Heavy chain 2
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 20 - protein, 214 aa, synthetic construct
  REGION 1..214: note = Light chain 2
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 443 aa, Homo sapiens
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEP

SEQ ID NO: 22 - protein, 447 aa, Homo sapiens [sequence omitted]

SEQ ID NO: 23 - protein, 446 aa, Homo sapiens
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIF

SEQ ID NO: 24 - protein, 445 aa, Homo sapiens
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQI

SEQ ID NO: 25 - protein, 444 aa, Homo sapiens
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQ

SEQ ID NO: 26 - protein, 509 aa, Homo sapiens
  REGION 1..509: note = precursor human PH20
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

(19) United States
(12) Patent Application Publication
(10) Pub. No.: US 2025/0195649 A1
(43) Pub. Date: Jun. 19, 2025

(54) High Concentration Bispecific Antibody Formulations

(71) Applicant: Janssen Biotech, Inc., Horsham, PA (US)

(72) Inventors: Roland Knoblauch, Doylestown, PA (US); Satyen Torne, Chesterbrook, PA (US)

(21) Appl. No.: 19/072,332

(22) Filed: Mar. 6, 2025

Related U.S. Application Data
(63) Continuation of application No. 17/725,755, filed on Apr. 21, 2022.
(60) Provisional application No. 63/177,518, filed on Apr. 21, 2021.
(60) Provisional application No. 63/180,690, filed on Apr. 28, 2021.
(60) Provisional application No. 63/309,230, filed on Feb. 11, 2022.

(51) Int. Cl. A61K 39/395; A61K 9/00; A61K 39/00; A61K 47/12; A61K 47/18; A61K 47/20; A61K 47/26; A61K 47/42; A61P 35/00; C07K 16/28

(52) U.S. Cl. CPC A61K 39/39591; A61K 9/0019; A61K 47/12; A61K 47/183; A61K 47/20; A61K 47/26; A61K 47/42; A61P 35/00; C07K 16/2863; A61K 2039/505; A61K 2039/54; C07K 2317/31; C07K 2317/565

(57) ABSTRACT

Provided herein are stable aqueous pharmaceutical compositions comprising high concentration formulations of a bispecific epidermal growth factor receptor (EGFR)/hepatocyte growth factor receptor (c-Met) antibody and methods of preparing the same. Also provided herein are methods of treating cancer in a subject in need thereof by subcutaneously administering to the subject the stable aqueous pharmaceutical compositions as disclosed herein.

### CROSS-REFERENCE TO RELATED APPLICATIONS

This application is a continuation of U.S. patent application Ser. No. 17/725,755, filed 21 Apr. 2022, which claims priority to U.S. Provisional Application Ser. No. 63/177,518, filed 21 Apr. 2021, U.S. Provisional Application Ser. No. 63/180,690, filed 28 Apr. 2021, and U.S. Provisional Application Ser. No. 63/309,230, filed 11 Feb. 2022. The entire contents of the aforementioned applications are incorporated herein by reference in its entirety.

### REFERENCE TO SEQUENCE LISTING SUBMITTED ELECTRONICALLY

This application contains a sequence listing XML, which is submitted electronically as an XML formatted sequence listing with a file name “103693_004342_SL.xml”, creation date of Feb. 26, 2025 and having a size of 35,691 bytes. The sequence listing submitted electronically is part of the specification and is herein incorporated by reference in its entirety.

### FIELD OF THE INVENTION

Disclosed are compositions of and methods of using and formulating stable compositions comprising bispecific EGFR/c-Met antibodies for administration, including subcutaneous administration.

### BACKGROUND OF THE INVENTION

The role of both epidermal growth factor receptor (EGFR, ErbB1 or HER1) and hepatocyte growth factor receptor (c-Met) in cancer is well established, making these targets attractive for combination therapy. Both receptors signal through the same survival and anti-apoptotic pathways (ERK and AKT). Combination therapies targeting EGFR and c-Met or bispecific anti-EGFR/c-Met molecules have been tested in various clinical trials. While bispecific anti-EGFR/c-Met antibodies have shown promising results, there remains a need in the art for pharmaceutical compositions comprising such antibodies that are stable for long periods of time at refrigerated (2-8° C.) and ambient temperatures while being optimally formulated for their mode of administration.

### SUMMARY OF THE INVENTION

Disclosed herein are stable aqueous pharmaceutical compositions comprising specific formulations of a bispecific antibody.

In one aspect, provided herein are stable aqueous pharmaceutical compositions comprising a bispecific epidermal growth factor receptor (EGFR)/hepatocyte growth factor receptor (c-Met) antibody and a hyaluronidase, wherein the antibody comprises:
a first heavy chain (HC1) comprising a HC1 variable region 1 (VH1) comprising the amino acid sequence of SEQ ID NO:13;
a first light chain (LC1) comprising a light chain variable region 1 (VL1) comprising the amino acid sequence of SEQ ID NO:14;
a second heavy chain (HC2) comprising a HC2 variable region 2 (VH2) comprising the amino acid sequence of SEQ ID NO:15;
a second light chain (LC2) comprising a light chain variable region 2 (VL2) comprising the amino acid sequence of SEQ ID NO:16;
and wherein the composition comprises about 1,050 mg to about 2,240 mg of the bispecific EGFR/c-Met antibody and about 13,000 U to about 28,000 U of the hyaluronidase.

In some embodiments, the composition comprises about 1,050 mg of the bispecific EGFR/c-Met antibody.

In some embodiments, the composition comprises about 1,400 mg of the bispecific EGFR/c-Met antibody.

In some embodiments, the composition comprises about 1,575 mg of the bispecific EGFR/c-Met antibody.

In some embodiments, the composition comprises about 1,600 mg of the bispecific EGFR/c-Met antibody.

In some embodiments, the composition comprises about 2,100 mg of the bispecific EGFR/c-Met antibody.

In some embodiments, the composition comprises about 2,240 mg of the bispecific EGFR/c-Met antibody.

In one aspect, provided herein are stable aqueous pharmaceutical compositions comprising:
a) about 144 mg/mL to about 176 mg/mL of a bispecific epidermal growth factor receptor (EGFR)/hepatocyte growth factor receptor (c-Met) antibody, the bispecific antibody comprising:
   a first heavy chain (HC1) comprising a HC1 variable region 1 (VH1);
   a first light chain (LC1) comprising a light chain variable region 1 (VL1);
   a second heavy chain (HC2) comprising a HC2 variable region 2 (VH2); and
   a second light chain (LC2) comprising a light chain variable region 2 (VL2),
   wherein the VH1 comprises a heavy chain complementarity determining region 1 (HCDR1), a HCDR2 and a HCDR3 amino acid sequences of SEQ ID NOs: 1, 2, and 3, respectively; the VL1 comprises a light chain complementarity determining region 1 (LCDR1), a LCDR2 and a LCDR3 amino acid sequences of SEQ ID NOs: 4, 5 and 6, respectively, the VH2 comprises the HCDR1, the HCDR2 and the HCDR3 amino acid sequences of SEQ ID NOs: 7, 8 and 9, respectively; and the VL2 comprises the LCDR1, the LCDR2 and the LCDR3 amino acid sequences of SEQ ID NOs: 10, 11 and 12, respectively;
b) about 10 mM to about 50 mM of acetate and/or pharmaceutically acceptable acetate salt;
c) about 6.8% (w/v) to about 10.2% (w/v) of sucrose;
d) about 0.036% (w/v) to about 0.084% (w/v) of polysorbate 80 (PS80);
e) about to 0.8 mg/mL to about 1.2 mg/mL of methionine;
f) about 16 μg/mL to about 24 μg/mL of ethylenediaminetetraacetic acid (EDTA);
g) optionally, about 1,000 U/mL to about 3,000 U/mL of hyaluronidase; and
h) a pH from about 5.2 to about 6.2.

In some embodiments, the stable aqueous pharmaceutical composition comprises about 160 mg/mL of the bispecific EGFR-cMet antibody, about 30 mM acetate and/or pharmaceutically acceptable acetate salt, about 8.5% sucrose, and about 1 mg/mL L-methionine with polysorbate 80 to a final concentration of about 0.06% (w/v) and EDTA to a final concentration of about 20 μg/mL, wherein the stable aqueous pharmaceutical composition has pH about 5.7, and wherein the bispecific EGFR-cMet antibody comprises a heavy chain 1 (HC1) comprising the amino acid sequence of SEQ ID NO:17, HC2 comprising the amino acid sequence of SEQ ID NO:19, a light chain 1 (LC1) comprising the amino acid sequence of SEQ ID NO:18, and a LC2 comprising the amino acid sequence of SEQ ID NO:20.

In some embodiments, the stable aqueous pharmaceutical composition comprises about 160 mg/mL of the bispecific EGFR-cMet antibody, about 30 mM acetate and/or pharmaceutically acceptable acetate salt, about 8.5% sucrose, about 1 mg/mL L-methionine with polysorbate 80 to a final concentration of about 0.06% (w/v) and EDTA to a final concentration of about 20 μg/mL, and rHuPH20 to a final concentration of about 2,000 U/mL, wherein the stable aqueous pharmaceutical composition has pH about 5.7, and wherein the bispecific EGFR-cMet antibody comprises a heavy chain 1 (HC1) comprising the amino acid sequence of SEQ ID NO:17, HC2 comprising the amino acid sequence of SEQ ID NO:19, a light chain 1 (LC1) comprising the amino acid sequence of SEQ ID NO:18, and a LC2 comprising the amino acid sequence of SEQ ID NO:20.

Provided herein are also methods of treating cancer in a subject in need thereof. The methods comprise administering to the subject the stable aqueous pharmaceutical compositions as disclosed herein.

Also provided herein are also methods of reducing infusion-related reactions in a subject treated with amivantamab comprising subcutaneously administering to the subject the stable aqueous pharmaceutical formulation as disclosed herein.

Also provided herein are methods for preparing stable aqueous pharmaceutical compositions of a bispecific antibody targeting EGFR and cMet, the bispecific antibody targeting EGFR and cMet comprising a first heavy chain (HC1) comprising a HC1 variable region 1 (VH1); a first light chain (LC1) comprising a light chain variable region 1 (VL1); a second heavy chain (HC2) comprising a HC2 variable region 2 (VH2); and a second light chain (LC2) comprising a light chain variable region 2 (VL2), wherein the VH1 comprises a heavy chain complementarity determining region 1 (HCDR1), a HCDR2 and a HCDR3 comprising amino acid sequences of SEQ ID NOs: 1, 2, and 3, respectively; the VL1 comprises a light chain complementarity determining region 1 (LCDR1), a LCDR2 and a LCDR3 comprising amino acid sequences of SEQ ID NOs: 4, 5 and 6, respectively; the VH2 comprises HCDR1, HCDR2 and HCDR3 amino acid sequences of SEQ ID NOs: 7, 8 and 9, respectively; and the VL2 comprises LCDR1, LCDR2 and LCDR3 amino acid sequences of SEQ ID NOs: 10, 11 and 12, respectively. The methods comprise combining a composition comprising about 160 mg/mL of the bispecific antibody, about 30 mM acetate and/or pharmaceutically acceptable acetate salt, about 8.5% sucrose, and about 1 mg/mL L-methionine with polysorbate 80 to a final concentration of about 0.06% (w/v) and EDTA to a final concentration of about 20 μg/mL, optionally rHuPH20 to a final concentration of about 2,000 U/mL, wherein the stable aqueous pharmaceutical composition has about pH 5.7.

Also, provided herein are kits comprising the stable pharmaceutical aqueous formulations as disclosed herein and instructions for use thereof.

Further provided herein are articles of manufacture comprising a container holding the stable aqueous pharmaceutical formulations as disclosed herein.

### BRIEF DESCRIPTIONS OF THE DRAWINGS

FIG. 1 shows the serum concentration-time profile of amivantamab doses.

FIG. 2 shows the saturation of soluble free EGFR and MET after first SC amivantamab dose. LLOQ—lower limit of quantification; Pre—predose; “C”—indicates a treatment cycle (each cycle was 28 days); “D”—indicates day within the treatment cycle.

### DETAILED DESCRIPTION OF THE INVENTION

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed compositions and methods are not to be constrained by the correctness or incorrectness of any such suggested mechanism or mode of action or reason for improvement.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value. It is not intended that the scope of the invention be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed compositions and methods which are, for clarity, described herein in the context of separate embodiments, may also be provided in combination in a single embodiment. Conversely, various features of the disclosed compositions and methods that are, for brevity, described in the context of a single embodiment, may also be provided separately or in any subcombination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, “about” when used in reference to numerical ranges, cutoffs, or specific values is used to indicate that the recited values may vary by up to as much as 10% from the listed value. As many of the numerical values used herein are experimentally determined, it should be understood by those skilled in the art that such determinations can, and often times will, vary among different experiments. The values used herein should not be considered unduly limiting by virtue of this inherent variation. Thus, the term “about” is used to encompass variations of ±10% or less, variations of ±5% or less, variations of ±1% or less, variations of ±0.5% or less, or variations of ±0.1% or less from the specified value.

The term “comprising” is intended to include examples encompassed by the terms “consisting essentially of” and “consisting of”; similarly, the term “consisting essentially of” is intended to include examples encompassed by the term “consisting of.”

The term “antibody,” and like terms is meant in a broad sense and includes immunoglobulin molecules or fragments thereof including monoclonal antibodies (such as murine, human, human-adapted, humanized, and chimeric monoclonal antibodies), antibody fragments, bispecific or multispecific antibodies, dimeric, tetrameric or multimeric antibodies, and single chain antibodies.

Immunoglobulins can be assigned to five major classes, namely IgA, IgD, IgE, IgG, and IgM, depending on the heavy chain constant domain amino acid sequence. IgA and IgG are further sub-classified as the isotypes IgA1, IgA2, IgG1, IgG2, IgG3, and IgG4. Antibody light chains of any vertebrate species can be assigned to one of two clearly distinct types, namely kappa (κ) and lambda (λ), based on the amino acid sequences of their constant domains.

“Antibody fragment” refers to a portion of an immunoglobulin molecule that retains the antigen binding properties of the parental full-length antibody. Exemplary antibody fragments are heavy chain complementarity determining regions (HCDR) 1, 2, and 3, light chain complementarity determining regions (LCDR) 1, 2, and 3, a heavy chain variable region (VH), or a light chain variable region (VL). Antibody fragments include: a Fab fragment, a monovalent fragment consisting of the VL, VH, constant light (CL), and (constant heavy 1) CH1 domains; a F(ab)₂ fragment, a bivalent fragment comprising two Fab fragments linked by a disulfide bridge at the hinge region; a Fd fragment consisting of the VH and CHI domains; a Fv fragment consisting of the VL and VH domains of a single arm of an antibody; and a domain antibody (dAb) fragment (Ward et al., Nature 341:544-546, 1989), which consists of a VH domain. VH and VL domains can be engineered and linked together via a synthetic linker to form various types of single chain antibody designs where the VH/VL domains pair intramolecularly, or intermolecularly in those cases when the VH and VL domains are expressed by separate single chain antibody constructs, to form a monovalent antigen binding site, such as single chain Fv (scFv) or diabody; described for example in Int'l Pat. Pub. Nos. WO1998/44001, WO1988/01649, WO1994/13804, and WO1992/01047. These antibody fragments are obtained using techniques well known to those of skill in the art, and the fragments are screened for utility in the same manner as are full length antibodies.

An antibody variable region consists of a “framework” region interrupted by three “antigen binding sites.” The antigen binding sites are defined using various terms: (i) Complementarity Determining Regions (CDRs), three in the VH (HCDR1, HCDR2, HCDR3), and three in the VL (LCDR1, LCDR2, LCDR3) are based on sequence variability (Wu and Kabat J Exp Med 132:211-50, 1970; Kabat et al. Sequences of Proteins of Immunological Interest, 5th Ed. Public Health Service, National Institutes of Health, Bethesda, Md., 1991); and (ii) “Hypervariable regions” (“HVR” or “HV”), three in the VH (H1, H2, H3) and three in the VL (L1, L2, L3) refer to the regions of the antibody variable domains which are hypervariable in structure as defined by Chothia and Lesk (Chothia and Lesk Mol Biol 196:901-17, 1987). Other terms include “IMGT-CDRs” (Lefranc et al., Dev Comparat Immunol 27:55-77, 2003) and “Specificity Determining Residue Usage” (SDRU) (Almagro Mol Recognit 17:132-43, 2004). The International ImMunoGeneTics (IMGT) database (http://www_imgt_org) provides a standardized numbering and definition of antigen-binding sites. The correspondence between CDRs, HVs and IMGT delineations is described in Lefranc et al., Dev Comparat Immunol 27:55-77, 2003.

“Monoclonal antibody” refers to a preparation of antibody molecules of a single molecular composition. A monoclonal antibody composition displays a single binding specificity and affinity for a particular epitope, or in a case of a bispecific monoclonal antibody, a dual binding specificity to two distinct epitopes. Monoclonal antibody therefore refers to an antibody population with single amino acid composition in each heavy and each light chain, except for possible well-known alterations such as removal of C-terminal lysine from the antibody heavy chain. Monoclonal antibodies may have heterogeneous glycosylation within the antibody population. Monoclonal antibody may be monospecific or multispecific, or monovalent, bivalent or multivalent. A bispecific antibody is included in the term monoclonal antibody.

The term “biosimilar” (of an approved reference product/biological drug, i.e., reference listed drug) refers to a biological product that is highly similar to the reference product notwithstanding minor differences in clinically inactive components with no clinically meaningful differences between the biosimilar and the reference product in terms of safety, purity and potency, based upon data derived from (a) analytical studies that demonstrate that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is licensed and intended to be used and for which licensure is sought for the biosimilar. The biosimilar may be an interchangeable product that may be substituted for the reference product at the pharmacy without the intervention of the prescribing healthcare professional. To meet the additional standard of “interchangeability,” the biosimilar is to be expected to produce the same clinical result as the reference product in any given patient and, if the biosimilar is administered more than once to an individual, the risk in terms of safety or diminished efficacy of alternating or switching between the use of the biosimilar and the reference product is not greater than the risk of using the reference product without such alternation or switch. The biosimilar utilizes the same mechanisms of action for the proposed conditions of use to the extent the mechanisms are known for the reference product. The condition or conditions of use prescribed, recommended, or suggested in the labeling proposed for the biosimilar have been previously approved for the reference product. The route of administration, the dosage form, and/or the strength of the biosimilar are the same as those of the reference product and the biosimilar is manufactured, processed, packed or held in a facility that meets standards designed to assure that the biosimilar continues to be safe, pure and potent. The biosimilar may include minor modifications in the amino acid sequence when compared to the reference product, such as N- or C-terminal truncations that are not expected to change the biosimilar performance.

“Epitope” refers to a portion of an antigen to which an antibody specifically binds. Epitopes usually consist of chemically active (such as polar, non-polar, or hydrophobic) surface groupings of moieties such as amino acids or polysaccharide side chains and can have specific three-dimensional structural characteristics, as well as specific charge characteristics. An epitope can be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

“Hyaluronidase” refers to an enzyme that breaks down hyaluronic acid. This enzyme is often used to increase the dispersion and absorption of other co-administered drugs into tissue (e.g., subcutaneous injections, subcutaneous infusion such as hypodermoclysis). More specifically, human recombinant DNA-derived hyaluronidase enzyme PH20 (rHuPH20) is often used for increasing subcutaneous drug infusion particularly when large volumes are injected.

“Variant” refers to a polypeptide or a polynucleotide that differs from a reference polypeptide or a reference polynucleotide by one or more modifications for example, substitutions, insertions, or deletions.

“In combination with” means that two or more therapeutics can be administered to a subject together in a mixture, concurrently as single agents, or sequentially as single agents in any order.

“Treat,” “treatment,” and like terms refer to both therapeutic treatment and prophylactic or preventative measures, and includes reducing the severity and/or frequency of symptoms, eliminating symptoms and/or the underlying cause of the symptoms, reducing the frequency or likelihood of symptoms and/or their underlying cause, improving or remediating damage caused, directly or indirectly, by the malignancy. Treatment also includes prolonging survival as compared to the expected survival of a subject not receiving treatment. Subjects to be treated include those that have the condition or disorder as well as those prone to have the condition or disorder or those in which the condition or disorder is to be prevented.

“Therapeutically effective amount” refers to an amount of the disclosed combination therapy effective, at dosages and for periods of time necessary, to achieve a desired treatment. A therapeutically effective amount may vary according to factors such as the disease state, age, sex, and weight of the subject, and the ability of the combination therapy to elicit a desired response in the subject. Exemplary indicators of a therapeutically effect amount include, for example, improved well-being of the patient, reduction of a tumor burden, arrested or slowed growth of a tumor, and/or absence of metastasis of cancer cells to other locations in the body.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, solid malignancies such as breast cancer (BC), prostate cancer, ovarian cancer (OC), cervical cancer, skin cancer, pancreatic cancer, gastroesophageal cancer (GEC), colorectal cancer (CRC), renal cell cancer (RCC), liver cancer, hepatocellular cancer (HCC), brain cancer, squamous cell carcinoma of the head and neck (SCCHN)), lymphoma, leukemia, lung cancer (e.g. non-small cell lung cancer (NSCL) or small cell lung cancer (SCLC)), medullary thyroid cancer (MTC), and mesothelioma. The solid malignancy may be metastatic or unresectable. The solid malignancy may be histologically or cytologically confirmed.

Reference Material (RM) as used herein refers to a vial that contains about 0.2 mL aliquot of amivantamab, or biosimilar thereof, and is used in good manufacturing practices (GMP) for clinical drug substance. The RM is stored at at least −60° C. and is thawed and used as a control in amivantamab analytical assays.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” can be used interchangeably herein.

“Infusion-related reactions” or “IRRs” may occur in some patients to whom the bi-specific EGFR/c-Met antibody, such as amivantamab, is administered. Signs and symptoms of IRRs may include dyspnea, flushing, fever, chills, nausea, chest discomfort, hypotension, and/or vomiting. Systemic IRRs, including severe reactions, upon the introduction of a new protein therapeutic infusion may also occur.

### DESCRIPTION

Disclosed herein are stable, aqueous pharmaceutical compositions comprising a bispecific EGFR/c-Met antibody. The bispecific anti-EGFR/c-Met antibody may be provided in suitable pharmaceutical compositions comprising the bispecific anti-EGFR/c-Met antibody and a pharmaceutically acceptable carrier. The stable, aqueous pharmaceutical compositions may include one or more diluents, adjuvants, excipients, or vehicles with which the bispecific anti-EGFR/c-Met antibody is administered subcutaneously. Exemplary excipients include one or more of buffering agents, stabilizers, chelating agents, surfactants, and enzymes. According to some embodiments, the stable, aqueous pharmaceutical compositions comprising a bispecific EGFR/c-Met antibody further comprise a buffering agent, a stabilizer, a chelating agent, a surfactant, and, optionally, a hyaluronidase. The stable, aqueous pharmaceutical compositions comprising a bispecific EGFR/c-Met antibody provided herein are also referred to as drug product or DP.

In some aspects, the bispecific EGFR/c-Met antibody comprises a first heavy chain (HC1) comprising a HC1 variable region 1 (VH1); a first light chain (LC1) comprising a light chain variable region 1 (VL1); a second heavy chain (HC2) comprising a HC2 variable region 2 (VH2); and a second light chain (LC2) comprising a light chain variable region 2 (VL2), wherein the VH1 comprises a heavy chain complementarity determining region 1 (HCDR1), a HCDR2 and a HCDR3 amino acid sequences of SEQ ID NOs: 1, 2, and 3, respectively; the VL1 comprises a light chain complementarity determining region 1 (LCDR1), a LCDR2 and a LCDR3 amino acid sequences of SEQ ID NOs: 4, 5 and 6, respectively; the VH2 comprises the HCDR1, the HCDR2 and the HCDR3 amino acid sequences of SEQ ID NOs: 7, 8 and 9, respectively; and the VL2 comprises the LCDR1, the LCDR2 and the LCDR3 amino acid sequences of SEQ ID NOs: 10, 11 and 12, respectively (see table 29).

In some embodiments, the first heavy chain (HC1) of the bispecific EGFR-cMet antibody comprises a HC1 constant domain 3 (HC1 CH3) and a HC1 variable region 1 (VH1). In some embodiments, the second heavy chain (HC2) of the bispecific EGFR-cMet antibody comprises a HC2 constant domain 3 (HC2 CH3) and a HC2 variable region 2 (VH2). In some embodiments, the first heavy chain (HC1) of the bispecific EGFR-cMet antibody comprises a HC1 constant domain 3 (HC1 CH3) and a HC1 variable region 1 (VH1 and the second heavy chain (HC2) of the bispecific EGFR-cMet antibody comprises a HC2 constant domain 3 (HC2 CH3) and a HC2 variable region 2 (VH2). In some embodiments, the first heavy chain (HC1) of the bispecific EGFR-cMet antibody comprises a HC1 constant domain 2 and constant domain 3 (HC1 CH2-CH3) and a HC1 variable region 1 (VH1). In some embodiments, the second heavy chain (HC2) of the bispecific EGFR-cMet antibody comprises a HC2 constant domain 2 and constant domain 3 (HC2 CH2-CH3) and a HC2 variable region 2 (VH2). In some embodiments, the first heavy chain (HC1) of the bispecific EGFR-cMet antibody comprises a HC1 constant domain 2 and constant domain 3 (HC1 CH2-CH3) and a HC1 variable region 1 (VH1) and the second heavy chain (HC2) of the bispecific EGFR-cMet antibody comprises a HC2 constant domain 2 and constant domain 3 (HC2 CH2-CH3) and a HC2 variable region 2 (VH2).

In some embodiments, the bispecific antibody comprises asymmetric stabilizing mutations in the HC1 CH2-CH3 region, in the HC2 CH2-CH3 region, or both. “Asymmetric stabilizing mutations” refers to mutations in a first CH2-CH3 region and in a second CH2-CH3 region which are at different positions in the first and in the second CH2-CH3 region and favor (e.g., stabilize) heterodimer formation between the first CH2-CH3 region and the second CH2-CH3 region over homodimer formation between the first CH2-CH3 region or the second CH2-CH3 region. Exemplary asymmetric stabilizing mutations in the HC1 CH2-CH3 region and the HC2 CH2-CH3 region, or in the HC2 CH2-CH3 region and the HC1 CH2-CH3 region, are (wherein residue numbering is according to the EU Index):
F405L and K409R, respectively;
wild-type and F405L/R409K, respectively;
T366W and T366S/L368A/Y407V, respectively;
T366Y/F405A and T394W/Y407T, respectively;
T366W/F405W and T394S/Y407A, respectively;
F405W/Y407A and T366W/T394S, respectively;
L351Y/F405A/Y407V and T394W, respectively;
T366I/K392M/T394W and F405A/Y407V, respectively;
T366L/K392M/T394W and F405A/Y407V, respectively;
L351Y/Y407A and T366A/K409F, respectively;
L351Y/Y407A and T366V/K409F, respectively;
Y407A and T366A/K409F, respectively;
D399K/E356K and K409D/K392D, respectively; or
D399K/E356K/E357K and K409D/K392D/K370, respectively.

In some embodiments, the bispecific EGFR-cMet antibody comprises an HC1 variable region comprising the amino acid sequence of SEQ ID NO:13 and a LC1 variable region comprising the amino acid sequence of SEQ ID NO:14 (see table 29). In some embodiments, the bispecific antibody comprises asymmetric stabilizing mutations in the HC1 CH2-CH3 region, in the HC2 CH2-CH3 region, or both. In some embodiments, the bispecific antibody comprises K409R in the c-Met binding arm and F405L in the EGFR binding arm.

In some embodiments, the bispecific EGFR-cMet antibody comprises a HC2 variable region comprising the amino acid sequence of SEQ ID NO:15 and a LC2 variable region comprising the amino acid sequence of SEQ ID NO:16 (see table 29).

In some embodiments, the heavy chain 1 (HC1) comprises the amino acid sequence of SEQ ID NO:17 and the HC2 comprises the amino acid sequence of SEQ ID NO:19 (see table 29).

In some embodiments, the light chain 1 (LC1) comprises the amino acid sequence of SEQ ID NO:18 and the LC2 comprises the amino acid sequence of SEQ ID NO:20 (see table 29).

In some embodiments, bispecific EGFR-cMet antibody is amivantamab or a biosimilar thereof.

### Amivantamab

Amivantamab (JNJ-61186372) is a low fucose, fully human IgG1-based bispecific antibody directed against EGFR and cMET tyrosine kinase receptors that is approved by the FDA for patients with EGFR Exon 20ins mutations after treatment with chemotherapy.

Amivantamab shows activity against tumors with primary activating EGFR mutations (Exon 19 deletion [Exon 19del], Exon 21 leucine 858 to arginine substitution ([L858R], and Exon 20ins mutations), EGFR resistance mutations (tyrosine 790 to methionine [T790M] or cysteine 797 to serine [C797S] mutations), overexpressed wild type EGFR, and activation of the cMet pathway.

Lazertinib is an oral, highly potent, mutant-selective, and irreversible third-generation EGFR TKI that targets both the Exon 19del and the Exon 21 L858R EGFR activating mutations, as well as the T790M resistance mutation. Lazertinib has demonstrated efficacy in participants with EGFR-mutated NSCLC, with activity observed in both systemic and central nervous system lesions, demonstrating its ability to cross the blood-brain barrier. Combining lazertinib, which targets the intracellular EGFR tyrosine kinase site, with amivantamab, which targets the extracellular EGFR ligand-binding domains, has the potential to more potently inhibit the EGFR signaling pathway, attenuate frequent EGFR dependent and independent resistance mechanisms to EGFR TKIs, and induce deeper responses than either agent alone.

According to some aspects, the stable aqueous pharmaceutical composition comprises the bispecific EGFR-cMet antibody at a concentration of about: 100 mg/mL, 110 mg/mL, 120 mg/mL, 121 mg/mL, 122 mg/mL, 123 mg/mL, 124 mg/mL, 125 mg/mL, 126 mg/mL, 127 mg/mL, 128 mg/mL, 129 mg/mL, 130 mg/mL, 131 mg/mL, 132 mg/mL, 133 mg/mL, 134 mg/mL, 135 mg/mL, 136 mg/mL, 137 mg/mL, 138 mg/mL, 139 mg/mL, 140 mg/mL, 141 mg/mL, 142 mg/mL, 143 mg/mL, 144 mg/mL, 145 mg/mL, 146 mg/mL, 147 mg/mL, 148 mg/mL, 150 mg/mL, 151 mg/mL, 152 mg/mL, 153 mg/mL, 154 mg/mL, 155 mg/mL, 156 mg/mL, 157 mg/mL, 158 mg/mL, 159 mg/mL, 160 mg/mL, 161 mg/mL, 162 mg/mL, 163 mg/mL, 164 mg/mL, 165 mg/mL, 166 mg/mL, 167 mg/mL, 168 mg/mL, 169 mg/mL, 170 mg/mL, 171 mg/mL, 172 mg/mL, 173 mg/mL, 174 mg/mL, 175 mg/mL, 176 mg/mL, 177 mg/mL, 178 mg/mL, 179 mg/mL, 180 mg/mL, 181 mg/mL, 182 mg/mL, 183 mg/mL, 184 mg/mL, 185 mg/mL, 186 mg/mL, 187 mg/mL, 188 mg/mL, 190 mg/mL, 191 mg/mL, 192 mg/mL, 193 mg/mL, 194 mg/mL, 195 mg/mL, 196 mg/mL, 197 mg/mL, 198 mg/mL, 199 mg/mL or 200 mg/mL. In some embodiments, the bispecific EGFR-cMet antibody has a concentration of about 160 mg/mL.

In some embodiments, the bispecific anti-EGFR/c-Met antibody is administered at a dose of between about 140 mg to about 1750 mg. In some embodiments, the bispecific anti-EGFR/c-Met antibody is administered at a dose of between about 140 mg to about 2100 mg. In some embodiments, the bispecific anti-EGFR/c-Met antibody is administered at a dose of between about 1050 mg to about 2240 mg.

In some embodiments, the bispecific anti-EGFR/c-Met antibody is administered at a dose of about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1010 mg, about 1020 mg, about 1030 mg, about 1040 mg, about 1050 mg, about 1060 mg, about 1070 mg, about 1080 mg, about 1090 mg, about 1100 mg, about 1110 mg, about 1120 mg, about 1130 mg, about 1140 mg, about 1150 mg, about 1160 mg, about 1170 mg, about 1180 mg, about 1190 mg, about 1200 mg, about 1210 mg, about 1220 mg, about 1230 mg, about 1240 mg, about 1250 mg, about 1260 mg, about 1270 mg, about 1280 mg, about 1290 mg, about 1300 mg, about 1310 mg, about 1320 mg, about 1330 mg, about 1340 mg, about 1350 mg, about 1360 mg, about 1370 mg, about 1380 mg, about 1390 mg, about 1400 mg, about 1410 mg, about 1420 mg, about 1430 mg, about 1440 mg, about 1450 mg, about 1460 mg, about 1470 mg, about 1480 mg, about 1490 mg, about 1500 mg, about 1510 mg, about 1520 mg, about 1530 mg, about 1540 mg, about 1550 mg, about 1560 mg, about 1570 mg, about 1575 mg, about 1580 mg, about 1590 mg, about 1600 mg, about 1610 mg, 1620 mg, about 1630 mg, about 1640 mg, about 1650 mg, about 1660 mg, about 1670 mg, about 1680 mg, about 1690 mg, about 1700 mg, about 1710 mg, about 1720 mg, about 1730 mg, about 1740 mg, about 1750 mg, about 1760 mg, about 1770 mg, about 1780 mg, about 1790 mg, about 1800 mg, about 1810 mg, about 1820 mg, about 1830 mg, about 1840 mg, about 1850 mg, about 1860 mg, about 1870 mg, about 1880 mg, 1890 mg, about 1900 mg, about 1910 mg, about 1920 mg, about 1930 mg, about 1940 mg, about 1950 mg, about 1960 mg, about 1970 mg, about 1980 mg, about 1990 mg, about 2000 mg, about 2010 mg, about 2020 mg, about 2030 mg, about 2040 mg, about 2050 mg, about 2060 mg, about 2070 mg, about 2080 mg, about 2090 mg, about 2100 mg, about 2110 mg, about 2120 mg, about 2150 mg, about 2200 mg, about 2210 mg, about 2220 mg, about 2230 mg, about 2240 mg, about 2250 mg, about 2260 mg, about 2270 mg, about 2280 mg, about 2290 mg, or about 2300 mg.

In some embodiments, the bispecific anti-EGFR/c-Met antibody is administered at a dose of about 350 mg, about 700 mg, about 1050 mg, about 1400 mg, about 1575 mg, or about 2100 mg. In some embodiments, the bispecific anti-EGFR/c-Met antibody is administered at a dose of about 350 mg. In some embodiments, the bispecific anti-EGFR/c-Met antibody is administered at a dose of about 700 mg. In some embodiments, the bispecific anti-EGFR/c-Met antibody is administered at a dose of about 750 mg. In some embodiments, the bispecific anti-EGFR/c-Met antibody is administered at a dose of about 800 mg. In some embodiments, the bispecific anti-EGFR/c-Met antibody is administered at a dose of about 850 mg. In some embodiments, the bispecific anti-EGFR/c-Met antibody is administered at a dose of about 900 mg. In some embodiments, the bispecific anti-EGFR/c-Met antibody is administered at a dose of about 950 mg. In some embodiments, the bispecific anti-EGFR/c-Met antibody is administered at a dose of about 1000 mg. In some embodiments, the bispecific anti-EGFR/c-Met antibody is administered at a dose of about 1050 mg. In some embodiments, the bispecific anti-EGFR/c-Met antibody is administered at a dose of about 1100 mg. In some embodiments, the bispecific anti-EGFR/c-Met antibody is administered at a dose of about 1150 mg. In some embodiments, the bispecific anti-EGFR/c-Met antibody is administered at a dose of about 1200 mg. In some embodiments, the bispecific anti-EGFR/c-Met antibody is administered at a dose of about 1250 mg. In some embodiments, the bispecific anti-EGFR/c-Met antibody is administered at a dose of about 1300 mg. In some embodiments, the bispecific anti-EGFR/c-Met antibody is administered at a dose of about 1350 mg. In some embodiments, the bispecific anti-EGFR/c-Met antibody is administered at a dose of about 1400 mg. In some embodiments, the bispecific anti-EGFR/c-Met antibody is administered at a dose of about 1575 mg. In some embodiments, the bispecific anti-EGFR/c-Met antibody is administered at a dose of about 1600 mg. In some embodiments, the bispecific anti-EGFR/c-Met antibody is administered at a dose of about 2100 mg. In some embodiments, the bispecific anti-EGFR/c-Met antibody is administered at a dose of about 2240 mg.

In some embodiments, the bispecific anti-EGFR/c-Met antibody is administered once a week. In some embodiments, the bispecific anti-EGFR/c-Met antibody is administered about 1050 mg once a week. In some embodiments, the bispecific anti-EGFR/c-Met antibody is administered about 1400 mg once a week. In some embodiments, the bispecific anti-EGFR/c-Met antibody is administered about 1575 mg once a week. In some embodiments, the bispecific anti-EGFR/c-Met antibody is administered about 1600 mg once a week. In some embodiments, the bispecific anti-EGFR/c-Met antibody is administered about 2100 mg once a week. In some embodiments, the bispecific anti-EGFR/c-Met antibody is administered about 2240 mg once a week.

In some embodiments, the bispecific anti-EGFR/c-Met antibody is administered once in two weeks. In some embodiments, the bispecific anti-EGFR/c-Met antibody is administered about 1050 mg once in two weeks. In some embodiments, the bispecific anti-EGFR/c-Met antibody is administered about 1400 mg once in two weeks. In some embodiments, the bispecific anti-EGFR/c-Met antibody is administered about 1575 mg once in two weeks. In some embodiments, the bispecific anti-EGFR/c-Met antibody is administered about 1600 mg once in two weeks. In some embodiments, the bispecific anti-EGFR/c-Met antibody is administered about 2100 mg once in two weeks. In some embodiments, the bispecific anti-EGFR/c-Met antibody is administered about 2240 mg once in two weeks.

In some embodiments, the bispecific anti-EGFR/c-Met antibody is administered twice a week. In some embodiments, the bispecific anti-EGFR/c-Met antibody is administered once a week. In some embodiments, the bispecific anti-EGFR/c-Met antibody is administered once in two weeks. In some embodiments, the bispecific anti-EGFR/c-Met antibody is administered once in three weeks. In some embodiments, the bispecific anti-EGFR/c-Met antibody is administered once in four weeks.

For combination therapies, the one or more anti-cancer agents may be administered using recommended dosages of the anti-cancer agent.

The buffering agent is suitable to adjust the pH to about 5.0 to 6.2, such as pH 5.1 to 5.7. An exemplary buffer is a histidine buffer or acetate buffer. According to some aspects, the stable aqueous pharmaceutical composition comprises histidine and/or pharmaceutically acceptable histidine salt at a concentration of about: 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 46 mM, 47 mM, 48 mM, 49 mM or 60 mM. In some embodiments, the histidine and/or pharmaceutically acceptable histidine salts has a concentration of about 10 mM. In some embodiments, the histidine and/or pharmaceutically acceptable histidine salts has a concentration of about 50 mM. In a further embodiment, the histidine and/or pharmaceutically acceptable histidine salt comprises L-histidine and L-histidine hydrochloride monohydrate. According to some aspects, the stable aqueous pharmaceutical composition comprises acetate and/or pharmaceutically acceptable acetate salt at a concentration of about: 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, or 50 mM. In some embodiments, the acetate and/or pharmaceutically acceptable acetate salts has a concentration of about 30 mM. In a further embodiment, the acetate and/or pharmaceutically acceptable acetate salt comprises glacial acetic acid and/or sodium acetate trihydrate.

The stabilizer may comprise sucrose and optionally methionine. According to some aspects, the stable aqueous pharmaceutical composition comprises sucrose at a concentration (percentage of weight to volume (% w/v)) of about: 6.0%, 6.1%, 6.2%, 6.3%, 6.4%, 6.5%, 6.6%, 6.7% 6.8%, 6.9%, 7.0%, 7.1%, 7.2%, 7.3%, 7.4%, 7.5%, 7.6%, 7.7% 7.8%, 7.9%, 8.0%, 8.1%, 8.2%, 8.3%, 8.4%, 8.5%, 8.6%, 8.7% 8.8%, 9.9%, 10.0%, 10.1%, 10.2%, 10.3%, 10.4%, 10.5%, 10.6%, 10.7% 10.8%, 10.9%, or 11.0%. In some embodiments, the stable aqueous pharmaceutical composition comprises about 8.5% (w/v) sucrose. According to some aspects, the stable aqueous pharmaceutical composition comprises methionine (e.g. L-methionine) at a concentration of about: 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL, or 2.0 mg/mL. In one embodiment, the methionine comprises L-methionine and has a concentration of about 1.0 mg/mL.

A preferred surfactant is polysorbate 80 (PS80). According to some aspects, the stable aqueous pharmaceutical composition comprises polysorbate 80 (PS80) at a concentration (% w/v) of about: 0.005%, 0.01%, 0.015%, 0.020%, 0.025%, 0.030%, 0.035%, 0.036%, 0.037%, 0.038%, 0.039%, 0.040%, 0.041%, 0.042%, 0.043% 0.044%, 0.045%, 0.046%, 0.047%, 0.048%, 0.049%, 0.050%, 0.051%, 0.052%, 0.053%, 0.054%, 0.055%, 0.056%, 0.057%, 0.058% 0.059%, 0.060%, 0.061%, 0.062%, 0.063%, 0.064%, 0.065%, 0.066%, 0.067%, 0.068% 0.069%, 0.070%, 0.071%, 0.072%, 0.073%, 0.074%, 0.075%, 0.080%, 0.081%, 0.082%, 0.083%, 0.084%, 0.085%, 0.086%, 0.087%, 0.088% 0.089%, 0.090%, 0.091%, 0.092%, 0.093%, 0.094%, or 0.095%. In some embodiments, the stable aqueous pharmaceutical composition comprises about 0.06% (w/v) PS80.

A preferred chelating agent is ethylenediaminetetraacetic acid (EDTA). According to some aspects, the stable aqueous pharmaceutical composition comprises EDTA at a concentration of about: 10 μg/mL, 11 μg/mL, 12 μg/mL, 13 μg/mL, 14 μg/mL, 15 μg/mL, 16 μg/mL, 17 μg/mL, 18 μg/mL, 19 μg/mL, 20 μg/mL, 21 μg/mL, 22 μg/mL, 23 μg/mL, 24 μg/mL, 25 μg/mL, 26 μg/mL, 27 μg/mL, 28 μg/mL, 29 μg/mL, or 30 μg/mL. In one embodiment, the EDTA has a concentration of about 20 μg/mL.

In some embodiments, the stable aqueous pharmaceutical composition comprising a bispecific EGFR/c-Met antibody comprises a hyaluronidase enzyme in an amount sufficient to result in an increase in the dispersion of the antibody during subcutaneous administration. The hyaluronidase enzyme excipient in accordance with the formulation of the present invention is characterized by having no adverse effect on the molecular integrity of the bispecific EGFR/c-Met antibody in the stable pharmaceutical compositions described herein. Furthermore, the hyaluronidase enzyme merely modifies the delivery of the bispecific EGFR/c-Met antibody to the systemic circulation but does not possess any properties that could provide or contribute to the therapeutic effects of systemically absorbed bispecific EGFR/c-Met antibody. The hyaluronidase enzyme is not systemically bioavailable and does not adversely affect the molecular integrity of the bispecific EGFR/c-Met antibody at the recommended storage conditions of the stable pharmaceutical composition in accordance with the invention. A number of suitable hyaluronidase enzymes in accordance with the present invention are known. The preferred enzyme is a human hyaluronidase enzyme, such as a soluble human PH20 hyaluronidase, preferably the recombinant human hyaluronidase enzyme product known as rHuPH20. The amino acid sequence of soluble human PH20 hyaluronidases include the soluble human PH20 known as rHuPH20 and available under CAS Registry No. 757971-58-7. Soluble human PH20 hyaluronidiases are described in Int. Pat. Publ. No. WO2004/078140 and U.S. Pat. No. 7,767,429 incorporated herein by reference, in its entirety. In some embodiments, soluble hyaluronidases include those whose sequence are set forth in any of SEQ ID NOs: 21-25. Soluble PH20 hyaluronidase, when expressed in a cell, include a signal sequence for trafficking in the cell. Thus, in some embodiments, the amino acid sequence of a soluble PH20 hyaluronidase comprises SEQ ID NO: 26. In some embodiments, the amino acid sequence of sequence of a soluble PH20 hyaluronidase SEQ ID NO: 22, namely residues 36-482 of wild type human hyaluronidase. In some embodiments, the amino acid sequence of a soluble PH20 hyaluronidase comprises SEQ ID NO: 23. In some embodiments, the amino acid sequence of a soluble PH20 hyaluronidase comprises SEQ ID NO: 24. In some embodiments, the amino acid sequence a soluble PH20 hyaluronidase rHuPH20 comprises SEQ ID NO: 25. In some embodiments, the amino acid sequence of a soluble PH20 hyaluronidase comprises SEQ ID NO: 21. In some embodiments, the soluble PH20 hyaluronidases, when expressed in a cell, comprise a mixture of species that can include any one or more of SEQ ID NO: 21 to SEQ ID NO: 25 in various abundance. The average molecular weight is 61 kDa.

rHuPH20 refers to the composition produced upon expression in a cell, such as CHO cell, of nucleic acid encoding residues 36-482 of SEQ ID NO: 26, generally linked to the native or a heterologous signal sequence (residues 1-35 of SEQ ID NO: 26). rHuPH20 is produced by expression of a nucleic acid molecule, such as encoding amino acids 1-482 (set forth in SEQ ID NO: 26) in a mammalian cell. Translational processing removes the 35 amino acid signal sequence. As produced in the culture medium there is heterogeneity at the C-terminus such that the product, designated rHuPH20, includes a mixture of species that can include any one or more of the polypeptides 36-480, 36-481, and 36-482 of SEQ ID NO: 26, and some shorter polypeptides, in various abundance. Typically, rHuPH20 is produced in cells, such as CHO cells, for example DG44 CHO cells) that facilitate correct N-glycosylation to retain activity.

In some embodiments, the stable aqueous pharmaceutical composition comprises rHuPH20 at a concentration of about 500 U/mL, about 750 U/mL, about 1,000 U/mL, about 1,250 U/mL, about 1,500 U/mL, about 1,750 U/mL, about 2,000 U/mL, about 2,250 U/mL, about 2,500 U/mL, about 2,750 U/mL, about 3,000 U/mL, about 3,250 U/mL, about 3,500 U/mL, about 3,750 U/mL, or about 4,000 U/mL. In some embodiments, the stable aqueous pharmaceutical composition comprises rHuPH20 at a concentration of about 2,000 U/mL. In some embodiments, the stable aqueous pharmaceutical composition comprises rHuPH20 at a concentration of about 1,500 U/mL. According to some embodiments, the stable aqueous pharmaceutical composition comprises rHuPH20 at a concentration of about 0.005 mg/mL, about 0.0075 mg/mL, about 0.01 mg/mL, about 0.0125 mg/mL, about 0.015 mg/mL, about 0.0175 mg/mL, about 0.02 mg/mL, about 0.0225 mg/mL, about 0.025 mg/mL, about 0.0275 mg/mL, about 0.03 mg/mL, about 0.0325 mg/mL, about 0.035 mg/mL, about 0.0375 mg/mL, or about 0.04 mg/mL. In some embodiments, the stable aqueous pharmaceutical composition comprises rHuPH20 at a concentration of about 0.02 mg/ml.

In some embodiments, the stable aqueous pharmaceutical composition comprises rHuPH20 at a dose of about 10,000 U, about 11,000 U, about 12,000 U, about 13,000 U, about 13,200 U, about 14,000 U, about 15,000 U, about 16,000 U, about 17,000 U, about 17,500 U, about 18,000 U, about 19,000 U, about 19,500 U, about 19,600 U, about 19,680 U, about 19,700 U, about 20,000 U, about 21,000 U, about 22,000 U, about 23,000 U, about 24,000 U, about 25,000 U, about 26,000 U, about 26,260 U, about 26,500 U, about 26,600 U, about 26,680 U, about 26,700 U, about 27,000 U, 28,000 U, about 29,000 U, about 30,000 U or any value in between. In some embodiments, the stable aqueous pharmaceutical composition comprises rHuPH20 at a dose of about 13,000 U. In some embodiments, the stable aqueous pharmaceutical composition comprises rHuPH20 at a dose of about 13,200 U. In some embodiments, the stable aqueous pharmaceutical composition comprises rHuPH20 at a dose of about 14,000 U. In some embodiments, the stable aqueous pharmaceutical composition comprises rHuPH20 at a dose of about 17,500 U. In some embodiments, the stable aqueous pharmaceutical composition comprises rHuPH20 at a dose of about 18,000 U. In some embodiments, the stable aqueous pharmaceutical composition comprises rHuPH20 at a dose of about 19,680 U. In some embodiments, the stable aqueous pharmaceutical composition comprises rHuPH20 at a dose of about 20,000 U. In some embodiments, the stable aqueous pharmaceutical composition comprises rHuPH20 at a dose of about 26,000 U. In some embodiments, the stable aqueous pharmaceutical composition comprises rHuPH20 at a dose of about 26,260 U. In some embodiments, the stable aqueous pharmaceutical composition comprises rHuPH20 at a dose of about 26,300 U. In some embodiments, the stable aqueous pharmaceutical composition comprises rHuPH20 at a dose of about 26,400 U. In some embodiments, the stable aqueous pharmaceutical composition comprises rHuPH20 at a dose of about 26,500 U. In some embodiments, the stable aqueous pharmaceutical composition comprises rHuPH20 at a dose of about 26,600 U. In some embodiments, the stable aqueous pharmaceutical composition comprises rHuPH20 at a dose of about 27,000 U. In some embodiments, the stable aqueous pharmaceutical composition comprises rHuPH20 at a dose of about 27,500 U. In some embodiments, the stable aqueous pharmaceutical composition comprises rHuPH20 at a dose of about 28,000 U. In some embodiments, the stable aqueous pharmaceutical composition comprises rHuPH20 at a dose of about 28,500 U. In some embodiments, the stable aqueous pharmaceutical composition comprises rHuPH20 at a dose of about 29,000 U. In some embodiments, the stable aqueous pharmaceutical composition comprises rHuPH20 at a dose of about 30,000 U.

In some embodiments, the stable aqueous pharmaceutical compositions of the bispecific EGFR/c-Met antibody comprise a histidine and/or a pharmaceutically acceptable histidine salt, sucrose, polysorbate 80 (PS80), methionine, EDTA, a pH from about 5.2 to about 6.2, and, optionally, a hyaluronidase. The stable aqueous pharmaceutical compositions may comprise about 128 mg/mL to about 192 mg/mL of the bispecific EGFR-cMet antibody, about 10 mM to about 50 mM of histidine and/or a pharmaceutically acceptable histidine salt, about 6.8% (w/v) to about 10.2% (w/v) of sucrose, about 0.036% (w/v) to about 0.084% (w/v) of polysorbate 80 (PS80), about to 0.8 mg/mL to about 1.2 mg/mL of methionine, about 16 μg/mL to about 24 μg/mL of EDTA, and a pH from about 5.2 to about 6.2. The stable aqueous pharmaceutical compositions may optionally comprise about 1,000 U/mL to about 3,000 U/mL hyaluronidase. In some embodiments, the stable aqueous pharmaceutical compositions comprise about 160 mg/mL of the bispecific EGFR-cMet antibody, about 10 mM of histidine and/or a pharmaceutically acceptable histidine salt, about 8.5% (w/v) of sucrose, about 0.06% (w/v) of polysorbate 80 (PS80), about 1 mg/mL of methionine, about 20 μg/mL of EDTA, and a pH of about 5.7, and optionally about 2,000 U/mL hyaluronidase.

In other embodiments, the stable aqueous pharmaceutical compositions of the bispecific EGFR/c-Met antibody comprise acetate and/or a pharmaceutically acceptable acetate salt, sucrose, polysorbate 80 (PS80), methionine, EDTA, a pH from about 5.2 to about 6.2, and, optionally, a hyaluronidase. The stable aqueous pharmaceutical compositions may comprise about 128 mg/mL to about 192 mg/mL of the bispecific EGFR-cMet antibody, about 10 mM to about 50 mM of acetate and/or a pharmaceutically acceptable acetate salt, about 6.8% (w/v) to about 10.2% (w/v) of sucrose, about 0.036% (w/v) to about 0.084% (w/v) of polysorbate 80 (PS80), about to 0.8 mg/mL to about 1.2 mg/mL of methionine, about 16 μg/mL to about 24 μg/mL of EDTA, and a pH from about 5.2 to about 6.2. The stable aqueous pharmaceutical compositions may optionally comprise about 1,000 U/mL to about 3,000 U/mL hyaluronidase. In some embodiments, the stable aqueous pharmaceutical compositions comprise about 160 mg/mL of the bispecific EGFR-cMet antibody, about 30 mM of acetate and/or a pharmaceutically acceptable acetate salt, about 8.5% (w/v) of sucrose, about 0.06% (w/v) of polysorbate 80 (PS80), about 1 mg/mL of methionine, about 20 μg/mL of EDTA, and a pH of about 5.7, and optionally about 2,000 U/mL hyaluronidase.

Further provided herein are methods of treating cancer in a subject in need thereof by administering to the subject a therapeutically effective amount of a stable, aqueous pharmaceutical composition of the bispecific EGFR/c-Met antibody disclosed herein. The cancer may be a solid malignancy such as breast cancer (BC), prostate cancer, ovarian cancer (OC), cervical cancer, skin cancer, pancreatic cancer, gastroesophageal cancer (GEC), colorectal cancer (CRC), renal cell cancer (RCC), liver cancer, hepatocellular cancer (HCC), brain cancer, squamous cell carcinoma of the head and neck (SCCHN)), lymphoma, leukemia, lung cancer (e.g. non-small cell lung cancer (NSCLC) or small cell lung cancer (SCLC)), medullary thyroid cancer (MTC), and mesothelioma. According to some aspects, the solid malignancy may be metastatic or unresectable. In some embodiments, the solid malignancy is histologically or cytologically confirmed. The disclosed stable aqueous pharmaceutical compositions may be subcutaneously administered, for example, through a subcutaneous injection. The subcutaneous injection can be performed at various locations of the subject's body such as but not limited to the upper arm, the thigh, the abdomen or the lower back.

In one embodiment the cancer is a lung cancer. In one embodiment the cancer is a non-small cell lung cancer (NSCLC). In one embodiment the cancer is treatment-naïve locally advanced or metastatic NSCLC. In one embodiment the cancer has been treated previously by IV amivantamab. In one embodiment the cancer is harboring an EGFR exon 19del mutation. In one embodiment the cancer is harboring exon 21 L858R mutation. In one embodiment the cancer is harboring an EGFR exon20ins mutation. In one embodiment the cancer has experienced disease progression on or after treatment with a third-generation EGFR tyrosine kinase inhibitor (TKI).

Further provided herein are methods of reducing infusion-related reactions in a subject treated with amivantamab comprising subcutaneously administering to the subject the stable aqueous pharmaceutical formulation as disclosed herein. The subject is in need of treatment for a cancer as disclosed herein.

Further provided herein are articles of manufacture containing a stable, aqueous pharmaceutical composition of the invention. In one embodiment, the article of manufacture is a single-use glass vial equipped with a stopper, which contains the stable, aqueous pharmaceutical composition to be administered. In some embodiments, the stopper is pierceable by a syringe. In some embodiments, the vial is sealed. In some embodiments, the single-use vial is a 10 mL single-use glass vial with a 20 mm stopper, covered by a 20 mm aluminum seal. In some embodiments, the vial size is 2R, 4R, 6R, 8R, 10R, 15R, 20R, 25R, 30R, or 50R ISO format with a capacity of about: 4 mL, 6 mL, 10 mL, 12 mL, 14 mL, 20 ml, 26 mL, 33 mL, 38 mL, or 62 mL respectively. In one embodiment, the total volume of the stable aqueous pharmaceutical composition (also referred to herein as drug product or DP) ranges from about 5 mL to about 10 mL. In one embodiment, the total volume of the stable aqueous pharmaceutical composition (drug product or DP) ranges from about 0.5 mL to about 20 mL, from about 1 mL to about 15 mL, from about 5 mL to about 10 mL, or from about 6 mL to about 8 mL. In one embodiment, the total volume of the stable aqueous pharmaceutical composition is about: 0.5 mL, 0.6 mL, 0.7 mL, 0.8 mL, 0.9 mL, 1 mL, 2 mL, 3 mL, 4 mL, 5 mL, 6 mL, 6.5 mL, 6.6 mL, 6.7 mL, 7 mL, 7.1 mL, 8 mL, 8.5 mL, 8.6 mL, 8.7 mL, 8.75 mL, 8.8 mL, 9 mL, 10 mL, 11 mL, 12 mL, 13 mL, 14 mL, 15 mL, 16 mL, 18 mL, 19 mL, 20 mL, 25 mL, or 30 mL or any ranges there in between.

### Drug Product Stability

In some embodiments, the DP stability is determined following storage for a specified period of time. In some embodiments, DP is stored for about 3 months or more, about 6 months or more, about 12 months or more, about 1.5 years or more, about 2 years or more, about 2.5 years or more, about 3 years or more, about 3.5 years or more, about 4 years or more, about 4.5 years or more, about 5 years or more, about 6 years or more, about 7 years or more, about 8 years or more, about 9 years or more, or about 10 years or more. In some embodiments, DP is stored for about 12 months or more, about 1.5 years or more, about 2 years or more, about 2.5 years or more, or about 3 years or more. In some embodiments, DP is stored for about 2 years or more.

### Temperature

In some embodiments, the DP is stable following storage at a specific temperature for a specified period of time. In some embodiments, the temperature ranges between about: −10 to 50° C., 0 to 25° C., 1 to 20° C., 1 to 15° C., 2 to 10° C., or 2 to 5° C. In some embodiments, the temperature ranges between about: 2 to 8° C. In some embodiments, the temperature is about: −10° C., −9° C., −8° C., −7° C., −6° C., −5° C., −4° C., −3° C., −2° C., −1° C., 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C. or 50° C.

In some embodiments, the DP is stable following storage for about 12 months or more, or for about 2 years or more and at a temperature ranging from about 2° C. to about 8° C. In some embodiments, the DP is stable following storage for about 12 months or more or for about 2 years or more and at a temperature of about 5° C. In some embodiments, the DP is stable following storage for about 12 months or more and at a temperature of about 25° C.

The stability of the presently disclosed aqueous pharmaceutical compositions, also referred to as drug product (DP), is determined based on specific amount or proportion of the bispecific EGFR-cMet antibody and other constituents of the DP as provided herein (such as, but not limited to, buffering agents, stabilizers, chelating agents, surfactants, and enzymes), as well as the assessment of various factors. These factors include but are not limited to the color of the solution, the pH, the turbidity, number of subvisible particles, percentage of aglycosylated heavy chain (AGHC), percentage of new peak(s), percentage of high molecular weight species (HMWS), percentage of low molecular weight species (LMWS), percentage of sum of acidic peaks, percentage of sum of basic peaks, protein concentration, percentage of EGFR binding activity, percentage of cMet binding activity, and/or percentage of PS80.

Stable DP as disclosed herein should not be construed to require all the factors listed herein but rather at least one, at least two, or at least three or more of those factors. In some embodiments, the stable disclosed DP exhibits the following results for at least one, at least two, at least three or more of the factors listed in detail below herein. In some embodiments, the stable DP exhibits the following results for all the factors listed in detail below herein.

### Color of Solution

The Color of a DP solution is monitored and can be assessed to verify that the appearance of the solution is consistent with previous batches at release and over the shelf life. The color of the DP solution can reflect stability. In one embodiment, the stability of the DP is defined when having a color of solution spanning from colorless to about BY2 or less, to about BY4 or less, to about B2 or less, to about B4 or less, to about Y2 or less or to about Y4 or less as described in the European Pharmacopoeia 2.2.2, Degree of Coloration of Liquids European Pharmacopoeia (Ph. Eur.) 10th Edition monograph number 20202, July 2019.

In one embodiment, the stability is defined as having a color of solution of colorless to about BY2 or less, about B2 or less, about Y2 or less after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a color of solution of colorless to about BY4 or less, to about B4 or less, to about Y4 or less after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a color of solution of colorless to about BY5 or less, to about B5 or less, to about Y5 or less after storage for about 12 months or more and at a temperature of about 5° C., after storage for about 12 months or more and at a temperature of about 25° C., and/or after storage for about 2 years or more and at a temperature of about 5° C.

pH

Measuring the pH of the DP solution allows confirmation that it is consistent with previous DP batches at release and over the shelf life. In one embodiment, the stability of the DP is defined when its pH is about 5.0, 5.1, 5.2, 5.3, 5.4, 5.5, 5.6, 5.7, 5.8, 5.9, 6.0, 6.1, 6.2, 6.3, or 6.4. In one embodiment, the pH of the DP is about 5.7 after storage for about 12 months or more and at a temperature of about 5° C., after storage for about 12 months or more and at a temperature of about 25° C., and/or after storage for about 2 years or more and at a temperature of about 5° C. In one embodiment, stability is defined as having a pH range from about 5.0 to about 6.4 after storage for about 12 months or more and at a temperature of about 5° C., after storage for about 12 months or more and at a temperature of about 25° C., and/or after storage for about 2 years or more and at a temperature of about 5° C. In a preferred embodiment, the stability of the DP is defined when its pH ranges from about 5.2 to about 6.2 after storage for about 12 months or more and at a temperature of about 5° C., after storage for about 12 months or more and at a temperature of about 25° C., and/or after storage for about 2 years or more and at a temperature of about 5° C. In a most preferred embodiment, the stability of the DP is defined when its pH ranges from about 5.4 to about 6.0 after storage for about 12 months or more and at a temperature of about 5° C., after storage for about 12 months or more and at a temperature of about 25° C., and/or after storage for about 2 years or more and at a temperature of about 5° C.

### Turbidity

Turbidity allows measuring the presence of particles in the DP solution in order to ensure consistency with previous DP batches and applicable compendia guidance at release and over the shelf life. Test results are reported in nephelometric turbidity units (NTU). In one embodiment, the stability of the DP is defined when its turbidity value is about: 1, 2, 3, 4, 5, 6, 7, 8, 9, 10, 11, 12, 13, 14, 15, 16, 17, 18, 19, or 20 nephelometric turbidity units (NTU) after storage for about 12 months or more and at a temperature of about 5° C., after storage for about 12 months or more and at a temperature of about 25° C., and/or after storage for about 2 years or more and at a temperature of about 5° C. In one embodiment, the stability of the DP is defined as having a turbidity value of about 18 NTU or less after storage for about 12 months or more and at a temperature of about 5° C., after storage for about 12 months or more and at a temperature of about 25° C., and/or after storage for about 2 years or more and at a temperature of about 5° C. In a preferred embodiment, the stability of the DP is defined when as a turbidity value of about 13 NTU or less after storage for about 12 months or more and at a temperature of about 5° C., after storage for about 12 months or more and at a temperature of about 25° C., and/or after storage for about 2 years or more and at a temperature of about 5° C. In the most preferred embodiment, the stability of the DP is defined as having a turbidity value of about 8 NTU or less after storage for about 12 months or more and at a temperature of about 5° C., after storage for about 12 months or more and at a temperature of about 25° C., and/or after storage for about 2 years or more and at a temperature of about 5° C.

### Particle Analysis

The stability of the DP is set to a specific threshold of particles contamination based on the average number of sub-visible particles. Testing results are to comply with United States Pharmacopoeia <788> Particulate Matter, European Pharmacopoeia 2.9.19, and Japanese Pharmacopoeia XVII/6.07 Particulate Contamination: Sub-visible particles. As such, the average number of particles present in the DP units tested should not exceed 6000 particles per container for particle size equal to 10 μm or greater and should not exceed 600 particles per container for particle size equal to 25 μm or greater.

cSDS Conditions

Capillary SDS-PAGE (cSDS), like gel-based SDS-PAGE, is a method for separating denatured protein based on molecular weight. This process allows quantifying DP purity and monitoring its stability at release and over the shelf life.

In one embodiment, the DP stability is defined based upon various results of cSDS variables (e.g. percent purity, aglycosylated heavy chain (AGHC), or presence of new peak) where the cSDS was performed under reduced or non-reduced conditions after storage for about 12 months or more and at a temperature of about 5° C., after storage for about 12 months or more and at a temperature of about 25° C., and/or after storage for about 2 years or more and at a temperature of about 5° C.

cSDS Reduced results consistent with stability. In one embodiment, stability is defined as having a percent purity greater than or equal to 88.0%, AGHC less than or equal to 11.0%, and no new peak greater than 1.5% compared to a validated stock of amivantamab Reference Material after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined a percent purity about or more than 91.0%, AGHC less than or about 8.0%, and no new peak more than 1.0% compared to Reference Material after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a percent purity about or more than 94.0%, AG HC less than or about 5.0%, and no new peak more than 1.0% compared to Reference Material after storage for about 12 months or more and at a temperature of about 5° C., after storage for about 12 months or more and at a temperature of about 25° C., and/or after storage for about 2 years or more and at a temperature of about 5° C.

cSDS Non-Reduced results consistent with stability. In one embodiment, stability is defined as having a percent purity of about 88.0% or more and no new peak more than 1.5% compared to Reference Material after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as percent purity of about 90.0% or more and no new peak more than 1.0% compared to Reference Material after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a percent purity of about 94.0% or more and no new peak more than 1.0% compared to Reference Material after storage for about 12 months or more and at a temperature of about 5° C., after storage for about 12 months or more and at a temperature of about 25° C., and/or after storage for about 2 years or more and at a temperature of about 5° C.

In one embodiment, the DP stability is defined as having a percent purity about: 80%, 81%, 82%, 83%, 84%, 85%, 86%, 87%, 88%, 89%, 90%, 91%, 92%, 93%, 94%, 95%, 96%, 97%, 98%, 99% or about or equal to 100% or any range there in between.

In one embodiment, the DP stability is defined as having an AGHC of about: 2%, 3%, 4%, 5%, 6%, 7%, 8%, 9%, 10%, 11%, 12%, 13%, 14% or any range there in between.

In one embodiment, the DP stability is defined as showing no new peak in the cSDS results of more than 0.5%, 0.8%, 0.9%, 1.0%, 1.2%, 1.3%, 1.4%, 1.5%, 1.6%, 1.7%, 1.8%, 1.9% or more than 2% when compared to an untreated Reference Material.

Size-Exclusion HPLC (SE-HPLC) Results Consistent with Stability

SE-HPLC procedure allows assessing purity of the DP and monitoring its stability under non-denaturing conditions at release and over the shelf life.

SE-HPLC Results Consistent with Stability

Main Component—In one embodiment, stability is defined as having a Main Component about 90.0% or more after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a Main Component about 95.0% or more after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a Main Component about 97.0% after storage for about 12 months or more and at a temperature of about 5° C., after storage for about 12 months or more and at a temperature of about 25° C., and/or after storage for about 2 years or more and at a temperature of about 5° C. High Molecular Weight Species (HMWS)—In one embodiment, stability is defined as having a HMWS of about 10.0% or less after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a HMWS of about 5.0% or less after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a HMWS of about 3.0% or less after storage for about 12 months or more and at a temperature of about 5° C., after storage for about 12 months or more and at a temperature of about 25° C., and/or after storage for about 2 years or more and at a temperature of about 5° C. Low Molecular Weight Species (LMWS)—In one embodiment, stability is defined as having a LMWS about 5.0% or less after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a LMWS of about 2.0% or less after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a LMWS about 1.0% or less after storage for about 12 months or more and at a temperature of about 5° C., after storage for about 12 months or more and at a temperature of about 25° C., and/or after storage for about 2 years or more and at a temperature of about 5° C.

Capillary Isoelectric Focusing (cIEF)

The cIEF, like isoelectric gel electrophoresis (IEF) methods, separates proteins on the basis of overall charge or isoelectric point (pI). This procedure allows monitoring the distribution of charge-based isoforms of the drug product at release and over the shelf life In one embodiment, the DP stability is defined based upon various results of cIEF variables such as the Main Peak (MP), the sum of acidic peaks or the sum of basic peaks, after DP storage for about 12 months or more and at a temperature of about 25° C., and/or after storage for about 2 years or more and at a temperature of about 5° C.

cIEF Results Consistent with Stability

### Main Peak—

In one embodiment, the DP stability is defined as having a cIEF with a MP of about: 30%, 35%, 40%, 45%, 50%, 55%, 60%, 65%, 70%, 75%, 80%, 85%, 90% or 95% or any range there in between after DP storage for about 12 months or more and at a temperature of about 25° C., and/or after storage for about 2 years or more and at a temperature of about 5° C. In one embodiment, the DP stability is defined as having a cIEF with a MP ranging from about 30% to about 90% after DP storage for about 12 months or more and at a temperature of about 25° C., and/or after storage for about 2 years or more and at a temperature of about 5° C. In one embodiment, stability is defined as having a Main Peak of 37-87% after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a Main Peak of 47-87% after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a Main Peak of 57-87% after storage for about 12 months or more and at a temperature of about 5° C., after storage for about 12 months or more and at a temperature of about 25° C., and/or after storage for about 2 years or more and at a temperature of about 5° C.

### Sum of Acidic Peaks—

In one embodiment, the DP stability is defined as having a cIEF with a with a sum of acidic peaks totaling to about: 5%, 10%, 15%, 20%, 25%, 30%, 35%, 40%, 45%, 50%, 55%, 60%, 65% or 70% or any range there in between after DP storage for about 12 months or more and at a temperature of about 25° C., and/or after storage for about 2 years or more and at a temperature of about 5° C. In one embodiment, stability is defined as having a Sum of acidic peaks totaling 10-60% after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a Sum of acidic peaks totaling 10-50% after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a Sum of acidic peaks totaling 10-40% after storage for about 12 months or more and at a temperature of about 5° C., after storage for about 12 months or more and at a temperature of about 25° C., and/or after storage for about 2 years or more and at a temperature of about 5° C.

### Sum of Basic Peaks—

In one embodiment, the DP stability is defined as having a cIEF with a sum of basic peaks totaling about: 1%, 2%, 3%, 4%, 5%, 6%, 7%, 8%, 9%, 10%, 11%, 12%, 13%, 14% or 15% or any range there in between after DP storage for about 12 months or more and at a temperature of about 25° C., and/or after storage for about 2 years or more and at a temperature of about 5° C. In one embodiment, stability is defined as having a Sum of basic peaks totaling about 12.0% or less after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having a Sum of basic peaks totaling about 10.0% or less after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having a Sum of basic peaks totaling about 8.0% or less after storage for about 12 months or more and at a temperature of about 5° C., after storage for about 12 months or more and at a temperature of about 25° C., and/or after storage for about 2 years or more and at a temperature of about 5° C.

### Protein Concentration by A280

Protein concentration of the DP allows verifying that it is consistent with previous DP batches at release and over the shelf life. Quantification of protein concentration can be accomplished by measuring the UV light absorbance of the drug product solution at 280 nm (A280).

Protein concentration results consistent with stability of DP. In one embodiment, the DP stability is defined as having a protein concentration of 128 to 192 mg/mL after storage for about 12 months or more and at a temperature of about 5° C., after storage for about 12 months or more and at a temperature of about 25° C., and/or after storage for about 2 years or more and at a temperature of about 5° C. In a preferred embodiment, DP stability is defined as having a protein concentration of 144 to 176 mg/mL after storage for about 12 months or more and at a temperature of about 5° C., after storage for about 12 months or more and at a temperature of about 25° C., and/or after storage for about 2 years or more and at a temperature of about 5° C. In the most preferred embodiment, DP stability is defined as having a protein concentration of 150 mg/mL to 170 mg/mL after storage for about 12 months or more and at a temperature of about 25° C., and/or after storage for about 2 years or more and at a temperature of about 5° C.

### Drug Product Potency

In vitro binding of the DP to EGFR and/or c-Met allows assessing the level of DP stability. This binding can be assessed by using, but not limited to, a homogeneous competitive time resolved fluorescence resonance energy transfer (TR-FRET) assay.

EGFR Binding Activity Results Consistent with Stability.

In one embodiment, the DP stability is defined as having an EGFR binding activity, relative to a reference, of about: 40%, 50%, 60%, 70%, 80%, 90%, 100%, 110%, 120%, 130%, 140%, 150%, 160% or 170% or any range there in between after DP storage for about 12 months or more and at a temperature of about 5° C., after storage for about 12 months or more and at a temperature of about 25° C., and/or after storage for about 2 years or more and at a temperature of about 5° C. In one embodiment, the DP stability is defined as having an EGFR binding activity ranging from about 50% to about 150% relative to a reference after DP storage for about 12 months or more and at a temperature of about 25° C., and/or after storage for about 2 years or more and at a temperature of about 5° C. In a preferred embodiment, the DP stability is defined as having an EGFR binding activity ranging from about 60% to about 140% relative to a reference after DP storage for about 12 months or more and at a temperature of about 25° C., and/or after storage for about 2 years or more and at a temperature of about 5° C. In a most preferred embodiment, the DP stability is defined as having an EGFR binding activity ranging from about 80% to about 120% relative to a reference after DP storage for about 12 months or more and at a temperature of about 25° C., and/or after storage for about 2 years or more and at a temperature of about 5° C.

cMet Binding Activity Results Consistent with Stability.

In one embodiment, the DP stability is defined as having a cMet binding activity, relative to a reference, of about: 40%, 50%, 60%, 70%, 80%, 90%, 100%, 110%, 120%, 130%, or 140% or any range there in between after DP storage for about 12 months or more and at a temperature of about 25° C., and/or after storage for about 2 years or more and at a temperature of about 5° C. In one embodiment, the DP stability is defined as having a cMet binding activity ranging from about 50% to about 150% relative to a reference after DP storage for about 12 months or more and at a temperature of about 25° C., and/or after storage for about 2 years or more and at a temperature of about 5° C. In a preferred embodiment, the DP stability is defined as having a cMet binding activity ranging from about 60% to about 140% relative to a reference after DP storage for about 12 months or more and at a temperature of about 25° C., and/or after storage for about 2 years or more and at a temperature of about 5° C. In a most preferred embodiment, the DP stability is defined as having a cMet binding activity ranging from about 80% to about 120% relative to a reference after DP storage for about 12 months or more and at a temperature of about 25° C., and/or after storage for about 2 years or more and at a temperature of about 5° C.

Potency rHuPH20 Activity

In vitro rHuPH20 hyaluronidase enzymatic activity is determined by measuring turbidity when hyaluronic acid (HA), a substrate of rHuPH20, binds with acidified serum. The determination of hyaluronidase activity is based on the formation of a precipitate when hyaluronic acid (HA) binds with acidified serum. The activity is measured by incubating hyaluronidase with HA for 30 minutes in a 96-well plate format at 37° C. and then precipitating the undigested HA with the addition of acidified serum. The resulting turbidity is measured at 640 nm and the decrease in turbidity resulting from enzymatic cleavage of the HA substrate is a measure of the hyaluronidase activity.

rHuPH20 Activity Results Consistent with Stability.

In one embodiment, stability is defined as having rHuPH20 activity of about: 800 U/mL, 900 U/mL, 1000 U/mL, 1100 U/mL, 1200 U/mL, 1300 U/mL, 1400 U/mL, 1500 U/mL, 1600 U/mL, 1700 U/mL, 1800 U/mL, 1900 U/mL, 2000 U/mL, 2100 U/mL, 2200 U/mL, 2300 U/mL, 2400 U/mL, 2500 U/mL, 2600 U/mL, 2700 U/mL, 2800 U/mL, 2900 U/mL, 3000 U/mL, 3100 U/mL, 3200 U/mL, 3300 U/mL, 3400 U/mL or 3500 U/mL or any range there in between after storage for about 12 months or more and at a temperature of about 5° C., after storage for about 12 months or more and at a temperature of about 25° C., and/or after storage for about 2 years or more and at a temperature of about 5° C. In one embodiment, stability is defined as having rHuPH20 activity of 1000 U/mL to 3000 U/mL after storage for about 12 months or more and at a temperature of about 5° C., after storage for about 12 months or more and at a temperature of about 25° C., and/or after storage for about 2 years or more and at a temperature of about 5° C. In a preferred embodiment, stability is defined as having rHuPH20 activity of 1500 U/mL to 2500 U/mL after storage for about 12 months or more and at a temperature of about 5° C., after storage for about 12 months or more and at a temperature of about 25° C., and/or after storage for about 2 years or more and at a temperature of about 5° C. In the most preferred embodiment, stability is defined as having rHuPH20 activity of 1800 U/mL to 2200 U/mL after storage for about 12 months or more and at a temperature of about 5° C., after storage for about 12 months or more and at a temperature of about 25° C., and/or after storage for about 2 years or more and at a temperature of about 5° C.

### Analytical Tests—Surfactant

### Polysorbate-80 Quantification

Polysorbate 80 is quantitatively determined by mixed-mode ion-exchange/hydrophobic HPLC. In one embodiment, the DP stability is defined by a PS80 concentration in percentage weight to volume of about: 0.02%, 0.03%, 0.04%, 0.05%, 0.06%, 0.08%, 0.09% or 0.1% or any range there in between after DP storage for about 12 months or more and at a temperature of about 5° C., after storage for about 12 months or more and at a temperature of about 25° C., and/or after storage for about 2 years or more and at a temperature of about 5° C. In one embodiment, DP stability is defined as a PS80 concentration of 0.03-0.08% after storage for about 12 months or more and at a temperature of about 5° C., after storage for about 12 months or more and at a temperature of about 25° C., and/or after storage for about 2 years or more and at a temperature of about 5° C. In a preferred embodiment, DP stability is defined as a PS 80 concentration of 0.04-0.08% after storage for about 12 months or more and at a temperature of about 5° C., after storage for about 12 months or more and at a temperature of about 25° C., and/or after storage for about 2 years or more and at a temperature of about 5° C. In the most preferred embodiment, DP stability is defined as a PS 80 concentration of 0.05-0.08% after storage for about 12 months or more and at a temperature of about 5° C., after storage for about 12 months or more and at a temperature of about 25° C., and/or after storage for about 2 years or more and at a temperature of about 5° C.

### Viscosity

A potential problem with using high concentration protein formulations, recognized by those skilled in the art, is that the viscosity of the solution usually increases with the increase of the protein concentration. Viscous antibody solutions are difficult to process (e.g. fill vials and/or syringes) and to administer to patients. Highly viscous formulations are difficult to manufacture, draw into a syringe, and inject. The use of force in manipulating the viscous formulations leads to excessive frothing, which can lead to denaturation and inactivation of active biologics. Unless viscosity can be reduced, high concentration antibody formulations may require larger bore needles, high pressure injections, longer injection times, and special equipment or materials to counteract antibody adhesion. These changes will increase patient discomfort and the cost of manufacturing the therapeutic antibody product.

As used herein, “viscosity” is a fluid's resistance to flow, and may be measured in units of centipoise (cP) or milliPascal-second (mPa-s), where 1 cP=1 mPa-s, at a given shear rate. Viscosity may be measured by using a viscometer, e. g., Brookfield Engineering Dial Reading Viscometer, model LVT, and AR-G2, TA instruments. Viscosity may be measured using any other methods and in any other units known in the art (e. g. absolute, kinematic or dynamic viscosity), understanding that it is the percent reduction in viscosity afforded by use of the excipients described by the invention that is important. Regardless of the method used to determine viscosity, the percent reduction in viscosity in excipient formulations versus control formulations will remain approximately the same at a given shear rate.

The present invention provides a high concentration amivantamab composition with a viscosity that is between about 9 cP and about 11 cP at room temperature. In some embodiments, the high concentration amivantamab composition has an absolute viscosity of about 22 cP or less, 16 cP or less, 13 cP or less, 11 cP or less, 9 cP or less, 8 cP or less, 7 cP or less, 6 cP. In some embodiments, the high concentration amivantamab composition has a viscosity of about 21.9 cP as measured at 4° C. In some embodiments, the high concentration amivantamab composition has a viscosity of about 16 cP as measured at 10° C. In some embodiments, the high concentration amivantamab composition has a viscosity of about 13.3 cP as measured at 15° C. In some embodiments, the high concentration amivantamab composition has a viscosity of about 11 cP as measured at 20° C. In some embodiments, the high concentration amivantamab composition has a viscosity of about 9.3 cP as measured at 25° C. In some embodiments, the high concentration amivantamab composition has a viscosity of about 7.9 cP as measured at 30° C. In some embodiments, the high concentration amivantamab composition has a viscosity of about 6.7 cP as measured at 35° C. In some embodiments, the high concentration amivantamab composition has a viscosity of about 5.8 cP as measured at 40° C.

### Illustrative Embodiments

Provided here are illustrative embodiments of the disclosed technology. These embodiments are illustrative only and do not limit the scope of the present disclosure or of the claims attached hereto.
1. A stable aqueous pharmaceutical composition comprising a bispecific epidermal growth factor receptor (EGFR)/hepatocyte growth factor receptor (c-Met) antibody and a hyaluronidase, wherein the antibody comprises:
   a. a first heavy chain (HC1) comprising a HC1 variable region 1 (VH1) comprising the amino acid sequence of SEQ ID NO:13;
   b. a first light chain (LC1) comprising a light chain variable region 1 (VL1) comprising the amino acid sequence of SEQ ID NO:14;
   c. a second heavy chain (HC2) comprising a HC2 variable region 2 (VH2) comprising the amino acid sequence of SEQ ID NO:15;
   d. a second light chain (LC2) comprising a light chain variable region 2 (VL2) comprising the amino acid sequence of SEQ ID NO:16;
and wherein the composition comprises about 1,050 mg to about 2,240 mg of the bispecific EGFR/c-Met antibody and about 13,000 U to about 28,000 U of the hyaluronidase.
2. The stable composition of embodiment 1 wherein the composition comprises about 1,050 mg of the bispecific EGFR/c-Met antibody.
3. The stable composition of embodiment 1 wherein the composition comprises about 1,400 mg of the bispecific EGFR/c-Met antibody.
4. The stable composition of embodiment 1 wherein the composition comprises about 1,575 mg of the bispecific EGFR/c-Met antibody.
4a. The stable composition of embodiment 1 wherein the composition comprises about 1,600 mg of the bispecific EGFR/c-Met antibody.
5. The stable composition of embodiment 1 wherein the composition comprises about 2,100 mg of the bispecific EGFR/c-Met antibody.
5a. The stable composition of embodiment 1 wherein the composition comprises about 2,240 mg of the bispecific EGFR/c-Met antibody.
6. A stable aqueous pharmaceutical composition comprising:
   a) about 144 mg/mL to about 176 mg/mL of a bispecific epidermal growth factor receptor (EGFR)/hepatocyte growth factor receptor (c-Met) antibody, the bispecific antibody comprising:
      a first heavy chain (HC1) comprising a HC1 variable region 1 (VH1);
      a first light chain (LC1) comprising a light chain variable region 1 (VL1);
      a second heavy chain (HC2) comprising a HC2 variable region 2 (VH2); and
      a second light chain (LC2) comprising a light chain variable region 2 (VL2),
      wherein the VH1 comprises a heavy chain complementarity determining region 1 (HCDR1), a HCDR2 and a HCDR3 amino acid sequences of SEQ ID NOs: 1, 2, and 3, respectively; the VL1 comprises a light chain complementarity determining region 1 (LCDR1), a LCDR2 and a LCDR3 amino acid sequences of SEQ ID NOs: 4, 5 and 6, respectively, the VH2 comprises the HCDR1, the HCDR2 and the HCDR3 amino acid sequences of SEQ ID NOs: 7, 8 and 9, respectively; and the VL2 comprises the LCDR1, the LCDR2 and the LCDR3 amino acid sequences of SEQ ID NOs: 10, 11 and 12, respectively;
   b) about 10 mM to about 50 mM of acetate and/or pharmaceutically acceptable acetate salt;
   c) about 6.8% (w/v) to about 10.2% (w/v) of sucrose;
   d) about 0.036% (w/v) to about 0.084% (w/v) of polysorbate 80 (PS80);
   e) about to 0.8 mg/mL to about 1.2 mg/mL of methionine;
   f) about 16 μg/mL to about 24 μg/mL of ethylenediaminetetraacetic acid (EDTA);
   g) optionally, about 1,000 U/mL to about 3,000 U/mL of hyaluronidase; and
   h) a pH from about 5.2 to about 6.2.
6a. The stable aqueous pharmaceutical composition of embodiment 6, wherein viscosity of the composition is less than about 11 cP as measured at 20° C.
7. The stable aqueous pharmaceutical composition of embodiment 6, wherein the bispecific EGFR-cMet antibody comprises an HC1 variable region comprising the amino acid sequence of SEQ ID NO:13 and a LC1 variable region comprising the amino acid sequence of SEQ ID NO:14.
8. The stable aqueous pharmaceutical composition of embodiment 6 or embodiment 7, wherein the bispecific EGFR-cMet antibody comprises a HC2 variable region comprising the amino acid sequence of SEQ ID NO:15 and a LC2 variable region comprising the amino acid sequence of SEQ ID NO:16.
9. The stable aqueous pharmaceutical composition of any one of embodiments 6-8, wherein the HC1 comprises the amino acid sequence of SEQ ID NO:17 and the LC1 comprises the amino acid sequence of SEQ ID NO:18.
10. The stable aqueous pharmaceutical composition of any one of embodiments 6-9, wherein the HC2 comprises the amino acid sequence of SEQ ID NO: 19 and the LC2 comprises the amino acid sequence of SEQ ID NO:20.
11. The stable aqueous pharmaceutical composition of any one of embodiments 6-10, wherein the bispecific EGFR-cMet antibody is amivantamab or a biosimilar thereof.
12. The stable aqueous pharmaceutical composition of any one of embodiments 6-11, wherein the bispecific EGFR-cMet antibody has a concentration of about 160 mg/mL.
13. The stable aqueous pharmaceutical composition of any one of embodiments 6-12, wherein the acetate and/or pharmaceutically acceptable acetate salt has a concentration of about 30 mM.
14. The stable aqueous pharmaceutical composition of any one of embodiments 6-13, wherein the acetate and/or pharmaceutically acceptable acetate salt comprises glacial acetic acid and/or sodium acetate trihydrate.
15. The stable aqueous pharmaceutical composition of any one of embodiments 6-14, comprising about 8.5% (w/v) sucrose.
16. The stable aqueous pharmaceutical composition of any one of embodiments 6-15, comprising about 0.06% (w/v) PS80.
17. The stable aqueous pharmaceutical composition of any one of embodiments 6-16, wherein the methionine comprises L-methionine and has a concentration of about 1 mg/mL.
18. The stable aqueous pharmaceutical composition of any one of embodiments 6-17, wherein the EDTA has a concentration of about 20 μg/mL.
19. The stable aqueous pharmaceutical composition of any one of embodiments 6-18, wherein the pH is about 5.7.
20. The stable aqueous pharmaceutical composition of any one of embodiments 6-19, wherein the hyaluronidase is a human hyaluronidase, optionally rHuPH20 comprising the amino acid sequence of SEQ ID NO: 21.
21. The stable aqueous pharmaceutical composition of any one of embodiments 6-20, wherein the concentration rHuPH20 is about 1,000 U/mL to about 3,000 U/mL.
22. The stable aqueous pharmaceutical composition of any one of embodiments 6-21, wherein the concentration rHuPH20 is about 2,000 U/mL.
23. The stable aqueous pharmaceutical composition of any one of embodiments 6-22, wherein stability is defined based on color of solution, pH, turbidity, number of subvisible particles, percentage of aglycosylated heavy chain (AGHC), percentage of new peak(s), percentage of high molecular weight species (HMWS), percentage of low molecular weight species (LMWS), percentage of sum of acidic peaks, percentage of sum of basic peaks, protein concentration, percentage of EGFR binding activity, percentage of cMet binding activity, percentage of PS80, optionally, percentage of rHuPH20 activity, or any combination thereof.
24. The stable aqueous pharmaceutical composition of any one of embodiments 6-23, wherein the total volume of the composition ranges from about 6 mL to about 9 mL.
25. The stable aqueous pharmaceutical composition of embodiment 24, wherein the total volume of the composition is about 7.1 mL.
26. The stable aqueous pharmaceutical composition of embodiment 24, wherein the total volume of the composition is about 6.6 mL.
27. The stable aqueous pharmaceutical composition of embodiment 24, wherein the total volume of the composition is about 8.75 mL.
28. The stable aqueous pharmaceutical composition of any one of embodiments 1-27, comprising about 160 mg/mL of the bispecific EGFR-cMet antibody, about 30 mM acetate and/or pharmaceutically acceptable acetate salt, about 8.5% sucrose, and about 1 mg/mL L-methionine with polysorbate 80 to a final concentration of about 0.06% (w/v) and EDTA to a final concentration of about 20 μg/mL, wherein the stable aqueous pharmaceutical composition has pH about 5.7,
   and wherein the bispecific EGFR-cMet antibody comprises a heavy chain 1 (HC1) comprising the amino acid sequence of SEQ ID NO:17, HC2 comprising the amino acid sequence of SEQ ID NO:19, a light chain 1 (LC1) comprising the amino acid sequence of SEQ ID NO:18, and a LC2 comprising the amino acid sequence of SEQ ID NO:20.
29. The stable aqueous pharmaceutical composition of any one of embodiments 1-27, comprising about 160 mg/mL of the bispecific EGFR-cMet antibody, about 30 mM acetate and/or pharmaceutically acceptable acetate salt, about 8.5% sucrose, about 1 mg/mL L-methionine with polysorbate 80 to a final concentration of about 0.06% (w/v) and EDTA to a final concentration of about 20 μg/mL, and rHuPH20 to a final concentration of about 2,000 U/mL, wherein the stable aqueous pharmaceutical composition has pH about 5.7, and wherein the bispecific EGFR-cMet antibody comprises a heavy chain 1 (HC1) comprising the amino acid sequence of SEQ ID NO:17, HC2 comprising the amino acid sequence of SEQ ID NO:19, a light chain 1 (LC1) comprising the amino acid sequence of SEQ ID NO:18, and a LC2 comprising the amino acid sequence of SEQ ID NO:20.
30. A method of treating cancer in a subject in need thereof, the method comprising administering to the subject the pharmaceutical composition of any one of embodiments 1-29.
31. The method of embodiment 30, wherein the administration is subcutaneous.
32. The method of embodiments 30-31, wherein the cancer comprises lung cancer, squamous cell carcinoma of the head and neck (SCCHN), hepatocellular cancer (HCC), colorectal cancer (CRC), renal cell cancer (RCC), medullary thyroid cancer (MTC), gastroesophageal cancer (GEC), mesothelioma, breast cancer (BC) or ovarian cancer (OC).
33. The method of embodiments 30-31, wherein the cancer comprises non-small cell lung cancer (NSCLC).
34. A method for preparing a stable aqueous pharmaceutical composition of a bispecific antibody targeting EGFR and cMet, the bispecific antibody targeting EGFR and cMet comprising a first heavy chain (HC1) comprising a HC1 variable region 1 (VH1); a first light chain (LC1) comprising a light chain variable region 1 (VL1); a second heavy chain (HC2) comprising a HC2 variable region 2 (VH2); and a second light chain (LC2) comprising a light chain variable region 2 (VL2), wherein the VH1 comprises a heavy chain complementarity determining region 1 (HCDR1), a HCDR2 and a HCDR3 comprising amino acid sequences of SEQ ID NOs: 1, 2, and 3, respectively; the VL1 comprises a light chain complementarity determining region 1 (LCDR1), a LCDR2 and a LCDR3 comprising amino acid sequences of SEQ ID NOs: 4, 5 and 6, respectively; the VH2 comprises HCDR1, HCDR2 and HCDR3 amino acid sequences of SEQ ID NOs: 7, 8 and 9, respectively; and the VL2 comprises LCDR1, LCDR2 and LCDR3 amino acid sequences of SEQ ID NOs: 10, 11 and 12, respectively; the method comprising:
   combining a composition comprising about 160 mg/mL of the bispecific antibody, about 30 mM acetate and/or pharmaceutically acceptable acetate salt, about 8.5% sucrose, and about 1 mg/mL L-methionine with polysorbate 80 to a final concentration of about 0.06% (w/v) and EDTA to a final concentration of about 20 μg/mL, optionally rHuPH20 to a final concentration of about 2,000 U/mL, wherein the stable aqueous pharmaceutical composition has about pH 5.7.
35. The method of embodiment 34, wherein the bispecific EGFR-cMet antibody comprises an HC1 variable region comprising the amino acid sequence of SEQ ID NO:13 and a LC1 variable region comprising the amino acid sequence of SEQ ID NO:14.
36. The method of any one of embodiments 34-35, wherein the bispecific EGFR-cMet antibody comprises a HC2 variable region comprising the amino acid sequence of SEQ ID NO:15 and a LC2 variable region comprising the amino acid sequence of SEQ ID NO:16.
37. The method of any one of embodiments 34-36, wherein the antibody comprises a heavy chain 1 (HC1) comprising the amino acid sequence of SEQ ID NO:17 and a light chain 1 (LC1) comprising the amino acid sequence of SEQ ID NO:18.
38. The method of any one of embodiments 34-37, wherein the antibody comprises a HC2 comprising the amino acid sequence of SEQ ID NO:19 and a LC2 comprising the amino acid sequence of SEQ ID NO:20.
39. The method of any one of embodiments 34-38, wherein the antibody is amivantamab or a biosimilar thereof.
40. A kit comprising the stable aqueous pharmaceutical composition of any one of embodiments 1-29 and instructions for use thereof.
41. An article of manufacture comprising a container holding a stable aqueous pharmaceutical composition in accordance with any one of embodiments 1-29.
42. The article of manufacture according to embodiment 41, wherein the container is a vial with a stopper pierceable by a syringe.
43. The article of manufacture according to embodiment 42, wherein the vial is a single-use vial.
44. A pharmaceutical composition of any one of embodiments 1-29 for use in the treatment of cancer.
45. The pharmaceutical composition of embodiment 44, wherein the cancer comprises lung cancer.
46. The pharmaceutical composition of embodiment 44, wherein the cancer comprises non-small cell lung cancer (NSCLC).
47. A pharmaceutical composition of any one of embodiments 1-29 for use in the preparation of a medicament for treating cancer.
48. Use of a pharmaceutical composition for treating cancer in a subject in need thereof by administering the pharmaceutical composition of any one of embodiments 1-29.
49. Use of a pharmaceutical composition according to embodiment 48, wherein the administration is subcutaneous.
50. Use of a pharmaceutical composition according to embodiment 49 for reducing infusion-related reactions in a subject treated with amivantamab.

### EXAMPLES

The following examples are provided to further describe some of the embodiments disclosed herein. The examples are intended to illustrate, not to limit, the disclosed embodiments.

### Description of Analytical Tests Used Herein

### Analytical Tests—General Characterization

### Color of Solution

Color of solution is monitored for drug product (DP) to assess appearance and ensure it is consistent with previous batches at release and over the shelf life. Color of solution may be an indicator of product stability. To determine Color of solution, test samples are visually compared to a defined set of reference solutions.

A defined volume of liquid content is transferred into a pre-scored ampoule of same dimensions as the reference solutions. Then the content of the ampoule is visually compared to European Pharmacopoeia color reference solutions. The degree of color is determined in diffuse daylight, viewed against a white background.

### Color of Solution Material and Methods

Materials and methods are as described in European Pharmacopoeia 2.2.2, Degree of Coloration of Liquids European Pharmacopoeia (Ph. Eur.) 10th Edition monograph number 20202, July 2019. Briefly, test articles are compared against B (Brown), BY (Brownish-Yellow), and Y (Yellow) Color Reference Solution Sets.

Color of Solution Results Consistent with Stability

In one embodiment, stability of DP is defined as having a color of solution of colorless to about BY2 or less, about B2 or less, about Y2 or less after storage for about 12 months or more and at a temperature of about 5° C., after storage for about 12 months or more and at a temperature of about 25° C., and/or after storage for about 2 years or more and at a temperature of about 5° C. In a preferred embodiment, stability of DP is defined as having a color of solution of colorless to about BY4 or less, to about B4 or less, to about Y4 or less after storage for about 12 months or more and at a temperature of about 5° C., after storage for about 12 months or more and at a temperature of about 25° C., and/or after storage for about 2 years or more and at a temperature of about 5° C. In the most preferred embodiment, stability of DP is defined as having a color of solution of colorless to about BY5 or less, to about B5 or less, to about Y5 or less after storage for about 12 months or more and at a temperature of about 5° C., after storage for about 12 months or more and at a temperature of about 25° C., and/or after storage for about 2 years or more and at a temperature of about 5° C.

pH

### pH Materials and Methods

A daily calibrated electronic pH meter with standardized pH electrode is used to measure the pH of test articles. All calibration solutions, reference buffers, and test articles are equilibrated to, and maintained at, 25° C. prior to and during testing.

pH Results Consistent with Stability

In one embodiment, stability of DP is defined as having a pH range of 5.0 to 6.4 after storage for about 12 months or more and at a temperature of about 5° C., after storage for about 12 months or more and at a temperature of about 25° C., and/or after storage for about 2 years or more and at a temperature of about 5° C. In a preferred embodiment, stability of DP is defined as a pH range of 5.2 to 6.2 after storage for about 12 months or more and at a temperature of about 5° C., after storage for about 12 months or more and at a temperature of about 25° C., and/or after storage for about 2 years or more and at a temperature of about 5° C. In the most preferred embodiment, stability of DP is defined as having a pH range of 5.4 to 6.0 after storage for about 12 months or more and at a temperature of about 5° C., after storage for about 12 months or more and at a temperature of about 25° C., and/or after storage for about 2 years or more and at a temperature of about 5° C.

### Turbidity

### Turbidity Materials and Methods

The materials and methods are based on European Pharmacopoeia 2.2.1, Clarity and Degree of Opalescence of Liquids.

Turbidity Results Consistent with Stability

Test results are reported in nephelometric turbidity units (NTU). In one embodiment, stability of DP is defined as having a turbidity value of about 18 NTU or less after storage for about 12 months or more and at a temperature of about 5° C., after storage for about 12 months or more and at a temperature of about 25° C., and/or after storage for about 2 years or more and at a temperature of about 5° C. In a preferred embodiment, stability of DP is defined as having a turbidity value of about 13 NTU or less after storage for about 12 months or more and at a temperature of about 5° C., after storage for about 12 months or more and at a temperature of about 25° C., and/or after storage for about 2 years or more and at a temperature of about 5° C. In the most preferred embodiment, stability of DP is defined as having a turbidity value of about 8 NTU or less after storage for about 12 months or more and at a temperature of about 5° C., after storage for about 12 months or more and at a temperature of about 25° C., and/or after storage for about 2 years or more and at a temperature of about 5° C.

### Analytical Tests—Particulate Matter

Particulate Matter (Sub-visible) Materials and Methods—All materials and methods are compliant with United States Pharmacopeia <788> Particulate Matter. A Compendial compliant Liquid Particle Counter instrument equipped with a compendial volume sampler set-up is used. Test articles are equilibrated to room temperature for at least 60 minutes, but no longer than 10 hours, prior to testing. Test article vials are pooled in manner compliant with United States Pharmacopeia <788> Particulate Matter. As instructed by United States Pharmacopeia <788> Particulate Matter, four portions of pooled test article, each of appropriate volume, are removed and the number of particles equal to or greater than 10 μm and 25 μm are counted per portion. Results obtained for the first portion are disregarded and the remaining three results are used to calculate the mean number of particles for the preparation examined.

Particle Analysis (sub-vis) compendia compliant results—Testing results are to comply with United States Pharmacopoeia <788> Particulate Matter, European Pharmacopoeia 2.9.19, and Japanese Pharmacopoeia XVII/6.07 Particulate Contamination: Sub-visible particles. As such, the average number of particles present in the units tested should not exceed 6000 particles per container for particles size equal to 10 μm or greater and should not exceed 600 particles per container for particles size equal to 25 μm or greater.

### Analytical Tests—Purity

Capillary Electrophoresis Sodium Dodecyl Sulfate (cSDS)-Reduced

cSDS Reduced Materials and Methods—Analysis employs a commercial capillary electrophoresis system with a bare fused silica capillary, 50 μm i.d.×30.2 cm length in a temperature-controlled cartridge; the capillary is equipped with a detection window transparent to ultraviolet light. The capillary is rinsed electrokinetically before each injection. The capillary is loaded with a sieving matrix consisting of an entangled polymer solution before each sample analysis. The method utilizes an SDS-MW gel migration buffer and certified protein molecular weight standards spanning a range of approximately 10 to 148 kDa. The instrument's ultraviolet absorption spectrophotometer detector is set at a wavelength of 220 nm and the capillary temperature is set to 25° C. For reducing sample treatment conditions, the test article (in duplicate) is mixed with SDS and 2-mercaptoethanol and then heated for a defined time and temperature to fully denature and reduce the protein. The reduced sample is injected electro-kinetically by applying a voltage of 5 kV across the capillary for approximately 20 seconds, and then analyzed by application of a greater electric field for approximately 35 minutes. Detection is accomplished by absorbance in the far ultraviolet region of the spectrum, 220 nm. Percent of total signal data is collected for the light chain, heavy chain, and aglycosylated heavy chain (AG HC).

cSDS Reduced results consistent with stability. In one embodiment, stability of DP is defined as having a percent purity ≥88.0%, AG HC≤11.0%, and no new peak >1.5% compared to a validated stock of amivantamab Reference Material after storage for about 12 months or more and at a temperature of about 5° C., after storage for about 12 months or more and at a temperature of about 25° C., and/or after storage for about 2 years or more and at a temperature of about 5° C. In a preferred embodiment, stability of DP is defined a percent purity about or more than 91.0%, AG HC less than or about 8.0%, and no new peak more than 1.0% compared to Reference Material after storage for about 12 months or more and at a temperature of about 5° C., after storage for about 12 months or more and at a temperature of about 25° C., and/or after storage for about 2 years or more and at a temperature of about 5° C. In the most preferred embodiment, stability of DP is defined as having a percent purity about or more than 94.0%, AG HC less than or about 5.0%, and no new peak more than 1.0% compared to Reference Material after storage for about 12 months or more and at a temperature of about 5° C., after storage for about 12 months or more and at a temperature of about 25° C., and/or after storage for about 2 years or more and at a temperature of about 5° C.

Capillary Electrophoresis Sodium Dodecyl Sulfate (cSDS)-Non-Reduced

cSDS Non-reduced Materials and Methods—Analysis employs a commercial capillary electrophoresis system with a bare fused silica capillary, 50 μm i.d.×30.2 cm length in a temperature-controlled cartridge; the capillary is equipped with a detection window transparent to ultraviolet light. The capillary is rinsed electrokinetically before each injection. The capillary is loaded with a sieving matrix consisting of an entangled polymer solution before each sample analysis. The method utilizes an SDS-MW gel migration buffer, certified protein molecular weight standards spanning a range of approximately 10 to 148 kDa, and a validated amivantamab Reference Material sample. The instrument's ultraviolet absorption spectrophotometer detector is set at a wavelength of 220 nm and the capillary temperature is set to 25° C. For non-reduced sample treatment conditions, the test article (in duplicate) is mixed with SDS and the alkylating reagent (N-Ethylmaleimide, to prevent disulfide bond shuffling or reformation). It is then heated for a defined time and temperature to fully denature the protein and minimize formation of fragments and artefact bands. The non-reduced sample is injected electrokinetically by applying a voltage of 5 kV across the capillary for approximately 20 seconds, and then analyzed by application of a greater electric field for approximately 35 minutes. Detection is accomplished by absorbance in the far ultraviolet region of the spectrum, 220 nm. Percent of total signal data is collected. The data is also analyzed for the presence of new peaks versus amivantamab Reference Material. Percent purity is defined as percent heavy chain+percent light chain.

cSDS Non-Reduced results consistent with stability. In one embodiment, stability of DP is defined as having a percent purity of about 88.0% or more and no new peak more than 1.5% compared to Reference Material after storage for about 12 months or more and at a temperature of about 5° C., after storage for about 12 months or more and at a temperature of about 25° C., and/or after storage for about 2 years or more and at a temperature of about 5° C. In a preferred embodiment, stability of DP is defined as percent purity of about 90.0% or more and no new peak more than 1.0% compared to Reference Material after storage for about 12 months or more and at a temperature of about 5° C., after storage for about 12 months or more and at a temperature of about 25° C., and/or after storage for about 2 years or more and at a temperature of about 5° C. In the most preferred embodiment, stability of DP is defined as having a percent purity of about 94.0% or more and no new peak more than 1.0% compared to Reference Material after storage for about 12 months or more and at a temperature of about 5° C., after storage for about 12 months or more and at a temperature of about 25° C., and/or after storage for about 2 years or more and at a temperature of about 5° C.

### Size Exclusion High Performance Liquid Chromatography (SE-HPLC)

SE-HPLC Materials and Methods—Reference Material and test articles are diluted to a target protein concentration. A 20 μl volume of analyte is injected onto a 7.8 mm×30 cm size exclusion column with 5 μm particle size silica base, with a fractionation range of 10 to 500 kDa. Aqueous phosphate buffer is used as the mobile phase at a flow rate of 0.7 mL/minute and the absorbance of the eluate is monitored continuously at 280 nm. Monomer (main component or main peak), aggregates (high molecular weight species, or HMWS), and fragments (low molecular weight species, or LMWS) are separated on the column and elute at different retention times. The amounts of these species are measured by monitoring peak absorbance at 280 nm.

SE-HPLC Results Consistent with Stability

Main Component—In one embodiment, stability of DP is defined as having a Main Component about 90.0% or more after storage for about 12 months or more and at a temperature of about 5° C., after storage for about 12 months or more and at a temperature of about 25° C., and/or after storage for about 2 years or more and at a temperature of about 5° C. In a preferred embodiment, stability of DP is defined as having a Main Component about 95.0% or more after storage for about 12 months or more and at a temperature of about 5° C., after storage for about 12 months or more and at a temperature of about 25° C., and/or after storage for about 2 years or more and at a temperature of about 5° C. In the most preferred embodiment, stability of DP is defined as having a Main Component about 97.0% after storage for about 12 months or more and at a temperature of about 5° C., after storage for about 12 months or more and at a temperature of about 25° C., and/or after storage for about 2 years or more and at a temperature of about 5° C. High Molecular Weight Species (HMWS)—In one embodiment, stability of DP is defined as having a HMWS of about 10.0% or less after storage for about 12 months or more and at a temperature of about 5° C., after storage for about 12 months or more and at a temperature of about 25° C., and/or after storage for about 2 years or more and at a temperature of about 5° C. In a preferred embodiment, stability of DP is defined as having a HMWS of about 5.0% or less after storage for about 12 months or more and at a temperature of about 5° C., after storage for about 12 months or more and at a temperature of about 25° C., and/or after storage for about 2 years or more and at a temperature of about 5° C. In the most preferred embodiment, stability of DP is defined as having a HMWS of about 3.0% or less after storage for about 12 months or more and at a temperature of about 5° C., after storage for about 12 months or more and at a temperature of about 25° C., and/or after storage for about 2 years or more and at a temperature of about 5° C. Low Molecular Weight Species (LMWS)—In one embodiment, stability of DP is defined as having a LMWS about 5.0% or less after storage for about 12 months or more and at a temperature of about 5° C., after storage for about 12 months or more and at a temperature of about 25° C., and/or after storage for about 2 years or more and at a temperature of about 5° C. In a preferred embodiment, stability of DP is defined as having a LMWS of about 2.0% or less after storage for about 12 months or more and at a temperature of about 5° C., after storage for about 12 months or more and at a temperature of about 25° C., and/or after storage for about 2 years or more and at a temperature of about 5° C. In the most preferred embodiment, stability of DP is defined as having a LMWS about 1.0% or less after storage for about 12 months or more and at a temperature of about 5° C., after storage for about 12 months or more and at a temperature of about 25° C., and/or after storage for about 2 years or more and at a temperature of about 5° C.

Capillary Isoelectric Focusing (cIEF)

cIEF Materials and Methods—The analytical procedure is performed on a commercially available imaging cIEF analyzer equipped with an auto sampler. Analysis employs a 100-μm inner wall-coated silica capillary with an outer wall polyimide coating. In addition, an analyte solution of dilute phosphoric acid and methylcellulose, a catholyte solution of sodium hydroxide and methylcellulose, and defined type and amount of ampholytes are used. The test articles are treated with carboxypeptidase B (CPB) to remove C-terminal lysine and eliminate ambiguities introduced by the presence of multiple C-terminal variants for each charged species. The instrument's autosampler is set to 4° C. for both pre-focusing and focusing. The Pre-focusing voltage and time are 1500 V and 1 minute respectively. The Focusing voltage and time are 3000 V and 7 minutes respectively.

cIEF Results Consistent with Stability

Main Peak—In one embodiment, stability of DP is defined as having a Main Peak of 37-87% after storage for about 12 months or more and at a temperature of about 5° C., after storage for about 12 months or more and at a temperature of about 25° C., and/or after storage for about 2 years or more and at a temperature of about 5° C. In a preferred embodiment, stability of DP is defined as having a Main Peak of 47-87% after storage for about 12 months or more and at a temperature of about 5° C., after storage for about 12 months or more and at a temperature of about 25° C., and/or after storage for about 2 years or more and at a temperature of about 5° C. In the most preferred embodiment, stability of DP is defined as having a Main Peak of 57-87% after storage for about 12 months or more and at a temperature of about 5° C., after storage for about 12 months or more and at a temperature of about 25° C., and/or after storage for about 2 years or more and at a temperature of about 5° C.

Sum of acidic peaks—In one embodiment, stability of DP is defined as having a Sum of acidic peaks totaling 10-60% after storage for about 12 months or more and at a temperature of about 5° C., after storage for about 12 months or more and at a temperature of about 25° C., and/or after storage for about 2 years or more and at a temperature of about 5° C. In a preferred embodiment, stability of DP is defined as having a Sum of acidic peaks totaling 10-50% after storage for about 12 months or more and at a temperature of about 5° C., after storage for about 12 months or more and at a temperature of about 25° C., and/or after storage for about 2 years or more and at a temperature of about 5° C. In the most preferred embodiment, stability of DP is defined as having a Sum of acidic peaks totaling 10-40% after storage for about 12 months or more and at a temperature of about 5° C., after storage for about 12 months or more and at a temperature of about 25° C., and/or after storage for about 2 years or more and at a temperature of about 5° C.

Sum of basic peaks—In one embodiment, stability of DP is defined as having a Sum of basic peaks totaling about 12.0% or less after storage for about 12 months or more and at a temperature of about 5° C., after storage for about 12 months or more and at a temperature of about 25° C., and/or after storage for about 2 years or more and at a temperature of about 5° C. In a preferred embodiment, stability of DP is defined as having a Sum of basic peaks totaling about 10.0% or less after storage for about 12 months or more and at a temperature of about 5° C., after storage for about 12 months or more and at a temperature of about 25° C., and/or after storage for about 2 years or more and at a temperature of about 5° C. In the most preferred embodiment, stability of DP is defined as having a Sum of basic peaks totaling about 8.0% or less after storage for about 12 months or more and at a temperature of about 5° C., after storage for about 12 months or more and at a temperature of about 25° C., and/or after storage for about 2 years or more and at a temperature of about 5° C.

### Analytical Tests—Quantity

### Protein Concentration by A280

Protein concentration of the drug product is determined by quantification of the absorbance at 280 nm (A280).

### Protein Concentration by A280 Materials and Methods

Measurement of protein concentration is performed using a qualified and calibrated double beam UV-Vis spectrophotometer. Test articles are diluted 1:125 using 0.9% (w/v) NaCl. Samples are measured using quartz semi-micro cuvettes (1.4 mL) with a 1 cm path length and black or frosted sides. The Spectrophotometer is set to a Wavelength of 280 nm, a slit width of 1 nm, and a response of one (1) second. 0.9% (w/v) NaCl is used as the Blank control. Protein concentration (mg/mL) is calculated by dividing the product of the Test article absorbance and dilution factor by the product of the antibody's Absorptivity Constant and instrument's path length (for example, but not limited to an amivantamab's Absorptivity Constant of 1.40 (mg/mL)⁻¹ cm⁻¹ and instrument's path length of 1 cm).

Protein Concentration Results Consistent with Stability of DP

In one embodiment, stability of DP is defined as having a protein concentration of 128 to 192 mg/mL after storage for about 12 months or more and at a temperature of about 5° C., after storage for about 12 months or more and at a temperature of about 25° C., and/or after storage for about 2 years or more and at a temperature of about 5° C. In a preferred embodiment, stability of DP is defined as having a protein concentration of 144 to 176 mg/mL after storage for about 12 months or more and at a temperature of about 5° C., after storage for about 12 months or more and at a temperature of about 25° C., and/or after storage for about 2 years or more and at a temperature of about 5° C. In the most preferred embodiment, stability of DP is defined as having a protein concentration of 150 mg/mL to 170 mg/mL after storage for about 12 months or more and at a temperature of about 5° C., after storage for about 12 months or more and at a temperature of about 25° C., and/or after storage for about 2 years or more and at a temperature of about 5° C.

### Analytical Tests—Potency

### Potency (Epidermal Growth Factor Receptor (EGFR) Binding)

The in vitro binding of drug product to EGFR is demonstrated using a homogeneous competitive time resolved fluorescence resonance energy transfer (TR-FRET) assay format. In this procedure, varying concentrations of unlabeled bispecific EGFR-cMet antibody sample compete with donor fluorophore (Europium (Eu) chelate) labeled bispecific EGFR-cMet antibody for binding to an acceptor fluorophore (Cy5)-labeled EGFR antigen. Excitation of the donor fluorophore results in a transfer of energy to the bound acceptor fluorophore (FRET process). The resultant FRET is detected by emission of light at 665 nm using a microplate reader capable of measuring time-resolved fluorescence. Sample dose response curves are compared to the RM.

EGFR Binding Materials and Methods. Certified commercial EGFR, a recombinant human EGFR/ErbB1/HER1 with C-terminal His tag is reacted with certified commercial Cy5 Mono NHS Ester to produce Cy5-labeled EGFR. Validated bispecific EGFR-cMet antibody is reacted with certified commercial Europium (Eu) chelate to produce Eu-labeled bispecific EGFR-cMet antibody. Serial dilutions of bispecific EGFR-cMet antibody Reference Material (RM), assay control and test articles are tested in parallel on the same assay plate. Eu labeled bispecific EGFR-cMet antibody is added to each RM, assay control, and test article followed gentle shaking of the assay plate. Cy5-EGFR is then similarly added, the assay plate again gentle shaken, and incubated in the dark for 4±1 hours. Fluorescence is then measured by spectrophotometry at 665 nm, plotted against antibody concentration and analyzed by a four-parameter logistic model. The antibody concentration required to obtain half of the maximum fluorescence response (EC50) is determined for RM, assay control and samples. The potencies of assay control and samples are calculated based on the ratio of the sample (or control) and RM EC50 values and reported as a percentage activity relative to the RM.

EGFR Binding Activity results consistent with stability. In one embodiment, stability of DP is defined as 50%-150% binding activity relative to Reference Material after storage for about 12 months or more and at a temperature of about 5° C., after storage for about 12 months or more and at a temperature of about 25° C., and/or after storage for about 2 years or more and at a temperature of about 5° C. In a preferred embodiment, stability of DP is defined as 60%-140% binding activity relative to Reference Material after storage for about 12 months or more and at a temperature of about 5° C., after storage for about 12 months or more and at a temperature of about 25° C., and/or after storage for about 2 years or more and at a temperature of about 5° C. In the most preferred embodiment, stability of DP is defined as ranging between about 80% to 120% binding activity relative to Reference Material after storage for about 12 months or more and at a temperature of about 5° C., after storage for about 12 months or more and at a temperature of about 25° C., and/or after storage for about 2 years or more and at a temperature of about 5° C.

Potency (cMet Binding)

The in vitro binding of bispecific EGFR-cMet antibody to c-MET is demonstrated using a homogeneous competitive time resolved fluorescence resonance energy transfer (TR-FRET) assay format. In this procedure, varying concentrations of unlabeled bispecific EGFR-cMet antibody sample compete with donor fluorophore (Europium (Eu) chelate) labeled bispecific EGFR-cMet antibody for binding to an acceptor fluorophore (Cy5)-labeled c-MET antigen. Excitation of the donor fluorophore results in a transfer of energy to the bound acceptor fluorophore (FRET process). The resultant FRET is detected by emission of light at 665 nm using a microplate reader capable of measuring time-resolved fluorescence. Sample dose response curves are compared to the Reference Material (RM).

c-MET Binding Materials and Methods. Certified commercial cMet, a recombinant cMet/HGFR with c-terminal His-tag is reacted with certified commercial Cy5 Mono NHS Ester to produce Cy5-labeled c-MET. Validated bispecific EGFR-cMet antibody is reacted with certified commercial Europium (Eu) chelate to produce Eu labeled bispecific EGFR-cMet antibody. Serial dilutions of bispecific EGFR-cMet antibody RM, assay control and test articles are tested in parallel on the same assay plate. Eu labeled bispecific EGFR-cMet antibody is added to each RM, assay control, and test article followed by gentle shaking of the assay plate. Cy5-c-MET is then similarly added, the assay plate again gently shaken, and incubated in the dark for 4±1 hours. Fluorescence is then measured by spectrophotometry at 665 nm, plotted against antibody concentration and analyzed by a four-parameter logistic model. The antibody concentration required to obtain half of the maximum fluorescence response (EC50) is determined for RM, assay control and samples. The potencies of assay control and samples are calculated based on the ratio of the sample (or control) and RM EC50 values and reported as a percentage activity relative to the RM.

cMet Binding Activity results consistent with stability. In one embodiment, stability of DP is defined as ranging between about 50% to about 150% binding activity relative to Reference Material after storage for about 12 months or more and at a temperature of about 5° C., after storage for about 12 months or more and at a temperature of about 25° C., and/or after storage for about 2 years or more and at a temperature of about 5° C. In a preferred embodiment, stability of DP is defined as ranging between about 60% to 140% binding activity relative to Reference Material after storage for about 12 months or more and at a temperature of about 5° C., after storage for about 12 months or more and at a temperature of about 25° C., and/or after storage for about 2 years or more and at a temperature of about 5° C. In the most preferred embodiment, stability of DP is defined as ranging about 80% to about 120% binding activity relative to Reference Material after storage for about 12 months or more and at a temperature of about 5° C., after storage for about 12 months or more and at a temperature of about 25° C., and/or after storage for about 2 years or more and at a temperature of about 5° C.

Potency rHuPH20 Activity

In-vitro rHuPH20 hyaluronidase enzymatic activity is determined by measuring turbidity when hyaluronic acid (HA), a substrate of rHuPH20, binds with acidified serum. The determination of hyaluronidase activity is based on the formation of a precipitate when hyaluronic acid (HA) binds with acidified serum. The activity is measured by incubating hyaluronidase with HA for 30 minutes in a 96-well plate format at 37° C. and then precipitating the undigested HA with the addition of acidified serum. The resulting turbidity is measured at 640 nm and the decrease in turbidity resulting from enzymatic cleavage of the HA substrate is a measure of the hyaluronidase activity.

rHuPH20 Activity Materials and Methods

The assay method is based on the United Stated Pharmacopeia Monograph USP29-NF24 Hyaluronidase for Injection. Standard reagents include 500 mM acetate buffer (pH 3.1), 100 mM acetate buffer (pH 3.1), Sterile Water for Irrigation (SWFI), Human Serum Albumin (HSA) 25% (NDC #68209-643-02), Horse Serum, 50 mg/mL sodium hyaluronate, and rHuPH20. Assay specific reagents are listed in table 1 below. The enzyme diluent, Horse Serum Working solution (2.8%), and 0.7 mg/mL HA Substrate are prepared on the day of the assay. The HA substrate tube is stored at 2-8° C. until ready for use.

TABLE 1
Assay Specific Reagents

| Reagents | Compositions |
|---|---|
| Reaction buffer | 50 mM MES¹, 140 mM NaCl, pH 5.5 |
| Serum Stock Solution | 33% horse serum in 100 mM acetate buffer |
| Enzyme Diluent | l mg/mL HAS, 25 mM Reaction Buffer |
| Horse Serum Working Solution | 2.8% horse serum in 100 mH acetate buffer |
| Sodium Hyaluronate (HA) Substrate | 0.7 mg/mL sodium hyaluronate in 25 mM |
| Solution | Reaction Buffer |
| rHuPH20 Working Reference Standard | 81.2 U/mL rHuPH20 |
| (WRS) | 9.3 U/mL rHuPH20 |
| rHuPH20 Control (Check Standard) | |

¹2-(N-Morpholino)ethanesulfonic acid hydrate, 4-Morpholineethanesulfonic acid

### Assay and Sample Preparation

Prior to start of assay, an empty 96-well Reaction Plate is placed in an Eppendorf Thermo-mixer and set to 15° C. and allowed to equilibrate for a minimum of 30 minutes. Also, a heat block is placed in a 37° C. incubator and allowed to equilibrate at the appropriate temperature for at least 2 hours prior to starting the enzyme reaction.

Test samples with an expected rHuPH20 activity of 2,000 U/mL are diluted with Enzyme Diluent to 9.3 U/mL. Aliquots of rHuPH20 controls and diluted test samples and are loaded into a 96-well Transfer Plate. In separate Dilution Plate, designated volumes of Enzyme Diluent are aliquoted into designated wells. WRS is aliquoted in triplicate into designated wells and then serially diluted in designated wells containing enzyme diluent. The data from these wells will be used to generate a six-point calibration curve. Fixed volumes of rHuPH20 controls and diluted samples are transferred in duplicate from the transfer plate into designated wells in the Dilution Plate.

### Enzyme Reaction

The HA substrate solution is removed from 2-8° C. storage and gently mixed by 3-4 inversions. While maintaining the 15° C. equilibrated 96-well Reaction Plate in the Thermo-mixer, a fixed volume of HA substrate solution is aliquoted into the corresponding well locations of the Dilution Plate. The standards, controls and samples from the Dilution Plate are then transferred to their corresponding well locations of the Dilution Plate. The 15° C. Thermo-mixer is then used to mix the Reaction Plate at 900 rpm for 10 seconds. The Reaction Plate is removed from the Thermo-mixer, a plate lid placed on the reaction plate, and immediately transferred into the pre-incubated heat block inside the 37° C. incubator. The Reaction Plate is incubated for 30±5 minutes at 37° C.

### Stop Reaction and Development

Once the 37° C. incubation is complete, remove the covered Reaction Plate from the incubator and immediately press the covered plate into freshly dispensed ice in an ice bucket and start timer for 2 minutes. Leave plate untouched during the 2-minute incubation. After 2 minutes, wipe the bottom of the plate (e.g. Kimwipes) to remove any condensation or water and transfer the reaction plate to the 15° C. Thermo-mixer. Place the Thermo-mixer lid on and incubate at 15° C. for 10 minutes.

After 10 minutes, immediately remove the Thermo-mixer lid and, a fixed volume of horse serum working solution is aliquoted to each well. The plate is then covered with the Thermo-mixer lid and start a timer for 20±5 minutes at 15° C. After the 20±5 minutes incubation, the reaction plate is transferred from the 15° C. Thermo-mixer to a 96-well Plate reader. The optical density of the samples is then measured at 640 nm.

### Data Analysis

The known rHuPH20 Activity (U/mL) values of the serially diluted WRS samples are plotted against their corresponding measured optical density (OD) values and a curve fit equation is obtained. The rHuPH20 activities of each sample dilution in the wells of the 96-well plate are calculated by using the individual OD values and the curve fit equation obtained from the Reference Material curve.

rHuPH20 activity results consistent with stability of DP. In one embodiment, stability of DP is defined as having rHuPH20 activity of 1000 U/mL to 3000 U/mL after storage for about 12 months or more and at a temperature of about 5° C., after storage for about 12 months or more and at a temperature of about 25° C., and/or after storage for about 2 years or more and at a temperature of about 5° C. In a preferred embodiment, stability of DP is defined as having rHuPH20 activity of 1500 U/mL to 2500 U/mL after storage for about 12 months or more and at a temperature of about 5° C., after storage for about 12 months or more and at a temperature of about 25° C., and/or after storage for about 2 years or more and at a temperature of about 5° C. In the most preferred embodiment, stability of DP is defined as having rHuPH20 activity of 1800 U/mL to 2200 U/mL after storage for about 12 months or more and at a temperature of about 5° C., after storage for about 12 months or more and at a temperature of about 25° C., and/or after storage for about 2 years or more and at a temperature of about 5° C.

### Analytical Tests—Surfactant

### Polysorbate-80 Quantification

Polysorbate 80 is quantitatively determined by mixed-mode ion-exchange/hydrophobic HPLC.

PS 80 Materials and Methods. Analysis conducted with a gradient HPLC equipped with a 2.1×20 mm on-line column containing a 30 μm water-wetable, mixed-mode polymeric spherical sorbent particles, an ELSD, and a temperature-controlled column compartment at 30° C. The flow rate is set to 1 mL/minute and the ELSD evaporator temperature is set to 50° C. Mobile Phase A is 2% v/v Formic acid in water and Mobile Phase B is 2% v/v Formic acid in Isopropyl alcohol. Neat polysorbate 80 is used to create calibration and check standards. Test article samples are injected neat.

Polysorbate 80 results consistent with stability of DP. In one embodiment, stability of DP is defined as a PS80 concentration of 0.03-0.08% after storage for about 12 months or more and at a temperature of about 5° C., after storage for about 12 months or more and at a temperature of about 25° C., and/or after storage for about 2 years or more and at a temperature of about 5° C. In a preferred embodiment, stability of DP is defined as a PS 80 concentration of 0.04-0.08% after storage for about 12 months or more and at a temperature of about 5° C., after storage for about 12 months or more and at a temperature of about 25° C., and/or after storage for about 2 years or more and at a temperature of about 5° C. In the most preferred embodiment, stability of DP is defined as a PS 80 concentration of 0.05-0.08% after storage for about 12 months or more and at a temperature of about 5° C., after storage for about 12 months or more and at a temperature of about 25° C., and/or after storage for about 2 years or more and at a temperature of about 5° C.

### Analytical Tests—Routine Characterization

### Peptide Map

The purpose of this test is to measure the levels of post-translational modifications, such as oxidation, deamidation, and isomerization, that may be present in the antibody structure. Test articles are enzymatically digested to yield peptide segments. These peptides are then evaluated by Ultra High-Performance Liquid Chromatography Mass Spectroscopy (UPLC-MS). Each analyzed peptide sequence is identified relative to its known location within the overall antibody structure. Post-translational modifications are determined by comparing the measured mass of the identified peptide sequence with its expected mass.

Peptide Mapping materials and methods. Samples are denatured with 6 M Guanidine, 50 mM Tris pH 8.0, 5 mM EDTA and filtered using 30 kDa centrifugal filter device (flow through discarded). The denatured samples are reduced with 1 M Dithiothreitol (DTT), followed by alkylation with 1 M sodium Iodoacetate, and further treated with DTT to quench the reaction. The reaction mixture is exchanged into digestion buffer (50 mM Tris pH 7.0, with 1 mM CaCl₂) via Sephadex G-25 columns with separate columns used for blanks, Reference Material, and test articles. An aliquot of 1 mg/mL Trypsin stock solution is added to the sample in digestion buffer yielding a 20 μL/mL trypsin concentration. The solution is incubated at 37° C. for 2 hours±30 minutes. The trypsinized solution is allowed to cool to room temperature and the enzyme is inactivated with Trifluoroacetic acid. The treated samples are evaluated by Ultra High-Performance Liquid Chromatography Mass Spectroscopy (UPLC-MS) equipped with a Waters Acquity BEH (Ethylene Bridged Hybrid) C18, 2.1×100 mm, 1.7 μm, 130A column and an attached auto sampler. Mobile phase A is 0.1% Formic Acid in water Mobile phase B is, 0.1% FA in acetonitrile (mobile phase B). The autosampler is set to 2-8° C., the column is set to 40° C. and the flow rate is set to 500 μL/minute. Eluted peptides were subject to electrospray ionization and detected using a calibrated on-line mass spectrometry.

### Example 1: Formulation Screening Studies

Two formulation screening studies were conducted to evaluate stability trends for high concentration (175 mg/mL) amivantamab formulated with varied pH values, formulation buffer species, and buffer concentrations.

### Study 1

The first study evaluated test formulations consisting of a range of pH values and corresponding buffer species with all other formulation components held at a fixed value. (Table 2).

TABLE 2
Composition of Study 1 Test Formulations

| Test | Buffer | | API | Buffer Conc. | Sucrose | EDTA | Polysorbate 80 |
|---|---|---|---|---|---|---|---|
| Formulation | Species | pH | (mg/mL) | (mM) | % (w/v) | (ug/mL) | % (w/v) |
| 1 | Acetate | 5.1 | 175 | 10 | 7.5 | 20 | 0.06 |
| 2 | Histidine | 5.6 | | | | | |
| 3 | Histidine | 6.4 | | | | | |
| 4 | Phosphate | 7.2 | | | | | |

The test formulations were held under normal (5° C.) and stress (40° C.) stability conditions for three weeks. Test formulations were then assayed by SEC to evaluate the percent of aggregate, monomer, and fragment observed per test formulation.

As shown in Table 3 below, increased pH values generally correlated with increase % aggregate at both Normal (5° C.) and Stressed (40° C.) stability conditions. As expected, the aggregation values were greater under stressed conditions. Overall, Formulations 3 and 4 underperformed versus Formulations 1 and 2. Therefore, high pH formulations and phosphate buffers were not considered for further development

Under normal conditions, Formulations 1 and 2 both exhibited similar results consistent with a stable formulation with Formulation 1 showing slightly less aggregation than Formulation 2. However, under stressed conditions Formulation 2 exhibited the least aggregation, albeit with increased fragment. Based on a seemingly equivalent trade-off between aggregates and fragments, both acetate and histidine buffers at low pH values were considered for further evaluation.

TABLE 3
Test Formulations held at Normal (5° C.) and
Stressed (40° C.) stability conditions

| | | SEC | | |
|---|---|---|---|---|
| Stability | Test | Aggregate | Monomer | Fragment |
| Condition | Formulation | (%) | (%) | (%) |
| Normal | 1 | 0.86 | 98.85 | 0.28 |
| (5° C.) | 2 | 1.13 | 98.6 | 0.27 |
| | 3 | 1.39 | 98.35 | 0.27 |
| | 4 | 2.25 | 97.44 | 0.31 |
| Stressed | 1 | 1.78 | 97.75 | 0.47 |
| (40° C.) | 2 | 1.3 | 96.99 | 1.71 |
| | 3 | 2.42 | 96.79 | 0.51 |
| | 4 | 3.31 | 95.83 | 0.87 |

### Study 2

The second study evaluated buffer concentration. Histidine buffer of pH 5.6 was selected as a representative low pH formulation. All other formulation components held at a fixed value (Table 4).

TABLE 4
Composition of Study 2 Test Formulations

| | Buffer | | | | |
|---|---|---|---|---|---|
| Test | Conc. | Buffer | | API | Sucrose |
| Formulation | (mM) | Species | pH | (mg/mL) | % (w/v) |
| 1 | 10 | Histidine | 5.6 | 175 | 7.5 |

| | |
|---|---|
| 2 | 25 |
| 3 | 50 |

The test formulations were held under stress (40° C.) stability conditions for three weeks. Test formulations were then assayed by SEC to evaluate the percent of aggregate, monomer, and fragment observed per test formulation.

As shown in Table 5 below, increased buffer concentration correlated with decreased % aggregate. Therefore, formulations with a range of buffer concentrations would be considered for further evaluated.

TABLE 5
Test Formulations held at Stressed (40° C.)
stability conditions

| | SEC | | |
|---|---|---|---|
| Test | Aggregate | Monomer | Fragment |
| Formulation | (%) | (%) | (%) |
| 1 | 4.22 | 94.61 | 1.17 |
| 2 | 2.16 | 97.1 | 0.75 |
| 3 | 1.78 | 97.53 | 0.7 |

### Example 2: High Concentration Stability Study

A stability study was conducted to evaluate three high protein concentration formulations with varied buffer concentrations and species (see Table 6 below) The test formulations were held at recommended (5° C.), accelerated (25° C.), and stressed (40° C.) conditions up to 6-months.

TABLE 6
Study Test Formulations

| Formulation | |
|---|---|
| No. | Formulation Composition |
| 1 | 160 mg/mL amivantamab in 10 mM Histidine, |
| | 8.5% sucrose, 1 mg/mL Methionine, |
| | 20 μg/mL EDTA, 0.06% PS-80, pH 5.6 |
| 2 | 160 mg/mL amivantamab in 50 mM Histidine, |
| | 8.5% sucrose, 1 mg/mL Methionine, |
| | 20 μg/mL EDTA, 0.06% PS-80, pH 5.6 |
| 3 | 160 mg/mL amivantamab in 10 mM acetate, |
| | 8.5% sucrose, 1 mg/mL Methionine, |
| | 20 μg/mL EDTA, 0.06% PS-80, pH 5.1 |

The three test formulations were prepared from a 50 mg/mL stock formulation of amivantamab. The stock formulation was concentrated by Tangential Flow Filtration (TFF) and the buffers exchanged by Ultrafiltration/Diafiltration (UF/DF). Test formulations were aliquoted into 30R vials at a fill volume of 15.6 mL. The vials were stoppered, capped, and crimp sealed. The vials were placed on stability at recommended (5° C.), accelerated (25° C.), and stressed (40° C.) conditions. At designated time points, samples are pulled and assayed.

### Study Results

The stability results for test formulations held under recommended, accelerated, and stressed conditions are listed in Tables 7, 8, and 9 below. It was noted at the start (T=0) and throughout the study that the measured pH value for Formulation 3 did not align with its target value. A sample of Formulation 3 was assayed for acetate concentration and was reported to be 30 mM. The shifts in pH and acetate concentration were a result of the Gibbs-Donnan effect, the magnitude of which was affected by the high protein concentration, the pI of the protein, and the pH of the diafiltration buffer used. Therefore, the actual composition of Formulation 3 was 160 mg/mL amivantamab in 30 mM acetate, 8.5% sucrose, 1 mg/mL Methionine, 20 g/mL EDTA, 0.06% PS-80, pH 5.7.

### Stability at 5° C.

All three formulations showed little change in attribute values over time. Any changes observed were slight and consistent with protein degradation over time at 5° C. were of similar magnitude across all formulations.

### Stability at 25° C.

All three formulations showed slight to minimal changes in attribute values over time consistent with protein degradation over time at 25° C. with changes observed of similar magnitude across all formulations except for cIEF attribute values. All formulations showed a steady increase over time in % Sum of Acidic Peaks and corresponding decrease in % Main Peak. While the magnitude of the changes over time were similar for Formulations 2 and 3, the magnitude was markedly greater for Formulation 1.

### Stability at 40° C.

All three formulations showed noticeable changes in attribute values over time consistent with protein degradation over time at 40° C. for up to six months. However, for some attributes, the magnitude of the changes was greater in Formulation 1 than Formulation 2 or 3.

cIEF data for all formulations showed a steady increase in % Sum of Acidic Peaks and corresponding decrease in % Main Peak. However, the magnitude of the changes over time were markedly greater for Formulation 1 than Formulation 2 or 3, particularly between T=0 and three month and between three and six months.

This trend was also seen for SEC with all formulations showing a steady increase in HMWS % Peaks and corresponding decrease in % Main Component. But similar to the cIEF data, the magnitude of increase was similar for Formulations 2 and 3 and markedly greater for Formulation 1.

Color scoring for Formulations 2 and 3 showed noticeable, albeit modest increases in B, BY, and Y color scoring over time. By contrast, Formulation 1 showed a rapid increase in color scoring over time with the 6-month sample receiving the maximum B, BY, and Y color scores.

No changes in pH were observed for Formulations 2 and 3 over six months. However, Formulation 1 showed a marked decrease in pH between three and six months.

Post Translation Modification after Holding for Three Months at 5° C. 25° C., and 40° C.

Post translational modification at T=0 and 3M at 5° C., 25° C., and 40° C. is shown in tables 10-11. At 5° C. and 25° C., all three formulations showed little or slight to minimal changes in attribute values verse T=0 respectively. Within a given temperature, the magnitude of the changes was similar across all formulations and consistent with protein degradation over time at those temperatures.

At 40° C., a marked increase in deamidation at anti-EGFR HC Asn 333/anti-c-Met HC Asn 327 and anti-c-Met HC Asn 55, 59 versus T=0 was observed. A similar trend in isomerization of anti-EGFR HC Asp 99 was observed. The magnitude of the increase deamidation and isomerization were similar across all three formulations.

Also, at 40° C., Formulations 2 and 3 showed noticeable changes in oxidation values versus T=0 consistent with protein degradation over time at 40° C. However, Formulation 1 showed a marked increase in the magnitude of the oxidation verse Formulations 2 and 3 as well as its corresponding T=0 values for anti-EGFR HC Met 103, anti-EGFR HC Met 108, anti-EGFR HC Met 260/anti-c-Met HC Met 254, anti-EGFR HC Met 436/anti-c-Met HC Met 430, and anti-c-Met LC Trp 32, Trp 35.

### Discussion and Conclusion

Throughout the study Formulation 1 showed decreased stability attributes versus Formulations 2 and 3. The change in pH at accelerated temperatures suggested that Formulation 1 (10 mM Histidine) had a poor buffer capacity for amivantamab at high (160 mg/mL) concentration.

Formulations 2 and 3 showed similar stability profiles over the course of the study. However, at all study time points and temperatures, Formulation 2 appeared as a slightly opalescent liquid whereas Formulation 3 appeared as a clear liquid. Similarly, Formulation 3 showed lower turbidity values than Formulation 2 throughout the study.

TABLE 7
Stability Study 5° C.

| | | | | | | Particulate Matter | | | SE-HPLC | | |
|---|---|---|---|---|---|---|---|---|---|---|---|
| | | | | | | (Sub-visible) | | | | | Main |
| Time- | Formu- | | Turbidity | | | ≥10 μm | ≥25 μm | A280 | LMWS | HMWS | Component |
| point | lation | pH | NTU | Color | Apperance | per mL | per mL | mg/mL | % | % | % |
| T0 | 1 | 6.1 | 5.9 | ≤B9, ≤BY7, ≤Y7 | clear liquid | 3 | 0 | 163.5 | 0.00 | 1.20 | 98.80 |
| T3 m | | 6 | 6.3 | ≤B5; ≤BY4; ≤Y4 | slightly | 1 | 0 | 162.9 | 0.05 | 1.48 | 98.50 |
| | | | | | opalescent | | | | | | |
| | | | | | liquid | | | | | | |
| T6 m | | 6 | 5.85 | ≤B9, ≤BY7, ≤Y7 | slightly | 3 | 1 | 162.0 | 0.08 | 1.52 | 98.40 |
| | | | | | opalescent | | | | | | |
| | | | | | liquid | | | | | | |
| T0 | 2 | 5.7 | 12 | ≤B9, ≤BY7; ≤Y7 | slightly | 7 | 0 | 162.0 | 0.00 | 0.90 | 99.10 |
| | | | | | opalescent | | | | | | |
| | | | | | liquid | | | | | | |
| T3 m | | 5.7 | 10.6 | ≤B4; ≤BY4; ≤Y4 | slightly | 3 | 0 | 165.5 | 0.03 | 1.07 | 98.90 |
| | | | | | opalescent | | | | | | |
| | | | | | liquid | | | | | | |
| T6 m | | 5.69 | 10.7 | ≤B9, ≤BY7, ≤Y7 | slightly | 6 | 0 | 167.8 | 0.07 | 1.12 | 98.81 |
| | | | | | opalescent | | | | | | |
| | | | | | liquid | | | | | | |
| T0 | 3 | 5.7 | 4.51 | ≤B9, ≤BY7, ≤Y7 | clear liquid | 2 | 0 | 166.2 | 0.00 | 1.00 | 98.90 |
| T3 m | | 5.6 | 4.7 | ≤B4; ≤BY4; ≤Y4 | clear liquid | 1 | 0 | 163.9 | 0.04 | 1.23 | 98.70 |
| T6 m | | 5.68 | 4.09 | ≤B6, ≤BY5, ≤Y5 | clear liquid | 16 | 2 | 163.7 | 0.09 | 1.28 | 98.64 |

| | | | cSDS | | | | | | |
|---|---|---|---|---|---|---|---|---|---|
| | | cSDS | (Non- | cIEF | | | PS80 | EGFR Binding | cMET Binding |
| Time- | Formu- | (Reduced) | Reduced) | Sum of Acidic | Main | Sum of Basic | % | % activity | % activity |
| point | lation | Purity % | Purity % | Peaks % | Peak % | Peaks % | (w/v) | relative to RM | relative to RM |
| T0 | 1 | 94.98 | 98.09 | 21.2 | 76.1 | 2.6 | 0.058 | 103 | 87 |
| T3 m | | 99.00 | 98.06 | 23.4 | 73.5 | 3.0 | 0.06 | 102 | 100 |
| T6 m | | 95.00 | 97.93 | 22.7 | 75.0 | 2.3 | 0.058 | | |
| T0 | 2 | 95.04 | 98.14 | 22.0 | 75.4 | 2.6 | 0.056 | 103 | 102 |
| T3 m | | 99.06 | 98.20 | 22.7 | 74.5 | 2.8 | 0.06 | 96 | 98 |
| T6 m | | 94.87 | 98.09 | 21.9 | 75.6 | 2.4 | 0.055 | | |
| T0 | 3 | 94.86 | 98.23 | 20.6 | 76.3 | 3.2 | 0.058 | 103 | 94 |
| T3 m | | 98.87 | 98.15 | 23.2 | 73.8 | 3.0 | 0.06 | 87 | 109 |
| T6 m | | 94.77 | 97.93 | 22.3 | 74.0 | 3.7 | 0.052 | | |

TABLE 8
Stability Study 25° C.

| | | | | | | Particulate Matter | | | SE-HPLC | | |
|---|---|---|---|---|---|---|---|---|---|---|---|
| | | | | | | (Sub-visible) | | | | | Main |
| Time- | Formu- | | Turbidity | | | ≥10 μm | ≥25 μm | A280 | LMWS | HMWS | Component |
| point | lation | pH | NTU | Color | Appearance | per mL | per mL | mg/mL | % | % | % |
| T0 | 1 | 6.1 | 5.9 | ≤B9, ≤BY7, ≤Y7 | clear liquid | 3 | 0 | 163.5 | 0.00 | 1.20 | 98.80 |
| T3 m | | 6 | 6.2 | ≤B5; ≤BY4; ≤Y4 | slightly | 6 | 0 | 163.3 | 0.23 | 2.04 | 97.70 |
| | | | | | opalescent | | | | | | |
| | | | | | liquid | | | | | | |
| T6 m | | 6.02 | 6.14 | ≤B9, ≤BY7, ≤Y7 | slightly | 8 | 1 | 161.4 | 0.58 | 2.66 | 96.77 |
| | | | | | opalescent | | | | | | |
| | | | | | liquid | | | | | | |
| T0 | 2 | 5.7 | 12 | ≤B9, ≤BY7, ≤Y7 | slightly | 7 | 0 | 162.0 | 0.00 | 0.90 | 99.10 |
| | | | | | opalescent | | | | | | |
| | | | | | liquid | | | | | | |
| T3 m | | 5.7 | 11.6 | ≤B4; ≤BY4; ≤Y4 | slightly | 4 | 0 | 166.7 | 0.18 | 1.52 | 98.36 |
| | | | | | opalescent | | | | | | |
| | | | | | liquid | | | | | | |
| T6 m | | 5.7 | 10.9 | ≤B9, ≤BY7, ≤Y7 | slightly | 125 | 2 | 166.6 | 0.50 | 1.81 | 97.69 |
| | | | | | opalescent | | | | | | |
| | | | | | liquid | | | | | | |
| T0 | 3 | 5.7 | 4.51 | ≤B9, ≤BY7, ≤Y7 | clear liquid | 2 | 0 | 166.2 | 0.00 | 1.00 | 98.90 |
| T3 m | | 5.7 | 4.1 | ≤B4; ≤BY4; ≤Y4 | clear liquid | 2 | 0 | 164.7 | 0.22 | 1.79 | 98.00 |
| T6 m | | 5.69 | 4.44 | ≤B6, ≤BY5, ≤Y5 | clear liquid | 16 | 2 | 166.5 | 0.61 | 2.09 | 97.29 |

| | | | cSDS | | | | | | |
|---|---|---|---|---|---|---|---|---|---|
| | | cSDS | (Non- | cIEF | | | PS80 | EGFR Binding | cMET Binding |
| Time- | Formu- | (Reduced) | Reduced) | Sum of Acidic | Main | Sum of Basic | % | % activity | % activity |
| point | lation | Purity % | Purity % | Peaks % | Peak % | Peaks % | (w/v) | relative to RM | relative to RM |
| T0 | 1 | 94.98 | 98.09 | 21.2 | 76.1 | 2.6 | 0.058 | 103 | 87 |
| T3 m | | 98.46 | 96.68 | 45.8 | 50.5 | 3.7 | 0.06 | 100 | 94 |
| T6 m | | 93.49 | 95.35 | 75.3 | 22.0 | 2.7 | 0.052 | | |
| T0 | 2 | 95.04 | 98.14 | 22.0 | 75.4 | 2.6 | 0.056 | 103 | 102 |
| T3 m | | 98.27 | 96.81 | 34.5 | 60.9 | 4.5 | 0.05 | 77 | 89 |
| T6 m | | 92.84 | 96.06 | 41.8 | 53.2 | 5.0 | 0.048 | | |
| T0 | 3 | 94.86 | 98.23 | 20.6 | 76.3 | 3.2 | 0.058 | 103 | 94 |
| T3 m | | 98.35 | 96.79 | 33.5 | 61.4 | 5.1 | 0.05 | 92 | 105 |
| T6 m | | 93.04 | 95.61 | 44.2 | 50.9 | 4.9 | 0.044 | | |

TABLE 9
Stability Study 40° C.

| | | | | | | Particulate Matter | | | SE-HPLC | | |
|---|---|---|---|---|---|---|---|---|---|---|---|
| | | | | | | (Sub-visible) | | | | | Main |
| Time- | Formu- | | Turbidity | | | ≥10 μm | ≥25 μm | A280 | LMWS | HMWS | Component |
| point | lation | pH | NTU | Color | Appearance | per mL | per mL | mg/mL | % | % | % |
| T0 | 1 | 6.1 | 5.9 | ≤B9, ≤BY7, ≤Y7 | clear liquid | 3 | 0 | 163 | 0.0 | 1.20 | 98.80 |
| T1 m | | 6 | 6.3 | ≤B5; ≤BY4; ≤ Y4 | slightly | 5 | 0 | 161 | 0.6 | 3.00 | 96.40 |
| | | | | | opalescent | | | | | | |
| | | | | | liquid | | | | | | |
| T3 m | | 5.9 | 8.7 | ≤B3; ≤BY3; ≤Y4 | slightly | 3 | 0 | 164 | 1.5 | 11.60 | 86.90 |
| | | | | | opalescent | | | | | | |
| | | | | | liquid | | | | | | |
| T6 m | | 5.66 | 9.4 | ≤B1, ≤BY1, ≤Y1 | slightly | 13.33 | 1 | 165 | 3.0 | 29.70 | 67.26 |
| | | | | | opalescent | | | | | | |
| | | | | | liquid | | | | | | |
| T0 | 2 | 5.7 | 12 | ≤B9, ≤BY7, ≤Y7 | slightly | 7 | 0 | 162 | 0.0 | 0.90 | 99.10 |
| | | | | | opalescent | | | | | | |
| | | | | | liquid | | | | | | |
| T1 m | | 5.7 | 10.6 | ≤B4; ≤BY4; ≤Y4 | slightly | 0 | 0 | 164 | 0.6 | 2.10 | 97.30 |
| | | | | | opalescent | | | | | | |
| | | | | | liquid | | | | | | |
| T3 m | | 5.7 | 11.9 | ≤B5; ≤BY5; ≤Y5 | slightly | 5 | 0 | 167 | 1.5 | 4.58 | 93.90 |
| | | | | | opalescent | | | | | | |
| | | | | | liquid | | | | | | |
| T6 m | | 5.69 | 15 | ≤B4, ≤BY3, ≤Y3 | slightly | 17 | 0 | 168 | 3.6 | 8.54 | 87.82 |
| | | | | | opalescent | | | | | | |
| | | | | | liquid | | | | | | |
| T0 | 3 | 5.7 | 4.5 | ≤B9, ≤BY7, ≤Y7 | clear liquid | 2 | 0 | 166 | 0.0 | 1.00 | 98.90 |
| T1 m | | 5.6 | 4.7 | ≤B4; ≤BY4; ≤Y4 | clear liquid | 1 | 0 | 159 | 0.7 | 2.60 | 96.80 |
| T3 m | | 5.6 | 5 | ≤B6; ≤BY6; ≤Y6 | clear liquid | 3 | 0 | 162 | 1.6 | 5.44 | 93.00 |
| T6 m | | 5.68 | 6.32 | ≤B5, ≤BY4, ≤Y4 | clear liquid | 0 | 0 | 165 | 4.0 | 10.78 | 85.20 |

| | | | cSDS | | | | | | |
|---|---|---|---|---|---|---|---|---|---|
| | | cSDS | (Non- | cIEF | | | PS80 | EGFR Binding | cMET Binding |
| Time- | Formu- | (Reduced) | Reduced | Sum of Acidic | Main | Sum of Basic | % | % activity | % activity relative |
| point | lation | Purity % | Purity % | Peaks % | Peak % | Peaks % | (w/v) | relative to RM | to RM |
| T0 | 1 | 94.98 | 98.09 | 21.21 | 76.1 | 2.6 | 0.1 | 103.000 | 87 |
| T1 m | | 92.96 | 94.62 | 68.35 | 26.6 | 5.1 | 0.1 | 69.000 | 96 |
| T3 m | | 92.10 | 85.76 | 97.41 | 2.2 | 0.4 | 0.1 | 41.00 | 67 |
| T6 m | | 82.37 | 70.16 | 100.00 | 0.0 | 0.0 | 0.1 | | |
| T0 | 2 | 95.04 | 98.14 | 21.97 | 75.4 | 2.6 | 0.1 | 103.000 | 102 |
| T1 m | | 91.69 | 95.26 | 52.25 | 41.3 | 6.5 | 0.1 | 68.000 | 90 |
| T3 m | | 91.11 | 79.99 | 79.77 | 15.6 | 4.6 | 0.0 | 38.00 | 77 |
| T6 m | | 82.25 | 70.75 | 92.22 | 5.9 | 1.8 | 0.0 | | |
| T0 | 3 | 94.86 | 98.23 | 20.58 | 76.3 | 3.2 | 0.1 | 103.000 | 94 |
| T1 m | | 92.74 | 94.65 | 53.89 | 40.8 | 5.3 | 0.1 | 76.000 | 100 |
| T3 m | | 92.57 | 82.36 | 80.92 | 15.0 | 4.1 | 0.1 | 46.00 | 85 |
| T6 m | | 83.70 | 72.89 | 90.41 | 8.0 | 1.6 | 0.0 | | |

TABLE 10
Post Translational Modification-Oxidation Site

| | | | | | anti-EGFR | anti-EGFR | anti-EGFR |
|---|---|---|---|---|---|---|---|
| | | anti-EGFR | anti-EGFR | anti-EGFR | HC Met 260/ | HC Met 436/ | n/a/anti-c- |
| | | HC Met 34*/ | HC Met 103*/ | HC Met 108*/ | anti-c-Met | anti-c-Met | Met LC |
| | | anti-c-Met | anti-c-Met | anti-c-Met | HC Met | HC Met | Trp 32*, |
| Timepoint | Formulation | n/a (%) | n/a (%) | n/a (%) | 254 (%) | 430 (%) | Trp 35 (%) |
| T0 | 1 | 0.1 | 1.6 | 1.5 | 2.4 | 1.0 | 0.9 |
| | 2 | 0.1 | 1.5 | 1.5 | 2.3 | 1.0 | 1.0 |
| | 3 | 0.2 | 1.6 | 1.9 | 2.3 | 1.1 | 0.9 |
| T3 m | 1 | 0.7 | 1.8 | 2.0 | 2.9 | 1.2 | 0.9 |
| 5° C. | 2 | 0.4 | 1.8 | 1.7 | 2.7 | 1.0 | 1.1 |
| | 3 | 0.4 | 1.8 | 1.7 | 2.7 | 1.0 | 1.1 |
| T3 m | 1 | 0.4 | 2.4 | 2.5 | 3.3 | 1.3 | 2.2 |
| 25° C. | 2 | 0.4 | 2.5 | 2.5 | 3.3 | 1.2 | 2.6 |
| | 3 | 0.4 | 2.5 | 2.5 | 3.3 | 1.2 | 2.6 |
| T3 m | 1 | 0.3 | 9.5 | 11.5 | 8.2 | 2.9 | 13.9 |
| 40° C. | 2 | 0.6 | 5.2 | 5.6 | 5.1 | 1.9 | 9.7 |
| | 3 | 0.6 | 5.2 | 5.6 | 5.1 | 1.9 | 9.7 |

TABLE 11
Post Translational Modification-Deamidation and Isomerization Sites

| | | anti-EGFR | anti-EGFR HC | anti-EGFR | anti-EGFR | |
|---|---|---|---|---|---|---|
| | | HC Asn 333*/ | Asn 392, Asn | n/a/anti-c- | HC Asp 53*, | anti-EGFR |
| | | anti-c-Met | 397/anti-c-Met | Met HC | Asp 54*/ | HC Asp 99*/ |
| | | HC Asn | HC Asn 386, | Asn 55*, | anti-c-Met | anti-c-Met |
| Timepoint | Formulation | 327 (%) | Asn 391 (%) | 59* (%) | n/a (%) | n/a (%) |
| T0 | 1 | 0.5 | 2.5 | 4.1 | 0.5 | 1.0 |
| | 2 | 0.6 | 2.5 | 4.0 | 0.7 | 1.1 |
| | 3 | 0.6 | 2.2 | 3.5 | 0.5 | 1.1 |
| T3 m | 1 | 0.6 | 3.0 | 4.4 | 0.5 | 1.2 |
| 5 C. | 2 | 0.7 | 2.8 | 4.5 | 0.7 | 1.3 |
| | 3 | 0.7 | 2.8 | 4.5 | 0.7 | 1.3 |
| T3 m | 1 | 3.1 | 3.5 | 6.0 | 1.1 | 3.3 |
| 25 C. | 2 | 4.1 | 3.1 | 5.7 | 1.5 | 3.4 |
| | 3 | 4.1 | 3.1 | 5.7 | 1.5 | 3.4 |
| T3 m | 1 | 25.8 | 6.6 | 16.3 | 4.7 | 19.0 |
| 40 C. | 2 | 33.1 | 5.1 | 16.8 | 6.0 | 19.5 |
| | 3 | 33.1 | 5.1 | 16.8 | 6.0 | 19.5 |

### Example 3: Polysorbate Concentration Range Shaking and Freeze Thaw Study

This study is conducted to determine the range of polysorbate 80 (PS80) concentration that stabilizes amivantamab from mechanical, interfacial, and freeze/thaw stress. The study also evaluates the protective properties of polysorbate 80 after storage for 12 months at 5° C.

Multiple, identical sets of test formulation vials are created. Each set contains two vials each of test formulations containing polysorbate 80 at concentrations below, at, and above the target (0.06% w/v) PS80 value. The set will also include test formulation control vials that do not contain PS80. All other formulation components are held constant (160 mg/mL amivantamab, 30 mM Acetate, 8.5% sucrose, 1 mg/mL methionine, 20 μg/mL ethylenediaminetetraacetic acid (EDTA) at pH 5.7). The formulations are dispensed to a fill volume of 7.1 mL into 8R vials, stoppered, capped, and crimp sealed.

To establish general study baseline data, one set of vials is tested at the start of the study to serve as an untreated, Time Zero Control (T=0).

To evaluate the stabilizing effect of polysorbate 80 against mechanical and interfacial stress, one set of vials is placed horizontally on an orbital shaker and shaken at approximately 250 rpm for up to 72 hours under ambient room temperature and light conditions (T72h Shaking). During the same time period, a second corresponding unshaken control set of vials is held vertically at ambient room and light conditions (T72h Control).

To evaluate the stabilizing effect of aged polysorbate 80 against mechanical and interfacial stress, two sets of vials are held for 12 months at 5° C. Using the methods stated above, one set is shaken for up to 72 hrs (T12m T72h Shaking), the other set held as a control (T12m T72h Control).

To evaluate the stabilizing effect of polysorbate 80 against freeze/thaw stress, one set of vials are subjected to five (5) freeze/thaw cycles (5×FT) with one cycle defined as freezing to −70° C. followed by passive thawing at ambient room temperature.

All study samples will be evaluated for stability by Color, pH, Turbidity, Particulate Matter (Sub-visible), Protein Concentration (A280), SE-HPLC, cSDS (Reduced), cSDS (Non-Reduced), cIEF, PS80, potency (EGFR), potency (cMET).

It is anticipated that shaken no-PS80 control samples will show an attribute-value profile consistent with the presence of agitation-induced degradation including an increased detection of aggregates. It is also anticipated that samples containing very low levels of PS80 will show a sharp decrease in agitation-induced degradation to values just above or equal to the attribute-value profile seen in T=0 and corresponding unshaken control sample. It is further anticipated that samples containing low, target, and high levels of PS80 will exhibit an attribute-value profile highly similar to T=0 and corresponding unshaken control sample. This data demonstrates PS80's ability to stabilize amivantamab from mechanical and interfacial stress.

Similar results are anticipated for samples held for 12 months at 5° C. prior to shaking stress. Also, no substantial differences in attribute values are anticipated after freeze/thaw stress versus T=0 controls.

Under both shaking stress and freeze/thaw stress, it is anticipated that there will be no substantial differences in attribute values between the low, target, and high polysorbate 80 samples. This indicates that stable amivantamab formulated with low, target and high polysorbate 80 concentrations levels protect against mechanical, interfacial, and freeze/thaw stress.

### Evaluation of Polysorbate 80 Containing Formulations Aged Under Accelerated Conditions Against Shaking and Freeze/Thaw Stresses

This study was conducted to evaluate a test formulation of 160 mg/mL amivantamab formulated with polysorbate 80 against mechanical, interfacial, and freeze/thaw stress when aged for six months under accelerated conditions.

160 mg/mL amivantamab in 10 mM acetate, 8.5% sucrose, 1 mg/mL Methionine, 20 g/mL EDTA, 0.06% PS-80, pH 5.1 was aliquoted into multiple replicate 30R vials at a fill volume of 15.6 mL per vial. The vials were stoppered, capped, and crimp sealed. To establish general study baseline data, one replicate vial was tested at the start of the study to serve as a Time Zero Control (T=0). The remaining replicate vials were placed on stability at accelerated (25° C.) conditions for six months.

To evaluate the stability of the aged formulation against mechanical and interfacial stress, an aged replicate vial was placed horizontally on an orbital shaker and shaken at approximately 250 rpm for up to 72 hours under ambient room temperature (+shaking).

To evaluate the stability of the aged formulation against freeze/thaw stress, an aged replicate vial was subjected to five (5) freeze/thaw cycles (5×FT) with one cycle defined as freezing to −70° C. for 24 hours followed by passive thawing at ambient room temperature for 24 hours.

A remaining aged replicate vial was used as an untreated control for the Shaking and Freeze/thaw study.

All study samples were evaluated for stability by Color, pH, Turbidity, Particulate Matter (Sub-visible), Protein Concentration (A280), SE-HPLC, cSDS (Reduced), cSDS (Non-Reduced), cIEF, PS80, potency (EGFR), potency (cMET).

The study results are listed in Table 11A below. Comparison of the T=0 and untreated control samples aged for six months under accelerated conditions showed slight to minimal changes in attribute values consistent with protein degradation over 6 months at 25° C. Of particular note, all aged samples showed an equivalent decrease in PS80 concentration (0.043-0.044%) verses T=0 (0.058%).

Comparison of the samples aged for six months under accelerated conditions showed no appreciable difference between exposure to shaking stress, repeated freeze thaw stress, or untreated control. This demonstrates that 160 mg/mL amivantamab formulated with 0.04% to 0.06% polysorbate 80 concentrations levels protect against mechanical, interfacial, and freeze/thaw stress.

TABLE 11A
Test Formulation Aged Under Accelerated Conditions and Exposed to Shaking and Freeze/Thaw Stress

| | | | | | Particulate Matter | | | SE-HPLC | | |
|---|---|---|---|---|---|---|---|---|---|---|
| | | | | | (Sub-visible) | | | | | Main |
| | | Turbidity | | | ≥10 μm | ≥25 μm | A280 | LMWS | HMWS | Component |
| Timepoint | pH | NTU | Color | Appearance | per mL | per mL | mg/mL | % | % | % |
| T0 | 5.7 | 4.51 | ≤B9, | clear | 2 | 0 | 166.17 | 0.00 | 1.00 | 98.90 |
| | | | ≤BY7, | liquid | | | | | | |
| | | | ≤Y7 | | | | | | | |
| T6 m | 5.69 | 4.44 | ≤B6, | clear | 16 | 2 | 166.50 | 0.61 | 2.09 | 97.29 |
| | | | ≤BY5, | liquid | | | | | | |
| | | | ≤Y5 | | | | | | | |
| T6 m + | 5.59 | 4.46 | ≤B6, | clear | 9 | 1 | 167.15 | 0.55 | 2.06 | 97.39 |
| 5 × F/T | | | ≤BY5, | liquid | | | | | | |
| | | | ≤Y6 | | | | | | | |
| T6 m + | 5.6 | 4.43 | ≤B5, | clear | 18 | 2 | 166.43 | 0.53 | 2.08 | 97.39 |
| shaking | | | ≤BY5, | liquid | | | | | | |
| | | | ≤Y5 | | | | | | | |

| | |
|---|---|
| EGFR | cMET |

| | | cSDS | cIEF | | | | Binding | Binding |
|---|---|---|---|---|---|---|---|---|
| | cSDS | (Non- | Sum of | | Sum of | | % | % |
| | (Reduced) | Reduced) | Acidic | Main | Basic | PS80 | activity | activity |
| | Purity | Purity | Peaks | Peak | Peaks | % | relative | relative |
| Timepoint | % | % | % | % | % | (w/v) | to RM | to RM |
| T0 | 94.86 | 98.23 | 20.6 | 76.3 | 3.2 | 0.058 | 103 | 94 |
| T6 m | 93.04 | 95.61 | 44.2 | 50.9 | 4.9 | 0.044 | 83 | 99 |
| T6 m + | 93.29 | 95.50 | 39.8 | 54.6 | 5.6 | 0.043 | 95 | 84 |
| 5 × F/T | | | | | | | | |
| T6 m + | 92.89 | 95.41 | 38.6 | 56.1 | 5.4 | 0.044 | 86 | 97 |
| shaking | | | | | | | | |

### Example 4: Polysorbate Concentration Range Shaking & Freeze Thaw in rHuPH20 Containing Formulations

This study is conducted to determine the range of polysorbate 80 (PS80) concentration that stabilizes amivantamab with rHuPH20 from mechanical, interfacial, and freeze/thaw stress. The study also evaluates the protective properties of polysorbate 80 after storage for 12 months at 5° C.

Multiple, identical sets of test formulation vials are created. Each set contains two vials each of test formulations containing polysorbate 80 at concentrations below, at, and above the target (0.06% w/v) PS80 value. The set will also include test formulation control vials that do not contain PS80. All other formulation components are held constant (160 mg/mL amivantamab, 30 mM Acetate 8.5% sucrose, 1 mg/mL methionine, 20 μg/mL ethylenediaminetetraacetic acid (EDTA), 2000 U/mL rHuPH20 at pH 5.7). The formulations are dispensed to a fill volume of 7.1 mL into 8R vials, stoppered, capped, and crimp sealed.

To establish general study baseline data, one set of vials is tested at the start of the study to serve as an untreated, Time Zero Control (T=0).

To evaluate the stabilizing effect of polysorbate 80 against mechanical and interfacial stress, one set of vials is placed horizontally on an orbital shaker and shaken at approximately 250 rpm for up to 72 hours under ambient room temperature and light conditions (T72h Shaking). During the same time period, a second corresponding unshaken control set of vials is held vertically at ambient room and light conditions (T72h Control).

To evaluate the stabilizing effect of aged polysorbate 80 against mechanical and interfacial stress, two sets of vials are held for 12 months at 5° C. Using the methods stated above, one set is shaken for up to 72 hrs (T12m T72h Shaking), the other set held as a control (T12m T72h Control).

To evaluate the stabilizing effect of polysorbate 80 against freeze/thaw stress, one set of vials are subjected to five (5) freeze/thaw cycles (5×FT) with one cycle defined as freezing to −70° C. followed by passive thawing at ambient room temperature.

All study samples will be evaluated for stability by Color, rHuPH20 activity, pH, Turbidity, Particulate Matter (Sub-visible), Protein Concentration (A280), SE-HPLC, cSDS (Reduced), cSDS (Non-Reduced), cIEF, PS80, potency (EGFR), potency (cMET). It is anticipated that shaken no-PS80 control samples will show an attribute-value profile consistent with the presence of agitation-induced degradation including an increased detection of aggregates. It is also anticipated that samples containing very low levels of PS80 will show a sharp decrease in agitation-induced degradation to values just above or equal to the attribute-value profile seen in T=0 and corresponding unshaken control sample. It is further anticipated that samples containing low, target, and high levels of PS80 will exhibit an attribute-value profile highly similar to T=0 and corresponding unshaken control sample. This data demonstrates PS80's ability to stabilizes amivantamab from mechanical and interfacial stress.

Similar results are anticipated for samples held for 12 months at 5° C. prior to shaking stress. Also, no substantial differences in attribute values are anticipated after freeze/thaw stress versus T=0 controls.

Under both shaking stress and freeze/thaw stress, it is anticipated that there will be no substantial differences in attribute values between the low, target, and high polysorbate 80 samples. This indicates that stable amivantamab formulated with low, target and high polysorbate 80 concentrations levels protect against mechanical, interfacial, and freeze/thaw stress.

### Example 5: Formulation Robustness Development

### Study Design

A study is performed to examine the effects of multi-factorial varying of formulation component concentration levels of bispecific EGFR-cMet antibody drug product held at recommended (5° C.) and accelerated (25° C.) conditions. The formulation components evaluated are protein concentration, acetate concentration, sucrose concentration, polysorbate 80 concentration, EDTA/methionine concentration, and pH level. The lowest and highest test factor concentration values tested are approximately 10 to 40% lower or higher than the target test factor concentration value respectively (see Table 12).

TABLE 12
Ranges of the factor concentrations tested

| Test Factors | Low | Target | High |
|---|---|---|---|
| Bispecific EGFR-cMet antibody | 144 | 160 | 176 |
| (amivantamab) protein | | | |
| concentration (mg/mL) | | | |
| pH | 5.2 | 5.7 | 6.2 |
| Acetate concentration (mM) | 20 | 30 | 40 |
| Sucrose concentration (% w/v) | 6.8 | 8.5 | 10.2 |
| EDTA concentration (μg/mL) | 16 | 20 | 24 |
| Methionine concentration (mg/mL) | 0.8 | 1 | 1.2 |
| PS80 concentration (% w/v) | 0.036 | 0.06 | 0.084 |

Based on this criterion, statistical software was used to create a Multi-Factorial Design of Experiments statistical model designating the number and compositions of test formulations required for the study.

Test formulations are prepared and aliquoted into 8R vials at a fill volume of 7.1 mL. The vials are stoppered, capped, and crimp sealed. The vials are placed on stability at recommended (5° C.) and accelerated (25° C.) conditions. At designated time points, samples are pulled and assayed.

### Study Results

The test results for each attribute of the test formulations at study initiation (time zero), after 12 months at the recommended storage condition (5° C.), and after 6 months at accelerated temperature (25° C.) will be presented in tabular form reporting the range, mean, and standard deviation of the test formulations for each of the following attributes: cIEF (% area Main Peak, % Sum of Acidic peaks, % Sum of Basic peaks), cSDS (% Purity (non reduced), % Purity (reduced)), SE-HPLC (% Aggregate, % Monomer, % Fragment), Particulate Matter (sub-vis) (Particles/container 10 m, Particles/container 25 m), and Turbidity (NTU).

It is anticipated that the analytical results for all formulations held for 12 months at 5° C. will demonstrate little changes in the assay test values indicating stability. The ability for all formulations with multi-variant ranges in excipient concentrations to yield a narrow range of assay test result values demonstrates the robustness of the formulation within the boundaries and storage conditions tested. Additionally, it is anticipated that the full range of values observed per assay in this study will be consistent with the most preferred embodiment of stability when held at 2-8° C.

It is also anticipated that the analytical results for all formulations held for six months at accelerated (25° C.) storage conditions will show degradation effects consistent with the stability profile of bispecific EGFR-cMet antibody exposed to prolonged accelerated storage conditions. However, for most results, the magnitude of the effect will be relatively minor compared to results seen at 12 months at 5° C. Similarly, the magnitude of increase in the range of result values should also be relatively minor with some of the ranges being equivalent, or less than, those seen at 12 months at 5° C. This demonstrates that even under accelerated storage conditions the multi-variant ranges in excipient concentration yield relatively consistent results.

### Example 6: Formulation Robustness Development with rHuPH20

### Study Design

A study is performed to examine the effects of multi-factorial varying of formulation component concentration levels of bispecific EGFR-cMet antibody drug product with rHuPH20 held at recommended (5° C.) and accelerated (25° C.) conditions. The formulation components evaluated are protein concentration, acetate concentration, sucrose concentration, polysorbate 80 concentration, EDTA/methionine concentration, rHuPH20 concentration and pH. The lowest and highest test factor concentration values tested are approximately 10 to 50% lower or higher than the target test factor concentration value respectively (see Table 13).

TABLE 13
Ranges of the factor concentrations tested

| Test Factors | Low | Target | High |
|---|---|---|---|
| Bispecific EGFR-cMet antibody | 144 | 160 | 176 |
| (amivantamab) protein | | | |
| concentration (mg/mL) | | | |
| pH | 5.2 | 5.7 | 6.2 |
| Acetate concentration (mM) | 20 | 30 | 40 |
| Sucrose concentration (% w/v) | 6.8 | 8.5 | 10.2 |
| EDTA concentration (μg/mL) | 16 | 20 | 24 |
| Methionine concentration (mg/mL) | 0.8 | 1 | 1.2 |
| PS80 concentration (% w/v) | 0.036 | 0.06 | 0.084 |
| rHuPH20 (U/mL) | 1000 | 2000 | 3000 |

Based on this criterion, statistical software was used to create a Multi-Factorial Design of Experiments statistical model designating the number and compositions of test formulations required for the study.

Test formulations are prepared and aliquoted into 8R vials at a fill volume of 7.5 mL. The vials are stoppered, capped, and crimp sealed. The vials are placed on stability at recommended (5° C.) and accelerated (25° C.) conditions. At designated time points, samples are pulled and assayed.

### Study Results

The test results for each attribute of the test formulations at study initiation (time zero), after 12 months at the recommended storage condition (5° C.), and after 6 months at accelerated temperature (25° C.) will be presented in tabular form reporting the range, mean, and standard deviation of the test formulations for each of the following attributes: cIEF (% area Main Peak, % Sum of Acidic peaks, % Sum of Basic peaks), cSDS (% Purity (non reduced), % Purity (reduced)), SE-HPLC (% Aggregate, % Monomer, % Fragment), Particulate Matter (sub-vis) (Particles/container 10 m, Particles/container 25 m), Turbidity (NTU), and rHuPH20 Activity (U/mL).

It is anticipated that the analytical results for all formulations held for 12 months at 5° C. will demonstrate little changes in the assay test values indicating stability. The ability for all formulations with multi-variant ranges in excipient concentrations to yield a narrow range of assay test result values demonstrates the robustness of the formulation within the boundaries and storage conditions tested. Additionally, it is anticipated that the full range of values observed per assay in this study will be consistent with the most preferred embodiment of stability when held at 2-8° C.

It is also anticipated that the analytical results for all formulations held for six months at accelerated (25° C.) storage conditions will show degradation effects consistent with the stability profile of bispecific EGFR-cMet antibody exposed to prolonged accelerated storage conditions. However, for most results, the magnitude of the effect will be relatively minor compared to results seen at 12 months at 5° C. Similarly, the magnitude of increase in the range of result values should also be relatively minor with some of the ranges being equivalent, or less than, those seen at 12 months at 5° C. This demonstrates that even under accelerated storage conditions the multi-variant ranges in excipient concentration yield relatively consistent results.

### Example 7: Formulated Drug Bulk Production

### Process Description

### Processing Solutions

TABLE 14
Processing Solutions Target Composition and Ranges

| Solution | Composition and Ranges |
|---|---|
| Diafiltration | 10 mM Acetate, 8.5% sucrose, |
| Buffer | 1 mg/mL L-methionine, pH 5.2 ± 0.3 |
| Polysorbate 80, | 10 mM Acetate, 8.5% sucrose, |
| EDTA | 1 mg/mL L-methionine, 6.0% (w/v) |
| Stock Solution | Polysorbate 80, 2 mg/mL EDTA, pH 5.2 ± 0.3 |

### Ultrafiltration/Diafiltration (UF/DF)

Ultrafiltration/diafiltration (UF/DF) is performed to re-formulate the amivantamab Virus Retentive filtrate intermediate manufacturing solution to a pre-formulated bulk (pFB) solution consisting of 160 mg/mL amivantamab, 30 mM Acetate, 8.5% sucrose, 1 mg/mL L-methionine, pH 5.7. Note that during the UF/DF operation the acetate concentration of the diafiltration buffer increases from 10 mM to 30 mM due to the Gibbs-Donnan effect. The pH also shifts from 5.2 to 5.7.

### Preparation of Amivantamab Formulated Bulk (FB)

Polysorbate 80 (6.0% w/v) and EDTA (2 mg/mL) stock solution is added to the pFB at a 1:100 dilution to obtain a final concentration of 0.06% (w/v) Polysorbate 80, and 20 μg/mL EDTA yielding the Formulated Bulk (FB) consisting of 160 mg/mL amivantamab in 30 mM Acetate, 8.5% (w/v) sucrose, 1 mg/mL L-methionine, 0.06% Polysorbate 80, 20 μg/mL EDTA, pH 5.7. The FB solution is then mixed uniformly. Final filtration of the Formulated Bulk is achieved using a sterile 0.45/0.22 μm filter immediately followed with a subsequent, in-line 0.22 μm filter.

### Final Bulk Fill

Following final filtration, the FB is filled into polycarbonate Biotainer(s). The fill volume is 20% to 90% of the biotainer's stated volume.

### Final Bulk Storage and Shipping

Storage and Shipment Conditions of the Formulated Bulk Prior to Drug Product production is 5° C.±3° C. protected from light if FB is stored for about one week or less or −40° C.±10° C. protected from light if FB is stored for more than one week.

### Example 8: Drug Formulation with rHuPh20 Production

### Process Description

### Processing Solutions

TABLE 15
Processing Solutions Target Composition and Ranges

| Solution | Composition and Ranges |
|---|---|
| Amivantamab | 160 mg/mL amivantamab, 30 mM Acetate, |
| Formulated Bulk | 8.5% (w/v) sucrose, 1 mg/mL L-methionine, |
| | 0.06% Polysorbate 80, 20 pg/mL EDTA, pH 5.7 |
| rHuPH20 | 10 mg/mL rHuPH20⁽¹⁾ 10 mM Histidine, |
| Formulated Bulk | 130 mM NaCl, pH 6.5 |

⁽¹⁾100,000 IU/mg rHuPH20

Preparation of Amivantamab Drug Formulation with rHuPH20

rHuPH20 Formulated Bulk is added to the Amivantamab Formulated Bulk solution to obtain a final concentration of 2,000 IU/mL rHuPH20 yielding the Drug Formulation with rHuPH20 consisting of 160 mg/mL amivantamab in 30 mM Acetate, 8.5% (w/v) sucrose, 1 mg/mL L-methionine, 0.06% Polysorbate 80, 20 μg/mL EDTA, rHuPH20 2000IU, pH 5.7. The Histidine and sodium chloride contribution from rHuPH20 Formulated Bulk is considered minimal and therefore not listed in the Amivantamab with rHuPH20 Drug Formulation composition. Drug Formulation solution is then mixed uniformly. Initial filtration of the Drug Formulation is achieved using a sterile 0.22 μm filter. Final filtration of the Drug Formulation is achieved using a sterile 0.22 μm filter immediately followed with a subsequent, in-line 0.22 μm filter.

### Example 9: Drug Product: Composition and Components of Primary Packaging

Provided herein is a tabular summary of the composition of the Amivantamab Drug Product (Table 16).

TABLE 16
Composition of Amivantamab Drug Product

| | | | Amount |
|---|---|---|---|
| Component | Composition | | per mL |
| Amivantamab | 160 | mg | 160 mg |
| Glacial Acetic Acid | 30 | mM | 0.186 mg |
| Sodium Acetate Trihydrate | | | 3.662 mg |
| Sucrose | 8.5% | (w/v) | 85 mg |
| Polysorbate 80 | 0.06% | | 0.60 mg |
| L-Methionine | 1.0 | mg/mL | 1.0 mg |
| EDTA Disodium salt, Dihydrate | 20 | μg/mL | 0.02 mg |

| | | |
|---|---|---|
| Water for Injection | q.s to 1.0 mL | q.s to 1.0 mL |

Amivantamab drug product (DP) primary packaging consists of a glass vial, a polymer vial stopper, and an aluminum seal. Table 17 lists specific components for the primary packaging material.

TABLE 17
Primary packaging material components

| Component | Description |
|---|---|
| Glass vial | 8 mL glass Type 1 borosilicate |
| Stopper | 20 mm butyl rubber, FluroTec coated stopper |
| Seals | 20 mm aluminum seal with Flip-Off button |

### Example 10: Drug Product with rHuPH20: Composition and Components of Primary Packaging

Provided herein is a tabular summary of the composition of the Amivantamab with rHuPH20 Drug Product (Table 18).

TABLE 18
Composition of Amivantamab with rHuPH20 Drug Product

| | | | Amount |
|---|---|---|---|
| Component | Composition | | per mL |
| Amivantamab | 160 | mg | 160 mg |
| Glacial Acetic Acid | 30 | mM | 0.186 mg |
| Sodium Acetate Trihydrate | | | 3.662 mg |
| Sucrose | 8.5% | (w/v) | 85 mg |
| Polysorbate 80 | 0.06% | | 0.60 mg |
| L-Methionine | 1.0 | mg/mL | 1.0 mg |
| EDTA Disodium salt, Dihydrate | 20 | μg/mL | 0.02 mg |
| rHuPH20 (Hyaluronidase) | 2000 | IU | 0.02 mg |
| | | | (2000 U/mL) |

| | | |
|---|---|---|
| Water for Injection | q.s to 1.0 mL | q.s to 1.0 mL |

Amivantamab with rHuPH20 drug product (DP) primary packaging consists of a glass vial, a polymer vial stopper, and an aluminum seal. Table 19 lists specific components for the primary packaging material.

TABLE 19
Primary packaging material components

| Component | Description |
|---|---|
| Glass vial | 8 mL glass Type 1 borosilicate |
| Stopper | 20 mm butyl rubber, FluroTec coated stopper |
| Seals | 20 mm aluminum seal with Flip-Off button |

### Example 11: Description of Drug Product Stability Study

This study was conducted to monitor amivantamab DP attributes placed on stability under various environmental conditions and lengths of time. Study test articles were prepared by aliquoting Formulated Bulk into 8R vials at a fill volume of 7.1 mL. The vials were stoppered, capped, and crimp sealed.

All studies were performed with vials in an inverted orientation.

TABLE 20
Study parameters

| Stability | Storage | Duration |
|---|---|---|
| Classification | condition | (Months) |
| Real-time | 5 ± 3° C. | 36 |
| Accelerated | 25 ± 2° C./60% RH | 6 |
| Stressed | 40 ± 2^{o} C./75% RH | 3 |

### Stability Study Results

The stability results to date for amivantamab DP held under recommended, accelerated, and stressed conditions are listed below in Tables 21-23. It is anticipated that at all-time points for DP held at recommended storage conditions, all test parameter result values observed per assay study will exceed the criteria consistent with the most preferred embodiment of the stability when held after storage for about 12 months or more and at a temperature of about 5° C., after storage for about 12 months or more and at a temperature of about 25° C., and/or after storage for about 2 years or more and at a temperature of about 5° C. Similarly, it is anticipated peptide map results will show little to no consequential change over time in the measured percent of post translational modification.

It is anticipated results for amivantamab DP held at accelerated and stressed conditions will show the expected rates of degradation for Drug Product exposed to prolonged accelerated and stressed storage conditions. It is also anticipated that DP held at accelerated conditions (25° C.) for 12 months will show results consistent with the preferred embodiment of the stability when held at 2-8° C. for about 2 years or more.

### 5° C. Data

TABLE 21
Stability Results for Amivantamab Drug Product Stored at 5° C.

| | | | | Particulate | | | | |
|---|---|---|---|---|---|---|---|---|
| | | | | Matter | | | | |
| | | | | Sub-visible | | cSDS (Reduced) | | |
| | | | | ≥10 μm: | ≥25 μm: | | AG | |
| | Color of | | Turbidity | particles | particles | Purity: | HC: | |
| Months | Solution | pH | (NTU) | per vial | per vial | % | % | new peaks |
| 0 | ≤BY5, | 5.6 | 4.3 | 14 | 0 | 94.4 | 4.9 | No new peak >1.0% |
| | ≤B5, | | | | | | | compared to |
| | ≤Y5 | | | | | | | Reference Material |
| 3 | ≤BY6, | 5.5 | 4.4 | 19 | 1 | 94.2 | 5.0 | No new peak >1.0% |
| | ≤B6, | | | | | | | compared to |
| | ≤Y5 | | | | | | | Reference Material |
| 5 | ≤BY6, | 5.6 | 4.0 | 63 | 36 | 94.1 | 5.0 | No new peak >1.0% |
| | ≤B6, | | | | | | | compared to |
| | ≤Y5 | | | | | | | Reference Material |
| 6 | ≤BY6, | 5.7 | 4 | 18 | 3 | 94.0 | 5.1 | No new peak >1.0% |
| | ≤B6, | | | | | | | compared to |
| | ≤Y5 | | | | | | | Reference Material |
| 9 | ≤BY5, | 5.6 | 4 | 77 | 24 | 94.1 | 5.0 | No new peak >1.0% |
| | ≤B6, | | | | | | | compared to |
| | ≤Y5 | | | | | | | Reference Material |
| 12 | ≤BY5, | 5.6 | 4 | 32 | 14 | 94.1 | 5.0 | No new peak >1.0% |
| | ≤B6, | | | | | | | compared to |
| | ≤Y5 | | | | | | | Reference Material |

| | | | SE | | | |
|---|---|---|---|---|---|---|
| | | | HPLC | Protein Conc. by A₂₈₀ | | |
| | cSDS (Non-reduced) | | Main Component | HMWS | LMWS | |
| Months | Purity (%) | New Peaks (%) | (%) | (%) | (%) | (mg/mL) |
| 0 | 98.2 | No new peak >1.0% | 98.8 | 1.2 | <0.1 | 162 |
| | | compared to | | | | |
| | | Reference Material | | | | |
| 3 | 98.4 | No new peak >1.0% | 98.6 | 1.3 | <0.1 | 162 |
| | | compared to | | | | |
| | | Reference Material | | | | |
| 5 | 98.2 | No new peak >1.0% | 98.6 | 1.4 | <0.1 | 161 |
| | | compared to | | | | |
| | | Reference Material | | | | |
| 6 | 98.2 | No new peak >1.0% | 98.5 | 1.4 | <0.10 | 164 |
| | | compared to | | | | |
| | | Reference Material | | | | |
| 9 | 98.2 | No new peak >1.0% | 98.5 | 1.4 | <0.10 | 161 |
| | | compared to | | | | |
| | | Reference Material | | | | |
| 12 | 98.2 | No new peak >1.0% | 98.5 | 1.4 | <0.10 | 162 |
| | | compared to | | | | |
| | | Reference Material | | | | |

| | | | | EGFR | cMET | |
|---|---|---|---|---|---|---|
| | cIEF | | | Binding | Binding | |
| | | Sum of | Sum of | activity | activity | |
| | Main | acidic | Basic | relative to | relative to | Polysorbate |
| | peak | peaks | peaks | Reference | Reference | 80 |
| Months | (%) | (%) | (%) | Material (%) | Material (%) | (%) |
| 0 | 75 | 23 | 3 | 89 | 85 | 0.06 |
| 3 | 74 | 23 | 3 | 74 | 97 | 0.06 |
| 5 | 73 | 24 | 3 | 85 | 108 | 0.06 |
| 6 | 74 | 23 | 3 | 84 | 86 | 0.06 |
| 9 | 72 | 25 | 3 | 90 | 101 | 0.05 |
| 12 | 73 | 24 | 3 | 86 | 98 | 0.06 |
| | Post Translational Modification-Oxidation Site | | | | | |
| | anti-EGFR | anti-EGFR | anti-EGFR | anti-EGFR | anti-EGFR | anti-EGFR |
| | HC Met | HC Met | HC Met | HC Met 260/ | HC Met 436/ | n/a/anti- |
| | 34*/anti- | 103*/anti- | 108*/anti- | anti-c-Met | anti-c-Met | c-Met LC Trp |
| | c-Met n/a | c-Met n/a | c-Met n/a | HC Met 254 | HC Met 430 | 32*, Trp 35 |
| Months | (%) | (%) | (%) | (%) | (%) | (%) |
| 0 | 0.3 | 1.7 | 1.8 | 2.8 | 1.1 | 1.1 |
| 3 | 0.3 | 1.5 | 1.5 | 2.5 | 0.9 | 0.9 |
| 5 | 0.2 | 1.5 | 2.1 | 2.5 | 1.0 | 1.1 |
| 6 | 0.1 | 1.3 | 2.7 | 2.8 | 0.6 | 0.7 |
| 9 | 0.4 | 1.6 | 1.8 | 2.5 | 1.0 | 1.3 |
| 12 | 0.1 | 0.6 | 1.5 | 2.1 | 0.7 | 0.8 |

| | Post Translational Modification-Deamidation Site | | |
|---|---|---|---|
| | anti-EGFR HC Asn | anti-EGFR HC Asn | anti-EGFR n/a/ |
| | 333*/anti-c-Met | 392/anti-c-Met HC | anti-c-Met HC |
| Months | HC Asn 327 (%) | Asn 386, Asn 391 (%) | Asn 55*, 59* (%) |
| 0 | 0.7 | 1.5 | 4.3 |
| 3 | 0.6 | 2.1 | 3.7 |
| 5 | NT | 2.8 | 4.3 |
| 6 | NT | 2.1 | 3.7 |
| 9 | NT | 3.1 | 5.2 |
| 12 | NT | 2.0 | 3.7 |

| | Post Translational Modification-Isomerization Site | |
|---|---|---|
| | anti-EGFR HC Asp 53*, | anti-EGFR HC Asp |
| Months | Asp 54*/anti-c-Metn/a (%) | 99*/anti-c-Met n/a (%) |
| 0 | 0.6 | 1.5 |
| 3 | 0.5 | 1.0 |
| 5 | 0.6 | 1.1 |
| 6 | 0.4 | 1.1 |
| 9 | 0.6 | 1.4 |
| 12 | 0.5 | 1.3 |

NT = Not tested

### 25° C. Data

TABLE 22
Stability Results for Amivantamab Drug Product Stored at 25° C.

| | | | | Particulate | | | | |
|---|---|---|---|---|---|---|---|---|
| | | | | Matter | | | | |
| | | | | Sub-visible | | | | |
| | | | | ≥10 | ≥25 | cSDS (Reduced) | | |
| | | | | μm: | μm: | | AG | |
| | Color of | | Turbidity | particles | particles | Purity: | HC: | |
| Months | Solution | pH | (NTU) | per vial | per vial | % | % | New peaks |
| 0 | ≤BY5, | 5.6 | 4.3 | 14 | 0 | 94.4 | 4.9 | No new peak |
| | ≤B5, | | | | | | | >1.0% compared |
| | ≤Y5 | | | | | | | to Reference |
| | | | | | | | | Material |
| 3 | ≤BY5, | 5.5 | 4 | 27 | 4 | 93.3 | 5.1 | No new peak |
| | ≤B6, | | | | | | | >1.0% compared |
| | ≤Y5 | | | | | | | to Reference |
| | | | | | | | | Material |
| 6 | ≤BY6, | 5.7 | 5 | 17 | 8 | 92.4 | 5.1 | No new peak |
| | ≤B6, | | | | | | | >1.0% compared |
| | ≤Y5 | | | | | | | to Reference |
| | | | | | | | | Material |

| | cSDS (Non-reduced) | | SE HPLC | | | Protein |
|---|---|---|---|---|---|---|
| | | New | Main | | | Conc. by |
| | Purity | Peaks | Component | HMWS | LMWS | A₂₈₀ |
| Months | (%) | (%) | (%) | (%) | (%) | (mg/mL) |
| 0 | 98.2 | No new | 98.8 | 1.2 | <0.1 | 162 |
| | | peak | | | | |
| | | >1.0% | | | | |
| | | compared | | | | |
| | | to | | | | |
| | | Reference | | | | |
| | | Material | | | | |
| 3 | 97.0 | No new | 97.9 | 1.9 | 0.2 | 162 |
| | | peak | | | | |
| | | >1.0% | | | | |
| | | compared | | | | |
| | | to | | | | |
| | | Reference | | | | |
| | | Material | | | | |
| 6 | 95.7 | No new | 97.3 | 2.1 | 0.5 | 162 |
| | | peak | | | | |
| | | >1.0% | | | | |
| | | compared | | | | |
| | | to | | | | |
| | | Reference | | | | |
| | | Material | | | | |
| | cIEF | | | EGFR Binding | cMET Binding | |
| | Main | Sum of | Sum of | activity relative | activity relative | |
| | peak | acidic | Basic | to Reference | to Reference | Polysorbate 80 |
| Months | (%) | peaks (%) | peaks (%) | Material (%) | Material (%) | (%) |
| 0 | 75 | 23 | 3 | 89 | 85 | 0.06 |
| 3 | 60 | 35 | 4 | 71 | 94 | 0.06 |
| 6 | 53 | 41 | 5 | 57 | 113 | 0.06 |
| | Post Translational Modification-Oxidation Site | | | | | |
| | anti-EGFR | anti-EGFR | anti-EGFR | anti-EGFR | anti-EGFR | anti-EGFR |
| | HC Met | HC Met | HC Met | HC Met 260/ | HC Met 436/ | n/a/anti- |
| | 34*/anti- | 103*/anti- | 108*/anti- | anti-c-Met | anti-c-Met | c-Met LC Trp |
| | c-Met n/a | c-Met n/a | c-Met n/a | HC Met 254 | HC Met 430 | 32*, Trp 35 |
| Months | (%) | (%) | (%) | (%) | (%) | (%) |
| 0 | 0.3 | 1.7 | 1.8 | 2.8 | 1.1 | 1.1 |
| 3 | 0.4 | 2.5 | 3.1 | 3.4 | 1.4 | 1.9 |
| 6 | 0.4 | 2.3 | 1.9 | 3.1 | 1.4 | 1.9 |

| | Post Translational Modification-Deamidation Site | | |
|---|---|---|---|
| | | anti-EGFR HC | |
| | anti-EGFR HC | Asn 392/anti- | anti-EGFR n/a/ |
| | Asn 333*/anti- | c-Met HC Asn | anti-c-Met HC |
| | c-Met HC Asn 327 | 386, Asn 391 | Asn 55*, 59* |
| Months | (%) | (%) | (%) |
| 0 | 0.7 | 1.5 | 4.3 |
| 3 | NT | 2.7 | 4.6 |
| 6 | NT | 3.4 | 6.2 |

| | Post Translational Modification-Isomerization Site | |
|---|---|---|
| | anti-EGFR HC Asp 53*, | anti-EGFR HC Asp |
| | Asp 54*/anti-c-Metn/a | 99*/anti-c-Metn/a |
| Months | (%) | (%) |
| 0 | 0.6 | 1.5 |
| 3 | 1.3 | 3.0 |
| 6 | 1.7 | 4.5 |

NT = Not tested

TABLE 23
Stability Results for Amivantamab Drug Product Stored at 40° C.

| | | | | Particulate | | | | |
|---|---|---|---|---|---|---|---|---|
| | | | | Matter | | | | |
| | | | | Sub-visible | | cSDS | | |
| | | | | ≥10 | ≥25 | (Reduced) | | |
| | | | | particles | particles | | AG | |
| | Color of | | Turbidity | per vial | per vial | Purity: | HC: | |
| Months | Solution | pH | (NTU) | μm: | μm: | % | % | New peaks |
| 0 | ≤BY5, | 5.6 | 4.3 | 14 | 0 | 94.4 | 4.9 | No new peak > |
| | ≤B5, | | | | | | | 1.0% compared |
| | ≤Y5 | | | | | | | to Reference |
| | | | | | | | | Material |
| 1 | ≤BY5, | 5.7 | 4 | 62 | 23 | 91.2 | 5.2 | No new peak > |
| | ≤B5, | | | | | | | 1.0% compared |
| | ≤Y5 | | | | | | | to Reference |
| | | | | | | | | Material |
| 3 | ≤BY5, | 5.6 | 5 | 55 | 11 | 86.4 | 5.1 | No new peak > |
| | ≤B5, | | | | | | | 1.0% compared |
| | ≤Y5 | | | | | | | to Reference |
| | | | | | | | | Material |

| | | | SSE HPLC | | | |
|---|---|---|---|---|---|---|
| | cSD | | Main | | | |
| | (Non-reduced) | | Component | | | Protein Conc. by |
| Months | Purity (%) | New Peaks (%) | (%) | HMWS (%) | LMWS (%) | A₂₈₀ (mg/mL) |
| 0 | 98.2 | No new peak > 1.0% compared | 98.8 | 1.2 | <0.1 | 162 |
| | | to Reference Material | | | | |
| 1 | 94.8 | No new peak > 1.0% compared | 97.0 | 2.5 | 0.5 | 161 |
| | | to Reference Material | | | | |
| 3 | 88.5 | No new peak > 1.0% compared | 92.6 | 5.8 | 1.6 | 162 |
| | | to Reference Material | | | | |

| | | | | EGFR | cMET | |
|---|---|---|---|---|---|---|
| | | | | Binding | Binding | |
| | | | | activity | activity | |
| | cIEF | | | relative to | relative to | Polysorbate |
| | Main | Sum of acidic | Sum of Basic | Reference | Reference | 80 |
| Months | peak (%) | peaks (%) | peaks (%) | Material (%) | Material (%) | (%) |
| 0 | 75 | 23 | 3 | 89 | 85 | 0.06 |
| 1 | 43 | 51 | 6 | 39 | 84 | 0.06 |
| 3 | 15 | 81 | 4 | 8 | 78 | 0.06 |

| | Post Translational Modification-Oxidation Site | | | | | |
|---|---|---|---|---|---|---|
| | anti-EGFR | anti-EGFR | anti-EGFR | anti-EGFR | anti-EGFR | anti-EGFR |
| | HC Met | HCMet | HCMet | HC Met 260/ | HC Met 436/ | n/a/anti-c- |
| | 34*/anti- | 103*/anti- | 108*/anti- | anti-c-Met | anti-c-Met | Met LC Trp |
| | c-Met n/a | c-Met n/a | c-Met n/a | HC Met 254 | HC Met 430 | 32*, Trp 35 |
| Months | (%) | (%) | (%) | (%) | (%) | (%) |
| 0 | 0.3 | 1.7 | 1.8 | 2.8 | 1.1 | 1.1 |
| 1 | 0.7 | 2.8 | 3.0 | 3.9 | 1.8 | 2.2 |
| 3 | 0.4 | 3.7 | 4.8 | 4.1 | 1.7 | 5.2 |

| | Post Translational Modification-Deamidation Site | | |
|---|---|---|---|
| | anti-EGFR HC Asn 333*/ | anti-EGFR HC Asn 392/anti-c-Met | anti-EGFR n/a/anti-c-Met |
| Months | anti-c-Met HC Asn 327 (%) | HC Asn 386, Asn 391 (%) | HC Asn 55*, 59* (%) |
| 0 | 0.7 | 1.5 | 4.3 |
| 1 | 15.2 | 3.8 | 7.0 |
| 3 | NT | 5.8 | 11.5 |

NT = Not tested

| | Post Translational Modification-Isomerization Site | |
|---|---|---|
| Months | anti-EGFR HC Asp 53*, Asp 54*/anti-c-Met n/a (%) | anti-EGFR HC Asp 99* anti-c-Met n/a (%) |
| 0 | 0.6 | 1.5 |
| 1 | 3.0 | 6.7 |
| 3 | 6.2 | 21.0 |

### Example 12: Description of Drug Product Stability Study with rHuPH20

This study was conducted to monitor amivantamab with rHuPH20 DP attributes placed on stability under various environmental conditions and lengths of time. Study test articles were prepared by aliquoting Formulated Bulk into 8R vials at a fill volume of 7.1 mL. The vials were stoppered, capped, and crimp sealed.

All studies were to be performed with vials in an inverted orientation.

TABLE 24
Study parameters

| Stability | Storage | Duration |
|---|---|---|
| Classification | condition | (Months) |
| Real-time | 5 ± 3° C. | 36 |
| Accelerated | 25 ± 2° C./60% RH | 6 |
| Stressed | 40 ± 2° C./75% RH | 3 |

### Stability Study Results

The stability results to date for amivantamab with rHuPH20 DP held under recommended, accelerated, and stressed conditions are listed below in Tables 25-27. It is anticipated that at all-time points for DP held at recommended storage conditions, all test parameter result values observed per assay study will exceed the criteria consistent with the most preferred embodiment of the stability when held after storage for about 12 months or more and at a temperature of about 5° C., after storage for about 12 months or more and at a temperature of about 25° C., and/or after storage for about 2 years or more and at a temperature of about 5° C. Similarly, it is anticipated peptide map results will show little to no consequential change over time in the measured percent of post translational modification.

It is anticipated results for amivantamab with rHuPH20 DP held at accelerated and stressed conditions showed the expected rates of degradation for Drug Product exposed to prolonged accelerated and stressed storage conditions. It is also anticipated that DP held at accelerated conditions (25° C.) for 12 months will show results consistent with the preferred embodiment of the stability when held at 2-8° C. for about 2 years or more.

### 5° C. Data

TABLE 25
Stability Results for Amivantamab with rHuPH20 Drug Product Stored at 5° C.

| | | | | Particulate | | | | |
|---|---|---|---|---|---|---|---|---|
| | | | | Matter | | | | |
| | | | | Sub-visible | | | | |
| | | | | ≥10 | | cSDS (Reduced) | | |
| | | | | μm: | ≥25 μm: | | AG | |
| | Color of | | Turbidity | particles | particles | Purity: | HC: | |
| Months | Solution | pH | (NTU) | per vial | per vial | % | % | new peaks |
| 0 | ≤BY5, | 5.7 | 4.5 | 13 | 0 | 94.5 | 4.9 | No new peak > 1.0% |
| | ≤B5, | | | | | | | compared to Reference |
| | ≤Y5 | | | | | | | Material |
| 3 | ≤BY6, | 5.6 | 4.3 | 26 | 6 | 94.2 | 5.0 | No new peak > 1.0% |
| | ≤B6, | | | | | | | compared to Reference |
| | ≤Y5 | | | | | | | Material |
| 5 | ≤BY6, | 5.6 | 4 | 18 | 2 | 94.1 | 5.0 | No new peak > 1.0% |
| | ≤B6, | | | | | | | compared to Reference |
| | ≤Y5 | | | | | | | Material |
| 6 | ≤BY6, | 5.7 | 4 | 17 | 1 | 94.0 | 5.1 | No new peak > 1.0% |
| | ≤B6, | | | | | | | compared to Reference |
| | ≤Y5 | | | | | | | Material |
| 9 | ≤BY6, | 5.7 | 4 | 61 | 15 | 94.1 | 5.0 | No new peak > 1.0% |
| | ≤B6, | | | | | | | compared to Reference |
| | ≤Y5 | | | | | | | Material |
| 12 | ≤BY5, | 5.7 | 4 | 29 | 0 | 93.8 | 5.0 | No new peak > 1.0% |
| | ≤B6, | | | | | | | compared to Reference |
| | ≤Y5 | | | | | | | Material |

Stability Results for Amivantamab with rHuPH20 Drug Product Stored at 5° C.

| | cSDS (Non-reduced) | | SE HPLC | | | Protein | |
|---|---|---|---|---|---|---|---|
| | | New | Main | | | Conc. by | |
| | Purity | Peaks | Component | HMWS | LMWS | A₂₈₀ | rHuPH20 |
| Months | (%) | (%) | (%) | (%) | (%) | (mg/mL) | (U/mL) |
| 0 | 98.3 | No new | 98.8 | 1.2 | <0.1 | 162 | 2362 |
| | | peak > | | | | | |
| | | 1.0% | | | | | |
| | | compared | | | | | |
| | | to | | | | | |
| | | Reference | | | | | |
| | | Material | | | | | |
| 3 | 98.4 | No new | 98.6 | 1.3 | <0.1 | 162 | 2150 |
| | | peak > | | | | | |
| | | 1.0% | | | | | |
| | | compared | | | | | |
| | | to | | | | | |
| | | Reference | | | | | |
| | | Material | | | | | |
| 5 | 98.2 | No new | 98.6 | 1.4 | <0.1 | 161 | |
| | | peak > | | | | | |
| | | 1.0% | | | | | |
| | | compared | | | | | |
| | | to | | | | | |
| | | Reference | | | | | |
| | | Material | | | | | |
| 6 | 98.1 | No new | 98.5 | 1.4 | <0.10 | 163 | 2323 |
| | | peak > | | | | | |
| | | 1.0% | | | | | |
| | | compared | | | | | |
| | | to | | | | | |
| | | Reference | | | | | |
| | | Material | | | | | |
| 9 | 98.2 | No new | 98.4 | 1.5 | <0.10 | 163 | 2042 |
| | | peak > | | | | | |
| | | 1.0% | | | | | |
| | | compared | | | | | |
| | | to | | | | | |
| | | Reference | | | | | |
| | | Material | | | | | |
| 12 | 98.2 | No new | 98.5 | 1.5 | <0.10 | 162 | 2136 |
| | | peak > | | | | | |
| | | 1.0% | | | | | |
| | | compared | | | | | |
| | | to | | | | | |
| | | Reference | | | | | |
| | | Material | | | | | |

Stability Results for Amivantamab with rHuPH20 Drug Product Stored at 5° C.

| | | | | EGFR | cMET | |
|---|---|---|---|---|---|---|
| | | | | Binding | Binding | |
| | cIEF | | | activity | activity | |
| | Main | Sum of | Sum of | relative to | relative to | |
| | peak | acidic | Basic | Reference | Reference | Polysorbate |
| Months | (%) | peaks (%) | peaks (%) | Material (%) | Material (%) | 80 (%) |
| 0 | 75 | 22 | 3 | 92 | 91 | 0.06 |
| 3 | 74 | 23 | 3 | 82 | 97 | 0.06 |
| 5 | 74 | 23 | 3 | | 100 | 0.06 |
| 6 | 73 | 24 | 3 | 89 | 102 | 0.06 |
| 9 | 73 | 24 | 3 | 87 | 106 | 0.05 |
| 12 | 72 | 25 | 3 | 94 | 97 | 0.05 |

Stability Results for Amivantamab with rHuPH20 Drug Product Stored at 5° C.

| | Post Translational Modification-Oxidation Site | | | | | |
|---|---|---|---|---|---|---|
| | anti-EGFR | anti-EGFR | anti-EGFR | anti-EGFR | anti-EGFR | anti-EGFR |
| | HCMet | HCMet | HCMet | HC Met 260/ | HC Met 436/ | n/a/anti-c- |
| | 34*/anti-c- | 103*/anti- | 108*/anti- | anti-c-Met | anti-c-Met | Met LC Trp |
| | Met n/a | c-Met n/a | c-Met n/a | HC Met 254 | HC Met 430 | 32*, Trp35 |
| Months | (%) | (%) | (%) | (%) | (%) | (%) |
| 0 | 0.4 | 1.8 | 2.2 | 2.9 | 1.2 | 1.0 |
| 3 | 0.4 | 1.5 | 1.6 | 2.4 | 0.9 | 0.9 |
| 5 | 0.2 | 1.5 | 1.8 | 2.4 | 1.0 | 1.1 |
| 6 | 0.2 | 1.3 | 2.8 | 2.8 | 0.6 | 0.7 |
| 9 | 0.4 | 1.5 | 1.9 | 2.5 | 1.0 | 1.2 |
| 12 | 0.1 | 1.1 | 1.4 | 2.1 | 0.7 | 0.9 |

Stability Results for Amivantamab with rHuPH20 Drug Product Store at 5

[symbol]

| | Post Translational Modification-Deamidation Site | | |
|---|---|---|---|
| | anti-EGFR HC Asn | anti-EGFR HC Asn 392/ | |
| | 333*/anti-c-Met HC | anti-c-Met HC Asn 386, | anti-EGFR n/a/ |
| | Asn 327 | Asn 391 | anti-c-Met HC Asn 55*, 59* |
| Months | (%) | (%) | (%) |
| 0 | 0.7 | 1.5 | 4.0 |
| 3 | 0.6 | 1.1 | 3.6 |
| 5 | NT | 2.8 | 4.2 |
| 6 | NT | 1.9 | 3.8 |
| 9 | NT | 2.9 | 4.8 |
| 12 | NT | 1.9 | 3.4 |

Stability Results for Amivantamab with rHuPH20 Drug Product Stored at 5° C.

| | Post Translational Modification-Isomerization Site | |
|---|---|---|
| | anti-EGFR HC | |
| | Asp 53*, Asp | anti-EGFR HC |
| | 54*/anti-c-Met | Asp 99*/anti-c- |
| | n/a | Met n/a |
| Months | (%) | (%) |
| 0 | 0.5 | 1.4 |
| 3 | 0.5 | 1.1 |
| 5 | 0.5 | 1.2 |
| 6 | 0.4 | 1.0 |
| 9 | 0.5 | 1.3 |
| 12 | 0.5 | 1.2 |

NT = Not tested [symbol]
indicates data missing or illegible when filed

### 25° C. Data

TABLE 26
Stability Results for Amivantamab with rHuPH20 Drug Product Stored at 25° C.

| | | | | Particulate Matter | | | | |
|---|---|---|---|---|---|---|---|---|
| | | | | Sub-visible | | cSDS (Reduced) | | |
| | | | | ≥10 μm: | ≥25 μm: | | AG | |
| | Color of | | Turbidity | particles | particles | Purity: | HC: | |
| Months | Solution | pH | (NTU) | per vial | per vial | % | % | new peaks |
| 0 | ≤BY5, | 5.7 | 4.5 | 13 | 0 | 94.5 | 4.9 | No new peak > |
| | ≤B5, ≤Y5 | | | | | | | 1.0% |
| | | | | | | | | compared to |
| | | | | | | | | Reference |
| | | | | | | | | Material |
| 3 | ≤BY6, | 5.6 | 4 | 37 | 2 | 93.2 | 5.0 | No new peak > |
| | ≤B5, ≤Y5 | | | | | | | 1.0% |
| | | | | | | | | compared to |
| | | | | | | | | Reference |
| | | | | | | | | Material |
| 6 | ≤BY6, | 5.6 | 5 | 19 | 0 | 92.4 | 5.2 | No new peak > |
| | ≤B5, ≤Y5 | | | | | | | 1.0% |
| | | | | | | | | compared to |
| | | | | | | | | Reference |
| | | | | | | | | Material |

Stability Results for Amivantamab with rHuPH20 Drug Product Stored at 25□C.

| | cSDS (Non-reduced) | | SE HPLC | | | Protein | |
|---|---|---|---|---|---|---|---|
| | | New | Main | | | Conc. by | |
| | Purity | Peaks | Component | HMWS | LMWS | A₂₈₀ | rHuPH20 |
| Months | (%) | (%) | (%) | (%) | (%) | (mg/mL) | (U/mL) |
| 0 | 98.3 | No new | 98.8 | 1.2 | <0.1 | 162 | 2363 |
| | | peak > | | | | | |
| | | 1.0% | | | | | |
| | | compared | | | | | |
| | | to | | | | | |
| | | Reference | | | | | |
| | | Material | | | | | |
| 3 | 96.8 | No new | 97.8 | 1.9 | 0.2 | 161 | 2318 |
| | | peak > | | | | | |
| | | 1.0% | | | | | |
| | | compared | | | | | |
| | | to | | | | | |
| | | Reference | | | | | |
| | | Material | | | | | |
| 6 | 95.9 | No new | 97.3 | 2.2 | 0.6 | 163 | 2040 |
| | | peak > | | | | | |
| | | 1.0% | | | | | |
| | | compared | | | | | |
| | | to | | | | | |
| | | Reference | | | | | |
| | | Material | | | | | |

Stability Results for Amivantamab with rHuPH20 Drug Product Stored at 25° C.

| | cIEF | | | EGFR Binding | cMET Binding | |
|---|---|---|---|---|---|---|
| | Main | Sum of | Sum of | activity relative | activity relative | |
| | peak | acidic | Basic | to Reference | to Reference | Polysorbate |
| Months | (%) | peaks (%) | peaks (%) | Material (%) | Material (%) | 80 (%) |
| 0 | 75 | 22 | 3 | 92 | 91 | 0.06 |
| 3 | 60 | 35 | 5 | | 81 | 0.06 |
| 6 | 53 | 42 | 5 | 54 | 103 | 0.06 |

Stability Results for Amivantamab with rHuPH20 Drug Product Stored at 25° C.

| | Post Translational Modification-Oxidation Site | | | | | |
|---|---|---|---|---|---|---|
| | anti-EGFR | anti-EGFR | anti-EGFR | anti-EGFR | anti-EGFR | anti-EGFR |
| | HC Met | HCMet | HCMet | HC Met 260/ | HC Met 436/ | n/a/anti-c- |
| | 34*/anti-c- | 103*/anti- | 108*/anti- | anti-c-Met | anti-c-Met | Met LC Trp |
| | Met n/a | c-Met n/a | c-Met n/a | HC Met 254 | HC Met 430 | 32*, Trp 35 |
| Months | (%) | (%) | (%) | (%) | (%) | (%) |
| 0 | 0.4 | 1.8 | 2.2 | 2.9 | 1.2 | 1.0 |
| 3 | 0.4 | 2.6 | 3.4 | 3.5 | 1.4 | 2.0 |
| 6 | 0.1 | 1.7 | 3.2 | 3.0 | 0.7 | 1.5 |

Stability Results for Amivantamab with rHuPH20 Drug Product Stored at 25° C.

| | Post Translational Modification-Deamidation Site | | |
|---|---|---|---|
| | anti-EGFR HC | anti-EGFR HC | |
| | Asn 333*/anti- | Asn 392/anti-c- | anti-EGFR n/a/ |
| | c-Met HC Asn | Met HC Asn | anti-c-Met HC |
| | 327 | 386, Asn 391 | Asn 55*, 59* |
| Months | (%) | (%) | (%) |
| 0 | 0.7 | 1.5 | 4.0 |
| 3 | NT | 2.7 | 4.8 |
| 6 | NT | 2.7 | 5.7 |

Stability Results for Amivantamab with rHuPH20 Drug Product Stored at 25° C.

| | Post Translational Modification-Isomerization Site | |
|---|---|---|
| | anti-EGFR HC Asp 53*, | anti-EGFR HC Asp |
| | Asp 54*/anti-c-Metn/a | 99*/anti-c-Metn/a |
| Months | (%) | (%) |
| 0 | 0.5 | 1.4 |
| 3 | 1.2 | 3.5 |
| 6 | 2.1 | 4.6 |

NT = Not tested

### 40° C. Data

TABLE 27
Stability Results for Amivantamab with rHuPH20 Drug Product Stored at 40° C.

| | | | | Particulate Matter | | | | |
|---|---|---|---|---|---|---|---|---|
| | | | | Sub-visible | | | | |
| | | | | ≥10 | ≥25 | cSDS (Reduced) | | |
| | | | | μm: | μm: | | AG | |
| | Color of | | Turbidity | particles | particles | Purity: | HC: | |
| Months | Solution | pH | (NTU) | per vial | per vial | % | % | new peaks |
| 0 | ≤BY5, | 5.7 | 4.5 | 13 | 0 | 94.5 | 4.9 | No new peak > |
| | ≤B5, ≤Y5 | | | | | | | 1.0% |
| | | | | | | | | compared to |
| | | | | | | | | Reference |
| | | | | | | | | Material |
| 1 | ≤BY5, | 5.7 | 4.6 | 34 | 2 | 90.9 | 5.2 | No new peak > |
| | ≤B5, ≤Y5 | | | | | | | 1.0% |
| | | | | | | | | compared to |
| | | | | | | | | Reference |
| | | | | | | | | Material |
| 3 | ≤BY5, | 5.6 | 5 | 105 | 65 | 86.8 | 5.0 | No new peak > |
| | ≤B5, ≤Y5 | | | | | | | 1.0% |
| | | | | | | | | compared to |
| | | | | | | | | Reference |
| | | | | | | | | Material |

| | | | SE HPLC | | | Protein | |
|---|---|---|---|---|---|---|---|
| | cSDS(Non-reduced) | | Main | | | Conc. by | |
| | Purity | New | Component | HMWS | LMWS | A₂₈₀ | rHuPH20 |
| Months | (%) | Peaks | (%) | (%) | (%) | (mg/mL) | (U/mL) |
| 0 | 98.3 | No new | 98.9 | <0.1 | <0.1 | 162 | 2150 |
| | | peak > | | | | | |
| | | 1.0% | | | | | |
| | | compared | | | | | |
| | | to | | | | | |
| | | Reference | | | | | |
| | | Material | | | | | |
| 1 | 94.9 | No new | 96.9 | 2.6 | 0.5 | 162 | <215 |
| | | peak > | | | | | |
| | | 1.0% | | | | | |
| | | compared | | | | | |
| | | to | | | | | |
| | | Reference | | | | | |
| | | Material | | | | | |
| 3 | 88.7 | No new | 93.0 | 5.5 | 1.5 | 163 | <215 |
| | | peak > | | | | | |
| | | 1.0% | | | | | |
| | | compared | | | | | |
| | | to | | | | | |
| | | Reference | | | | | |
| | | Material | | | | | |

| | cIEF | | | EGFR Binding | cMET Binding | |
|---|---|---|---|---|---|---|
| | | Sum of | Sum of | activity relative | activity relative | |
| | Main | acidic | Basic | to Reference | to Reference | Polysorbate |
| Months | peak (%) | peaks (%) | peaks(%) | Material (%) | Material (%) | 80 (%) |
| 0 | 75 | 22 | 3 | 92 | 91 | 0.06 |
| 1 | 42 | 52 | 6 | 41 | 81 | 0.06 |
| 3 | 16 | 79 | 4 | 9 | 68 | 0.06 |
| | Post Translational Modification-Oxidation Site | | | | | |
| | anti-EGFR | anti-EGFR | anti-EGFR | anti-EGFR | anti-EGFR | anti-EGFR |
| | HC Met | HCMet | HCMet | HC Met 260/ | HC Met 436/ | n/a/anti-c- |
| | 34*/anti-c- | 103*/anti- | 108*/anti- | anti-c-Met | anti-c-Met | Met LC Trp |
| | Met n/a | c-Met n/a | c-Met n/a | HC Met 254 | HC Met 430 | 32*, Trp 35 |
| Months | (%) | (%) | (%) | (%) | (%) | (%) |
| 0 | 0.4 | 1.8 | 2.2 | 2.9 | 1.2 | 1.0 |
| 1 | 0.9 | 3.0 | 3.1 | 3.8 | 1.9 | 2.2 |
| 3 | 0.4 | 3.5 | 4.7 | 4.2 | 1.7 | 3.8 |

| | Post Translational Modification-Deamidation Site | | |
|---|---|---|---|
| | anti-EGFR HC | anti-EGFR HC | |
| | Asn 333*/anti- | Asn 392/anti-c- | anti-EGFR n/a/ |
| | c-Met HC Asn | Met HC Asn | anti-c-Met HC |
| | 327 | 386, Asn 391 | Asn 55*, 59* |
| Months | (%) | (%) | (%) |
| 0 | 0.7 | 1.5 | 4.0 |
| 1 | 15.4 | 3.8 | 7.1 |
| 3 | NT | 5.6 | 11.0 |

| | Post Translational Modification-Isomerization Site | |
|---|---|---|
| | anti-EGFR HC Asp 53*, | anti-EGFR HC Asp |
| | Asp 54*/anti-c-Metn/a | 99*/anti-c-Metn/a |
| Months | (%) | (%) |
| 0 | 1.5 | 4.0 |
| 1 | 3.8 | 7.1 |
| 3 | 5.6 | 11.0 |

NT = Not tested

### Example 12A: Viscosity

The viscosity of 160 mg/mL amivantamab, 30 mM Acetate, 8.5% Sucrose, 0.06% polysorbate 80, 20 g/mL EDTA, 1 mg/mL methionine at pH 5.7 was analyzed with an automated viscometer. A temperature sweep was performed between 4° C. and 40° C. at 5° C. intervals with four measurements per temperature setting. The first measurement at all temperatures was discarded and the remaining three are used to calculate the average viscosity value reported in the Table 28 below.

TABLE 28
Viscosity

| | Avg. |
|---|---|
| Temperature | Viscosity |
| (° C.) | (cP) |
| 4.0 | 21.9 |
| 10.0 | 16.0 |
| 15.0 | 13.3 |
| 20.0 | 11.0 |
| 25.0 | 9.3 |
| 30.0 | 7.9 |
| 35.0 | 6.7 |
| 40.0 | 5.8 |

These results demonstrate a high concentration formulation of amivantamab have viscosities that are suitable for injection.

### Example 13. Subcutaneous Delivery of Amivantamab in Patients with Advanced Solid Malignancies

The subcutaneous (SC) delivery of amivantamab was evaluated in a phase 1 dose escalation study in patients with advanced solid tumors who may derive benefit from EGFR or MET-directed therapy (PALOMA; NCT04606381). Eligible tumor types included non-small cell lung cancer (NSCLC), squamous cell carcinoma of the head and neck (SCCHN), hepatocellular cancer (HCC), colorectal cancer (CRC), renal cell cancer (RCC), medullary thyroid cancer (MTC), gastroesophageal cancer (GEC), mesothelioma, breast cancer (BC) and ovarian cancer (OC). Eligible patients have progressed after standard of care therapy for metastatic disease, were ineligible for, or have declined current standard therapies.

The study objectives were to evaluate the feasibility of administration, safety, and pharmacokinetics (PK) of a low concentration formulation, 50 mg/mL of amivantamab ±rHuPH20 (Part 1) and a high concentration formulation, 160 mg/mL of amivantamab ±rHuPH20 (Part 2). The low concentration formulation (50 mg/mL) amivantamab was administered either with (Ami-LC-MD [mix and deliver]) or without (Ami-LC) rHuPH20 (Part 1, Cohorts 1a and 1b, respectively). The high concentration formulation (160 mg/mL) amivantamab was administered either with (Ami-HC-CF [coformulated]) or without (Ami-HC) rHuPH20 (Part 2, Cohorts 2a and 2b, respectively). Patients in Part 1 and Part 2 received a dosage of amivantamab at 1050 mg and 2,000 Units/mL rHuPH20 (1400 mg and 2000 Units/mL rHuPH20 for bodyweight≥80 kg) SC (weekly for the first 4 weeks and every other week thereafter). This study also evaluated administering the full dose of amivantamab on the first day.

Results: The full safety, PK, bioavailability, and receptor occupancy data of patients enrolled in Part 1 (n=16) and Part 2 (n=17) were evaluated. Compared to IV administration, initial SC experience demonstrated that the co-formulation of high concentration amivantamab with rHuPH20 shortened the needed infusion time from 2-4 hours to less than 5 minutes, with initial bioavailability of approximately 65% of IV administration. Saturation of soluble free EGFR and MET was achieved after the first SC dose. The incidence of IRRs was 18.2%, with all events of grade 1-2 severity as compared to 67.3% of patients who received the recommended phase 2 dose (RP2D) of IV amivantamab across the CHRYSALIS study (Park Ann Oncol 32[suppl_5]:S981). The full amivantamab SC dose was safely given at first administration to 14 patients, obviating the need for split dosing.

No increased risk of IRR was observed with the full initial amivantamab dosing; IRR was reported in 3/14 (21%) patients who received the full dose (C1D1) and 3/19 (16%) with the split dose.

The AE profile, outside of IRRs, of SC amivantamab was comparable to IV amivantamab. Grade≥3 AEs occurred in 27.3% of patients, with most reported as single events. A single grade 3 event (hypokalemia) was reported as treatment-related. No treatment-related AEs leading to dose reductions or discontinuations were reported. Two patients (6.1%) had grade 1 injection-site reactions, which were transient and did not impact subsequent dosing.

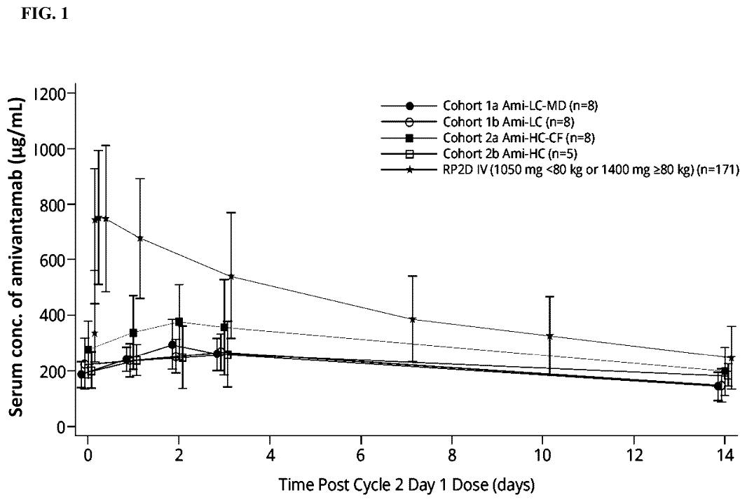

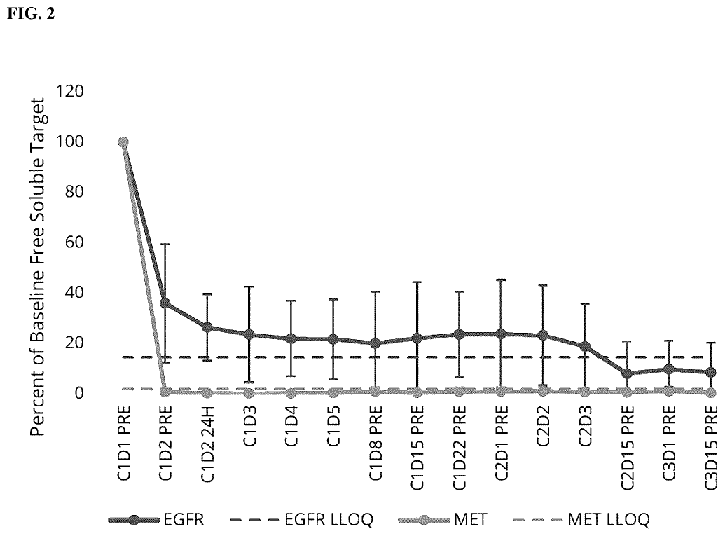

Pharmacokinetics, pharmacodynamics, and immunogenicity. Maximum serum concentration of amivantamab was achieved 2-3 days following SC administration (FIG. 1). Higher exposure was obtained with rHuPH20; the estimated bioavailability with SC administration using formulations with rHuPH20 was approximately 65%. Saturation of soluble free EGFR and MET was achieved in all cohorts after the first full dose (FIG. 2). No anti-drug antibodies were detected in the patients tested (N=33 total in both Part 1 and Part 2 groups).

Conclusions: Initial SC amivantamab ±rHuPH20 was well tolerated with improvements in time and ease of administration and associated with a meaningful reduction in IRRs compared with intravenous (IV) administration, eliminating the need for split dosing.

### Example 14. Amivantamab and Lazertinib in Patients with EGFR-Mutant Non-Small Cell Lung (NSCLC) after Progression on Osimertinib and Platinum-Based Chemotherapy

Methods: A clinical trial cohort evaluated amivantamab and lazertinib in patients with EGFR exon 19 deletion or L858R NSCLC whose disease progressed on 1^{st}/2^{nd}-line osimertinib followed by platinum-chemo as last line of therapy (target population, n=106) and among a more heavily pretreated population (n=56) whose disease progressed after osimertinib and platinum-chemo ±other therapies without regard to number and sequence of these therapies. Patients received 1050 mg IV amivantamab (1400 mg, ≥80 kg)+240 mg oral lazertinib. Response was assessed per RECIST v1.1 (European Journal of Cancer, vol. 45, pp. 228-247 (2009)) and is reported for efficacy-evaluable patients, defined as those who had a ≥6 mo of follow-up for response durability.

Results: 162 patients were enrolled in the cohort (median 62 y, 65% women, 61% Asian, median of 3 [range, 2-14] prior lines of therapy). Median time between last osimertinib treatment to first dose of amivantamab+lazertinib was 6.3 mo and 2.0 mo for the target and heavily pretreated populations, respectively. Of 50 efficacy-evaluable patients in the target population, the ORR was 36% (95% CI, 23-51), with 1 complete response (CR) and 17 partial responses (PRs), and the clinical benefit rate (CBR) was 58% (95% CI, 43-72). Median duration of response (mDOR) was not reached. At a median follow-up of 8.3 mo, 7 responders (39%) have achieved a DOR lasting ≥6 mo. Of 56 efficacy-evaluable patients in the heavily-pretreated population (8.7-mo median follow-up), ORR was 29% (95% CI, 17-42), with 1 CR and 15 PRs. CBR was 55% (95% CI, 42-69) and mDOR was 8.6 mo (95% CI, 4.2-NR). Preliminary evidence of CNS antitumor activity was reported among 8 patients with baseline brain lesions (7 non-target, 1 target) who had not received radiation within 1 year prior to study enrollment.

Infusion-related reaction (TRR) (65%) was the most frequent adverse event (AE) reported, followed by paronychia (49%), rash (41%), and stomatitis (39%). Most common grade ≥3 treatment-related AEs (TRAEs) were infusion-related reactions (7%), acneiform dermatitis (5%), and hypoalbuminemia (4%). TRAEs leading to discontinuation of either or both ami and laz occurred in 12% and 7%, respectively.

### Example 15. Subcutaneous Delivery of Amivantamab, Co-Formulated with rHuPH20

The proposed preliminary recommended phase 2 dose (RP2D) for once every two weeks (Q2W) amivantamab dosing regimens is 1600 mg for patients with a BW<80 kg and 2240 mg for participants with a BW≥80 kg. This preliminary subcutaneous dose comprises amivantamab at the concentration of 160 mg/mL co-formulated with rHuPH20 (amivantamab SC-CF).

The proposed recommended dosing regimens for amivantamab SC-CF are selected to ensure that the resulting exposures are similar to those observed with the IV RP2D regimens. Bioavailability was estimated by comparing the observed AUC following SC dosing (cohorts with and without rHuPH20 formulations) to the corresponding observed AUC following IV dosing. The SC cohorts were dosed at the IV RP2D (1050 mg for participants with a BW<80 kg and 1400 mg for participants with a BW≥80 kg). At this dose, saturation of soluble free EGFR and cMet was achieved after the first SC dose. Moreover, all 33 participants were negative for anti-amivantamab antibodies. Following the first dose in Cycle 2 (after the weekly induction dosing in Cycle 1), the mean (CV %, n) area under the concentration time curves (AUC)C2D1-C2D15 were 95,416 μg×h/mL (45.4%, 7) and 75,378 μg×h/mL (27.0%, 5) for cohorts from 160 mg/ml with and without rHuPH20, respectively.

Co formulation of amivantamab with rHuPH20 (ie, amivantamab SC-CF) provided improved bioavailability (65% vs 51%), compared to formulations without rHuPH20, in addition to shortening the needed for injection time by approximately eight-fold. Based on the estimated bioavailability, a preliminary RP2D of 1,600 mg for participants with a BW<80 kg and 2,240 mg for participants with a BW≥80 kg was proposed.

Emerging data indicates a lower incidence of IRRs (18.7% overall and 0 Grade≥3) than previously reported with amivantamab IV (65.9% overall and 2.3% Grade≥3). As a result of the reduced incidence and severity of IRRs, the study demonstrated the feasibility of a single day infusion of amivantamab SC for the first dose.

Based on the current clinical data and analysis supported by preliminary modeling, amivantamab SC-CF will be administered at the preliminary RP2D of 1,600 mg for participants <80 kg body weight and 2,240 mg for participants >80 kg body weight.

Cohort 1 will assess the combination of amivantamab SC-CF (Q2W) and lazertinib in subjects with treatment-naïve locally advanced or metastatic NSCLC harboring an EGFR exon 19del or exon 21 L858R mutation. Participants will receive amivantamab on Cycle 1 Days 1, 8, 15, and 22 and on Day 1 and 15 of each subsequent 28-day cycle, starting with Cycle 2. Amivantamab SC-CF will be administered SC by manual injection at 1,600 mg (2,240 mg if body weight≥80 kg). Lazertinib will be given 240 mg orally once daily.

Cohort 4 will assess the feasibility of amivantamab SC-CF (Q2W) as a switch therapy in subjects who received ≥3 months of amivantamab IV as per standard of care. Participants will receive amivantamab on Cycle 1 Days 1, 8, 15, and 22 and on Day 1 and 15 of each subsequent 28-day cycle, starting with Cycle 2. Amivantamab SC-CF will be administered by manual injection at 1,600 mg (2,240 mg if body weight≥80 kg).

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

TABLE 29
Sequences

| SEQ ID NO: | Sequence |
|---|---|
| SEQ ID NO: 1 | TYGMH |
| HCDR1, EGFR binding | |
| arm (Kabat method) | |
| SEQ ID NO: 2 | VIWDDGSYKYYGDSVKG |
| HCDR2, EGFR binding | |
| arm (Kabat method) | |
| SEQ ID NO: 3 | DGITMVRGVMKDYFDY |
| HCDR3, EGFR binding | |
| arm (Kabat method) | |
| SEQ ID NO: 4 LCDR1, | RASQDISSALV |
| EGFR binding arm (Kabat | |
| method) | |
| SEQ ID NO: 5 LCDR2, | DASSLES |
| EGFR binding arm (Kabat | |
| method) | |
| SEQ ID NO: 6 LCDR3, | QQFNSYPLT |
| EGFR binding arm (Kabat | |
| method) | |
| SEQ ID NO: 7 HCDR1, c- | SYGIS |
| Met binding arm (Kabat | |
| method) | |
| SEQ ID NO: 8 HCDR2, c- | WISAYNGYTNYAQKLQG |
| Met binding arm (Kabat | |
| method) | |
| SEQ ID NO: 9 HCDR3, c- | DLRGTNYFDY |
| Met binding arm (Kabat | |
| method) | |
| SEQ ID NO: 10 LCDR1, c- | RASQGISNWLA |
| Met binding arm (Kabat | |
| method) | |
| SEQ ID NO: 11 LCDR2, c- | AASSLLS |
| Met binding arm (Kabat | |
| method) | |
| SEQ ID NO: 12 LCDR3, c- | QQANSFPIT |
| Met binding arm (Kabat | |
| method). | |
| SEQ ID NO: 13 | QVQLVESGGGVVQPGRSLRLSCAASGFTFSTYGMHWVRQAPGKGL |
| VH1, EGFR binding arm | WVAVIWDDGSYKYYGDSVKGRFTISRDNSKNTLYL |
| | QMNSLRAEDTAVYYCARDGITMVRGVMKDYFDYWGQGTLVTVSS |
| SEQ ID NO: 14 | AIQLTQSPSSLSASVGDRVTITCRASQDISSALVWYQQKPGKAPKLLI |
| VL1, EGFR binding arm | DASSLESGVPSRFSGSESGTDFTLTISSLQPEDFATYY |
| | CQQFNSYPLTFGGGTKVEIK |
| SEQ ID NO: 15 | QVQLVQSGAEVKKPGASVKVSCETSGYTFTSYGISWVRQAPGHGLE |
| VH2, c-Met binding arm | WMGWISAYNGYTNYAQKLQGRVTMTTDTSTSTAYM |
| | ELRSLRSDDTAVYYCARDLRGTNYFDYWGQGTLVTVSS |
| SEQ ID NO: 16 | DIQMTQSPSSVSASVGDRVTITCRASQGISNWLAWFQHKPGKAPKLI |
| VL2, c-Met binding arm | YAASSLLSGVPSRFSGSGSGTDFTLTISSLQPEDFATYY |
| | CQQANSFPITFGQGTRLEIK |
| SEQ ID NO: 17 HC1 | QVQLVESGGGVVQPGRSLRLSCAASGFTFSTYGMHWVRQAPGKG |
| | LEWVAVIWDDGSYKYYGDSVKGRFTISRDNSKNTLYLQMNSLRA |
| | EDTAVYYCARDGITMVRGVMKDYFDYWGQGTLVTVSSASTKGP |
| | SVFPLAPSSKSTSGGTAALGCLVKDYFPEPVTVSWNSGALTSGVHT |
| | FPAVLQSSGLYSLSSVVTVPSSSLGTQTYICNVNHKPSNTKVDKRV |
| | EPKSCDKTHTCPPCPAPELLGGPSVFLFPPKPKDTLMISRTPEVTCV |
| | VVDVSHEDPEVKFNWYVDGVEVHNAKTKPREEQYNSTYRVVSV |
| | LTVLHQDWLNGKEYKCKVSNKALPAPIEKTISKAKGQPREPQVYT |
| | LPPSREEMTKNQVSLTCLVKGFYPSDIAVEWESNGQPENNYKTTPP |
| | VLDSDGSFLLYSKLTVDKSRWQQGNVFSCSVMHEALHNHYTQKS |
| | LSLSPGK |
| SEQ ID NO: 18 LC1 | AIQLTQSPSSLSASVGDRVTITCRASQDISSALVWYQQKPGKAPKL |
| | LIYDASSLESGVPSRFSGSESGTDFTLTISSLQPEDFATYYCQQFNSY |
| | PLTFGGGTKVEIKRTVAAPSVFIFPPSDEQLKSGTASVVCLLNNFYP |
| | REAKVQWKVDNALQSGNSQESVTEQDSKDSTYSLSSTLTLSKADY |
| | EKHKVYACEVTHQGLSSPVTKSFNRGEC |
| SEQ ID NO: 19 HC2 | QVQLVQSGAEVKKPGASVKVSCETSGYTFTSYGISWVRQAPGHGL |
| | EWMGWISAYNGYTNYAQKLQGRVTMTTDTSTSTAYMELRSLRS |
| | DDTAVYYCARDLRGTNYFDYWGQGTLVTVSSASTKGPSVFPLAP |
| | SSKSTSGGTAALGCLVKDYFPEPVTVSWNSGALTSGVHTFPAVLQ |
| | SSGLYSLSSVVTVPSSSLGTQTYICNVNHKPSNTKVDKRVEPKSCD |
| | KTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQD |
| | WLNGKEYKCKVSNKALPAPIEKTISKAKGQPREPQVYTLPPSREE |
| | MTKNQVSLTCLVKGFYPSDIAVEWESNGQPENNYKTTPPVLDSDG |
| | SFFLYSRLTVDKSRWOOGNVFSCSVMHEALHNHYTOKSLSLSPGK |
| SEQ ID NO: 20 LC2 | DIQMTQSPSSVSASVGDRVTITCRASQGISNWLAWFQHKPGKAPK |
| | LLIYAASSLLSGVPSRFSGSGSGTDFTLTISSLQPEDFATYYCQQAN |
| | SFPITFGQGTRLEIKRTVAAPSVFIFPPSDEQLKSGTASVVCLLNNFY |
| | PREAKVQWKVDNALQSGNSQESVTEQDSKDSTYSLSSTLTLSKAD |
| | YEKHKVYACEVTHQGLSSPVTKSFNRGEC |
| SEQ ID NO: 21: rHuPH20 | LNFRAPPVIPNVPFLWAWNAPSEFCLGKFDEPLDMSLFSFIGSPRIN |
| | ATGQGVTIFYVDRLGYYPYIDSITGVTVNGGIPQKISLQDHLDKAK |
| | KDITFYMPVDNLGMAVIDWEEWRPTWARNWKPKDVYKNRSIEL |
| | VQQQNVQLSLTEATEKAKQEFEKAGKDFLVETIKLGKLLRPNHLW |
| | GYYLFPDCYNHHYKKPGYNGSCFNVEIKRNDDLSWLWNESTALY |
| | PSIYLNTQQSPVAATLYVRNRVREAIRVSKIPDAKSPLPVFAYTRIV |
| | FTDQVLKFLSQDELVYTFGETVALGASGIVIWGTLSIMRSMKSCLL |
| | LDNYMETILNPYIINVTLAAKMCSQVLCQEQGVCIRKNWNSSDYL |
| | HLNPDNFAIQLEKGGKFTVRGKPTLEDLEQFSEKFYCSCYSTLSCK |
| | EKADVKDTDAVDVCIADGVCIDAFLKPPMETEEP |
| SEQ ID NO: 22: | LNFRAPPVIPNVPFLWAWNAPSEFCLGKFDEPLDMSLFSFIGSPRIN |
| rHuPH20 variant 1 | ATGQGVTIFYVDRLGYYPYIDSITGVTVNGGIPQKISLQDHLDKAK |
| | KDITFYMPVDNLGMAVIDWEEWRPTWARNWKPKDVYKNRSIEL |
| | VQQQNVQLSLTEATEKAKQEFEKAGKDFLVETIKLGKLLRPNHLW |
| | GYYLFPDCYNHHYKKPGYNGSCFNVEIKRNDDLSWLWNESTALY |
| | PSIYLNTQQSPVAATLYVRNRVREAIRVSKIPDAKSPLPVFAYTRIV |
| | FTDQVLKFLSQDELVYTFGETVALGASGIVIWGTLSIMRSMKSCLL |
| | LDNYMETILNPYIINVTLAAKMCSQVLCQEQGVCIRKNWNSSDYL |
| | HLNPDNFAIQLEKGGKFTVRGKPTLEDLEQFSEKFYCSCYSTLSCK |
| | EKADVKDTDAVDVCIADGVCIDAFLKPPMETEEPQIFY |
| SEQ ID NO: 23: | LNFRAPPVIPNVPFLWAWNAPSEFCLGKFDEPLDMSLFSFIGSPRIN |
| rHuPH20 variant 2 | ATGQGVTIFYVDRLGYYPYIDSITGVTVNGGIPQKISLQDHLDKAK |
| | KDITFYMPVDNLGMAVIDWEEWRPTWARNWKPKDVYKNRSIEL |
| | VQQQNVQLSLTEATEKAKQEFEKAGKDFLVETIKLGKLLRPNHLW |
| | GYYLFPDCYNHHYKKPGYNGSCFNVEIKRNDDLSWLWNESTALY |
| | PSIYLNTQQSPVAATLYVRNRVREAIRVSKIPDAKSPLPVFAYTRIV |
| | FTDQVLKFLSQDELVYTFGETVALGASGIVIWGTLSIMRSMKSCLL |
| | LDNYMETILNPYIINVTLAAKMCSQVLCQEQGVCIRKNWNSSDYL |
| | HLNPDNFAIQLEKGGKFTVRGKPTLEDLEQFSEKFYCSCYSTLSCK |
| | EKADVKDTDAVDVCIADGVCIDAFLKPPMETEEPQIF |
| SEQ ID NO: 24: | LNFRAPPVIPNVPFLWAWNAPSEFCLGKFDEPLDMSLFSFIGSPRIN |
| rHuPH20 variant 3 | ATGQGVTIFYVDRLGYYPYIDSITGVTVNGGIPQKISLQDHLDKAK |
| | KDITFYMPVDNLGMAVIDWEEWRPTWARNWKPKDVYKNRSIEL |
| | VQQQNVQLSLTEATEKAKQEFEKAGKDFLVETIKLGKLLRPNHLW |
| | GYYLFPDCYNHHYKKPGYNGSCFNVEIKRNDDLSWLWNESTALY |
| | PSIYLNTQQSPVAATLYVRNRVREAIRVSKIPDAKSPLPVFAYTRIV |
| | FTDQVLKFLSQDELVYTFGETVALGASGIVIWGTLSIMRSMKSCLL |
| | LDNYMETILNPYIINVTLAAKMCSQVLCQEQGVCIRKNWNSSDYL |
| | HLNPDNFAIQLEKGGKFTVRGKPTLEDLEQFSEKFYCSCYSTLSCK |
| | EKADVKDTDAVDVCIADGVCIDAFLKPPMETEEPQI |
| SEQ ID NO: 25: | LNFRAPPVIPNVPFLWAWNAPSEFCLGKFDEPLDMSLFSFIGSPRIN |
| rHuPH20 variant 4 | ATGQGVTIFYVDRLGYYPYIDSITGVTVNGGIPQKISLQDHLDKAK |
| | KDITFYMPVDNLGMAVIDWEEWRPTWARNWKPKDVYKNRSIEL |
| | VQQQNVQLSLTEATEKAKQEFEKAGKDFLVETIKLGKLLRPNHLW |
| | GYYLFPDCYNHHYKKPGYNGSCFNVEIKRNDDLSWLWNESTALY |
| | PSIYLNTQQSPVAATLYVRNRVREAIRVSKIPDAKSPLPVFAYTRIV |
| | FTDQVLKFLSQDELVYTFGETVALGASGIVIWGTLSIMRSMKSCLL |
| | LDNYMETILNPYIINVTLAAKMCSQVLCQEQGVCIRKNWNSSDYL |
| | HLNPDNFAIQLEKGGKFTVRGKPTLEDLEQFSEKFYCSCYSTLSCK |
| | EKADVKDTDAVDVCIADGVCIDAFLKPPMETEEPQ |

1. A stable aqueous pharmaceutical composition comprising a bispecific epidermal growth factor receptor (EGFR)/hepatocyte growth factor receptor (c-Met) antibody and a hyaluronidase, wherein the antibody comprises:
a. a first heavy chain (HC1) comprising a HC1 variable region (VH1) comprising a heavy chain complementarity determining region 1 (HCDR1), a HCDR2 and a HCDR3 comprising the amino acid sequences of SEQ ID NOs: 1, 2, and 3, respectively;
b. a first light chain (LC1) comprising a LC1 variable region (VL1) comprising a light chain complementarity determining region 1 (LCDR1), a LCDR2 and a LCDR3 comprising the amino acid sequences of SEQ ID NOs: 4, 5, and 6, respectively;
c. a second heavy chain (HC2) comprising a HC2 variable region (VH2) comprising a HCDR1, a HCDR2 and a HCDR3 comprising the amino acid sequences of SEQ ID NOs: 7, 8, and 9;
d. a second light chain (LC2) comprising a LC2 variable region (VL2) comprising a LCDR1, a LCDR2 and a LCDR3 comprising the amino acid sequences of SEQ ID NOs: 10, 11, and 12, respectively.

2. The stable aqueous pharmaceutical composition of claim 1 comprising:
a) about 144 mg/mL to about 176 mg/mL of a bispecific epidermal growth factor receptor (EGFR)/hepatocyte growth factor receptor (c-Met) antibody,
b) about 10 mM to about 50 mM of acetate and/or pharmaceutically acceptable acetate salt,
c) about 6.8% (w/v) to about 10.2% (w/v) of sucrose,
d) about 0.036% (w/v) to about 0.084% (w/v) of polysorbate 80 (PS80),
e) about to 0.8 mg/mL to about 1.2 mg/mL of methionine,
f) about 16 μg/mL to about 24 μg/mL of ethylenediaminetetraacetic acid (EDTA),
g) optionally, about 1,000 U/mL to about 3,000 U/mL of hyaluronidase; and
h) a pH from about 5.2 to about 6.2.

3. The stable aqueous pharmaceutical composition of claim 1, wherein the bispecific EGFR-cMet antibody comprises a HC1 variable region comprising the amino acid sequence of SEQ ID NO:13; a LC1 variable region comprising the amino acid sequence of SEQ ID NO:14; a HC2 variable region comprising the amino acid sequence of SEQ ID NO:15; and a LC2 variable region comprising the amino acid sequence of SEQ ID NO:16.

4. The stable aqueous pharmaceutical composition of claim 1, wherein the HC1 comprises the amino acid sequence of SEQ ID NO:17; the LC1 comprises the amino acid sequence of SEQ ID NO:18; the HC2 comprises the amino acid sequence of SEQ ID NO: 19; and the LC2 comprises the amino acid sequence of SEQ ID NO:20.

5. The stable aqueous pharmaceutical composition of claim 1, wherein the bispecific EGFR-cMet antibody is amivantamab or a biosimilar thereof.

6. The stable aqueous pharmaceutical composition of claim 2, wherein the bispecific EGFR-cMet antibody has a concentration of about 160 mg/mL.

7. The stable aqueous pharmaceutical composition of claim 2, wherein the acetate and/or pharmaceutically acceptable acetate salt has a concentration of about 30 mM.

8. The stable aqueous pharmaceutical composition of claim 2, wherein the acetate and/or pharmaceutically acceptable acetate salt comprises glacial acetic acid and/or sodium acetate trihydrate.

9. The stable aqueous pharmaceutical composition of claim 2, comprising about 8.5% (w/v) sucrose.

10. The stable aqueous pharmaceutical composition of claim 2, comprising about 0.06% (w/v) PS80.

11. The stable aqueous pharmaceutical composition of claim 2, wherein the methionine comprises L-methionine and has a concentration of about 1 mg/mL.

12. The stable aqueous pharmaceutical composition of claim 2, wherein the EDTA has a concentration of about 20 μg/mL.

13. The stable aqueous pharmaceutical composition of claim 2, wherein the pH is about 5.7.

14. The stable aqueous pharmaceutical composition of claim 2, wherein the hyaluronidase is a human hyaluronidase, optionally a soluble human PH20 comprising the amino acid sequence of SEQ ID NO: 21-25.

15. The stable aqueous pharmaceutical composition of claim 2, wherein the composition comprises rHuPH20 at a concentration of about 1,000 U/mL to about 3,000 U/mL.

16. The stable aqueous pharmaceutical composition of claim 2, wherein the hyaluronidase is a human recombinant hyaluronidase enzyme PH20 (rHuPH20) at a concentration of about 2,000 U/mL.

17. The stable aqueous pharmaceutical composition of claim 2, comprising about 160 mg/mL of the bispecific EGFR-cMet antibody, about 30 mM acetate and/or pharmaceutically acceptable acetate salt, about 8.5% sucrose, and about 1 mg/mL L-methionine with polysorbate 80 to a final concentration of about 0.06% (w/v), and EDTA to a final concentration of about 20 μg/mL, wherein the stable aqueous pharmaceutical composition has pH about 5.7,
and wherein the bispecific EGFR-cMet antibody comprises a heavy chain 1 (HC1) comprising the amino acid sequence of SEQ ID NO:17, HC2 comprising the amino acid sequence of SEQ ID NO:19, a light chain 1 (LC1) comprising the amino acid sequence of SEQ ID NO:18, and a LC2 comprising the amino acid sequence of SEQ ID NO:20.

18. The stable aqueous pharmaceutical composition of claim 2, comprising about 160 mg/mL of the bispecific EGFR-cMet antibody, about 30 mM acetate and/or pharmaceutically acceptable acetate salt, about 8.5% sucrose, about 1 mg/mL L-methionine with polysorbate 80 to a final concentration of about 0.06% (w/v), EDTA to a final concentration of about 20 μg/mL, and a human recombinant hyaluronidase enzyme PH20 (rHuPH20) to a final concentration of about 2,000 U/mL, wherein the stable aqueous pharmaceutical composition has pH about 5.7,
and wherein the bispecific EGFR-cMet antibody comprises a heavy chain 1 (HC1) comprising the amino acid sequence of SEQ ID NO:17, HC2 comprising the amino acid sequence of SEQ ID NO:19, a light chain 1 (LC1) comprising the amino acid sequence of SEQ ID NO:18, and a LC2 comprising the amino acid sequence of SEQ ID NO:20.

19. A method of treating cancer in a subject in need thereof, the method comprising administering to the subject the pharmaceutical composition of claim 1.

20. The method of claim 19, wherein the administration is subcutaneous.

21. The method of claim 19, wherein the cancer comprises lung cancer, squamous cell carcinoma of the head and neck (SCCHN), hepatocellular cancer (HCC), colorectal cancer (CRC), renal cell cancer (RCC), medullary thyroid cancer (MTC), gastroesophageal cancer (GEC), mesothelioma, breast cancer (BC) or ovarian cancer (OC).

22. The method of claim 21, wherein the cancer comprises non-small cell lung cancer (NSCLC).

23. The method of claim 21, wherein the cancer comprises colorectal cancer (CRC).

24. The method of claim 21, wherein the cancer comprises squamous cell carcinoma of the head and neck (SCCHN).

25. A method for preparing a stable aqueous pharmaceutical composition of a bispecific antibody targeting EGFR and cMet, the bispecific antibody targeting EGFR and cMet comprising a first heavy chain (HC1) comprising a HC1 variable region (VH1) comprising a heavy chain complementarity determining region 1 (HCDR1), a HCDR2, and a HCDR3 comprising the amino acid sequences of SEQ ID NOs: 1, 2, and 3, respectively; a first light chain (LC1) comprising a LC1 variable region (VL1) comprising a light chain complementarity determining region 1 (LCDR1), a LCDR2, and a LCDR3 comprising the amino acid sequences of SEQ ID NOs: 4, 5, and 6, respectively; a second heavy chain (HC2) comprising a HC2 variable region (VH2) comprising a HCDR1, a HCDR2, and a HCDR3 comprising the amino acid sequences of SEQ ID NOs: 7, 8, and 9, respectively; and a second light chain (LC2) comprising a LC2 variable region (VL2) comprising a LCDR1, a LCDR2, and a LCDR3 comprising the amino acid sequences of SEQ ID NOs: 10, 11, and 12, respectively; the method comprising:
combining a composition comprising about 160 mg/mL of the bispecific antibody, about 30 mM acetate and/or pharmaceutically acceptable acetate salt, about 8.5% sucrose, and about 1 mg/mL L-methionine with polysorbate 80 to a final concentration of about 0.06% (w/v) and EDTA to a final concentration of about 20 μg/mL, optionally rHuPH20 to a final concentration of about 2,000 U/mL, wherein the stable aqueous pharmaceutical composition has pH about 5.7.

26. The method of claim 25, wherein the bispecific EGFR-cMet antibody comprises an HC1 variable region comprising the amino acid sequence of SEQ ID NO:13; a LC1 variable region comprising the amino acid sequence of SEQ ID NO:14; a HC2 variable region comprising the amino acid sequence of SEQ ID NO:15; and a LC2 variable region comprising the amino acid sequence of SEQ ID NO:16.

27. The method of claim 25, wherein the antibody comprises a heavy chain 1 (HC1) comprising the amino acid sequence of SEQ ID NO:17; a light chain 1 (LC1) comprising the amino acid sequence of SEQ ID NO:18; a HC2 comprising the amino acid sequence of SEQ ID NO:19; and a LC2 comprising the amino acid sequence of SEQ ID NO:20.

28. The method of claim 25, wherein the antibody is amivantamab or a biosimilar thereof.

29. A kit comprising the stable aqueous pharmaceutical composition of claim 1 and instructions for use thereof.

30. An article of manufacture comprising a container holding a stable aqueous pharmaceutical composition in accordance with claim 1.

31. The article of manufacture according to claim 30, wherein the container is a vial with a stopper pierceable by a syringe.

32. The article of manufacture according to claim 31, wherein the vial is a single-use vial.

33. A method of reducing infusion-related reactions in a subject treated with amivantamab comprising subcutaneously administering the stable aqueous pharmaceutical formulation of claim 1 to the subject.
